(19) United States
(12) Patent Application Publication
(10) Pub. No.: US 2026/0184727 A1
(43) Pub. Date: Jul. 2, 2026

(54) SOLID FORMS OF MACROCYCLIC COMPOUNDS AND USES THEREOF

(71) Applicant: AMGEN INC., Thousand Oaks, CA (US)

(72) Inventors: Jayshil BHATT, Thousand Oaks, CA (US); Vilmali LOPEZ-MEJIAS, Thousand Oaks, CA (US); Prashant AGARWAL, Thousand Oaks, CA (US); Laszlo VISONTAI, Woodland Hills, CA (US); Yicong LIANG, Simi Valley, CA (US); Jake NEWMAN, Canoga Park, CA (US); Taylor Cassianna HARDIN, Sherman Oaks, CA (US); Adam Nicholas SMITH, Cambridge, MA (US); Rayan ALHASHIM, Thousand Oaks, CA (US); Xavier FARAJ, Thousand Oaks, CA (US)

(73) Assignee: AMGEN INC., Thousand Oaks, CA (US)

(21) Appl. No.: 19/288,056

(22) Filed: Aug. 1, 2025

Related U.S. Application Data
(63) Continuation of application No. PCT/US25/26585, filed on Apr. 28, 2025.
(60) Provisional application No. 63/640,189, filed on Apr. 29, 2024.
(60) Provisional application No. 63/646,460, filed on May 13, 2024.

(51) Int. Cl. C07D 519/00; A61K 31/553; C07C 55/08

(52) U.S. Cl. CPC C07D 519/00; A61K 31/553; C07C 55/08; C07B 2200/13

(57) ABSTRACT

The disclosure provides solid forms of Compound A, as described herein. The disclosure also provides pharmaceutical compositions comprising the disclosed solid forms and methods of using the disclosed solid forms and pharmaceutical compositions (e.g., to treat cancer).

### CROSS REFERENCE TO RELATED APPLICATIONS

This application claims the benefit of priority to U.S. Provisional Patent Application No. 63/640,189, filed Apr. 29, 2024 and U.S. Provisional Application No. 63/646,460, filed May 13, 2024.

### FIELD

The present disclosure provides solid forms of Compound A

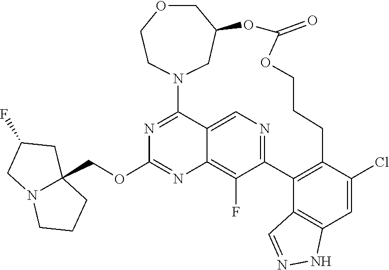 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

having activity as inhibitors of KRAS, for example, KRAS KRAS G12D, G12V, G12A, G12S, G12R, G13D, Q61H, Q61L, Q61R or G12C. This disclosure also provides pharmaceutical compositions comprising the solid forms, uses and methods of treating certain disorders such as cancer, including but not limited to hepatobiliary, ovarian, endometrial, pancreatic (e.g., pancreatic ductal adenocarcinoma (PDAC)), colorectal, or non-small cell lung cancer (e.g., lung adenocarcinoma (LUAD)).

### BACKGROUND

The Kirsten rat sarcoma viral oncogene homolog (KRAS) is a G-protein that couples extracellular mitogenic signaling to intracellular, pro-proliferative responses. KRAS serves as an intracellular “on/off” switch. Mitogen stimulation induces the binding of GTP to KRAS, bringing about a conformational change which enables the interaction of KRAS with downstream effector proteins, leading to cellular proliferation. Normally, pro-proliferative signaling is regulated by the action of GTPase-activating proteins (GAPs), which return KRAS to its GDP-bound, non-proliferative state. Mutations in KRAS impair the regulated cycling of KRAS between these GDP- and GTP-bound states, leading to the accumulation of the GTP-bound active state and dysregulated cellular proliferation (Simanshu et al., 2017).

Attempts to develop inhibitors of mutated KRAS proteins historically have been thwarted by the absence of druggable pockets on the surface of the protein (Cox et al., 2014). In 2013, Shokat and colleagues identified covalent inhibitors of a common (O'Bryan, 2019) oncogenic mutant of KRAS, KRAS G12C, which bound to a previously unrecognized allosteric pocket on GDP-KRAS G12C and prevented its subsequent activation (Ostrem et al., 2013). This discovery brought about significant new efforts in the KRAS inhibitor research, which have recently culminated in the entry of KRAS inhibitors in human clinical trials.

While some progress has been made on KRAS inhibitors, there is a continued interest and effort to develop inhibitors of KRAS, particularly inhibitors of other KRAS such as KRAS G12D, G12V, G12A, G12S, G12R, G13D, Q61H, Q61L, Q61R or G12C.

In view of the foregoing, there is a need for solid forms of naphthyridine compounds, including crystalline forms and amorphous forms, as well as solvates and co-crystals thereof and pharmaceutically acceptable salts thereof.

### SUMMARY

The disclosure provides solid forms of Compound A

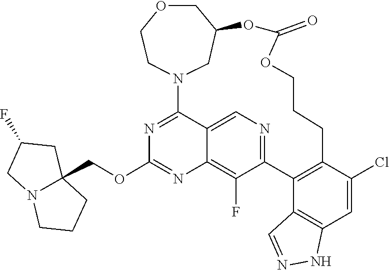 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

(Compound A), including crystalline forms, amorphous forms, solvates, and co-crystals, as well as pharmaceutically acceptable salts of the disclosed forms.

In some embodiments, the disclosure provides salts of Compound A, wherein the salts are pharmaceutically acceptable salts (e.g., maleate, citrate, glycine hydrochloride, malonate, oxalate, tosylate, saccharinate, hydrobromide, hydrochloride, edisylate, besylate, esylate, and hemiedisylate).

In some embodiments, the disclosure provides co-crystals of Compound A comprising Compound A and a coformer (e.g., quercetin, acetazolamide, L-tartartic acid, and glutaric acid).

In some embodiments, the disclosure provides crystalline forms of Compound A as a free base hydrate.

In some embodiments, the disclosure provides an amorphous form of Compound A as free base or salt form, such as an amorphous oxalate of Compound A (“Form A1”), as described herein, or an amorphous tosylate of Compound A (“Form A1-A”), as described herein, or an amorphous saccharinate of Compound A (“Form A1-B”), as described herein, or an amorphous citrate of Compound A (“Form A1-C”), as described herein, or an amorphous free base of Compound A (“Form A2”), as described herein, or an amorphous besylate of Compound A (“Form A1-D”), as described herein, or as an amorphous esylate of Compound A (“Form A1-E”), as described herein.

In some embodiments, the disclosure provides pharmaceutical compositions comprising the disclosed solid forms of Compound A, as well as methods using the disclosed compositions.

### BRIEF DESCRIPTION OF DRAWINGS

FIG. 1 depicts a form map of the various solid forms of Compound A disclosed herein.

FIG. 2 depicts the X-ray powder diffraction (XRPD) pattern of Compound A malonate salt anhydrous (“Form 1E”).

FIG. 3 depicts the differential scanning calorimetry (DSC) thermograph of Compound A malonate salt anhydrous (“Form 1E”).

FIG. 4 depicts the thermogravimetric analysis (TGA) thermograph of Compound A malonate salt anhydrous (“Form 1E”).

FIG. 5 depicts the ¹H NMR spectrum of Compound A malonate salt anhydrous (“Form 1E”).

FIG. 5A depicts the ¹³C ssNMR spectrum of Compound A malonate salt anhydrous (“Form 1E”).

FIG. 5B depicts a comparison of the ¹³C ssNMR of Form 1E and Compound A free base hydrate.

FIG. 5C depicts the plasma concentrations of Form 1E when administered in dogs.

FIG. 5D depicts the total plasma concentrations of Form 1E in dogs when administered to dogs fed a kibble diet.

FIG. 5E depicts the total plasma concentrations of Form 1E in dogs when administered to dogs fed a high fat diet.

FIG. 6 depicts the dynamic vapor sorption (DVS) graph of Compound A malonate salt anhydrous (“Form 1E”).

FIG. 7 depicts the X-ray powder diffraction (XRPD) pattern of Compound A maleate salt hydrate (“Form 1A”).

FIG. 8 depicts the differential scanning calorimetry (DSC) thermograph of Compound A maleate salt hydrate (“Form 1A”).

FIG. 9 depicts the thermogravimetric analysis (TGA) thermograph of Compound A maleate salt hydrate (“Form 1A”).

FIG. 10 depicts the ¹H NMR spectrum of Compound A maleate salt hydrate (“Form 1A”).

FIG. 11 depicts the dynamic vapor sorption (DVS) graph of Compound A maleate salt hydrate (“Form 1A”).

FIG. 12 depicts the X-ray powder diffraction (XRPD) pattern of Compound A citrate salt hydrate (“Form 1B”).

FIG. 13 depicts the differential scanning calorimetry (DSC) thermograph of Compound A citrate salt hydrate (“Form 1B”).

FIG. 14 depicts the thermogravimetric analysis (TGA) thermograph of Compound A citrate salt hydrate (“Form 1B”).

FIG. 15 depicts the ¹H NMR spectrum of Compound A citrate salt hydrate (“Form 1B”).

FIG. 16 depicts the dynamic vapor sorption (DVS) graph of Compound A citrate salt hydrate (“Form 1B”)

FIG. 17 depicts the X-ray powder diffraction (XRPD) pattern of Compound A hydrobromide (“Form S-L”).

FIG. 18 depicts the differential scanning calorimetry (DSC) thermograph of Compound A hydrobromide (“Form S-L”).

FIG. 19 depicts the ¹H NMR spectrum of Compound A hydrobromide (“Form S-L”).

FIG. 20 depicts the X-ray powder diffraction (XRPD) pattern of Compound A hydrochloride (“Form S-T”).

FIG. 21 depicts the differential scanning calorimetry (DSC) thermograph of Compound A hydrochloride (“Form S-T”).

FIG. 22 depicts the thermogravimetric analysis (TGA) thermograph of Compound A hydrochloride (“Form S-T”).

FIG. 23 depicts the ¹H NMR spectrum of Compound A hydrochloride (“Form S-T”).

FIG. 24 depicts the X-ray powder diffraction (XRPD) pattern of Compound A edisylate salt (“Form 1F”).

FIG. 25 depicts the differential scanning calorimetry (DSC) thermograph of Compound A edisylate salt (“Form 1F”).

FIG. 26 depicts the thermogravimetric analysis (TGA) thermograph of Compound A edisylate salt (“Form 1F”).

FIG. 27 depicts the X-ray powder diffraction (XRPD) pattern of Compound A hemiedisylate salt (“Form 1G”).

FIG. 28 depicts the differential scanning calorimetry (DSC) thermograph of Compound A hemiedisylate salt (“Form 1G”).

FIG. 29 depicts the thermogravimetric analysis (TGA) thermograph of Compound A hemiedisylate salt (“Form 1G”).

FIG. 30 depicts the ¹H NMR spectrum of Compound A hemiedisylate salt (“Form 1G”).

FIG. 31 depicts the X-ray powder diffraction (XRPD) pattern of Compound A quercetin co-crystal (“Form C-P”).

FIG. 32 depicts the differential scanning calorimetry (DSC) thermograph of Compound A quercetin co-crystal (“Form C-P”).

FIG. 33 depicts the thermogravimetric analysis (TGA) thermograph of Compound A quercetin co-crystal (“Form C-P”).

FIG. 34 depicts the dynamic vapor sorption (DVS) graph of Compound A quercetin co-crystal (“Form C-P”).

FIG. 35 depicts the X-ray powder diffraction (XRPD) pattern of Compound A glycine hydrochloride co-crystal hydrate (“Form C1”).

FIG. 36 depicts the differential scanning calorimetry (DSC) thermograph of Compound A glycine hydrochloride co-crystal hydrate (“Form C1”).

FIG. 37 depicts the thermogravimetric analysis (TGA) thermograph of Compound A glycine hydrochloride co-crystal hydrate (“Form C1”).

FIG. 38 depicts the ¹H NMR of Compound A glycine hydrochloride co-crystal hydrate (“Form C1”).

FIG. 39 depicts the dynamic vapor sorption (DVS) graph of Compound A glycine hydrochloride co-crystal hydrate (“Form C1”).

FIG. 40 depicts the X-ray powder diffraction (XRPD) pattern of Compound A acetazolamide co-crystal (“Form C-E”).

FIG. 41 depicts the X-ray powder diffraction (XRPD) pattern of Compound A L-tartaric acid co-crystal (“Form C-D”).

FIG. 42 depicts the X-ray powder diffraction (XRPD) pattern of Compound A glutaric acid co-crystal (“Form C-C”).

FIG. 43 depicts the X-ray powder diffraction (XRPD) pattern of Compound A free base hydrate (“Form 1”).

FIG. 44 depicts the differential scanning calorimetry (DSC) thermograph of Compound A free base hydrate (“Form 1”).

FIG. 45 depicts the thermogravimetric analysis (TGA) thermograph of Compound A free base hydrate (“Form 1”).

FIG. 46 depicts the ¹H NMR spectrum of Compound A free base hydrate (“Form 1”).

FIG. 47 depicts the X-ray powder diffraction (XRPD) pattern of Compound A free base hydrate (“Form 2”).

FIG. 48 depicts the thermogravimetric analysis (TGA) thermograph of Compound A free base hydrate (“Form 2”).

FIG. 49 depicts the differential scanning calorimetry (DSC) thermograph of amorphous Compound A oxalate (“Form A1”).

FIG. 50 depicts the thermogravimetric analysis (TGA) thermograph of amorphous Compound A oxalate (“Form A1”).

FIG. 51 depicts the ¹H NMR spectrum of amorphous Compound A oxalate (“Form A1”).

FIG. 52 depicts the X-ray powder diffraction (XRPD) pattern of amorphous Compound A oxalate (“Form A1”).

FIG. 53 depicts the differential scanning calorimetry (DSC) thermograph of amorphous Compound A tosylate (“Form A1-A”).

FIG. 54 depicts the thermogravimetric analysis (TGA) thermograph of amorphous Compound A tosylate (“Form A1-A”).

FIG. 55 depicts the ¹H NMR spectrum of amorphous Compound A tosylate (“Form A1-A”).

FIG. 56 depicts the X-ray powder diffraction (XRPD) pattern of amorphous Compound A tosylate (“Form A1-A”).

FIG. 57 depicts the differential scanning calorimetry (DSC) thermograph of amorphous Compound A saccharinate (“Form A1-B”).

FIG. 58 depicts the thermogravimetric analysis (TGA) thermograph of amorphous Compound A saccharinate (“Form A1-B”).

FIG. 59 depicts the ¹H NMR spectrum of amorphous Compound A saccharinate (“Form A1-B”).

FIG. 60 depicts the X-ray powder diffraction (XRPD) pattern of amorphous Compound A saccharinate (“Form A1-B”).

FIG. 61 depicts the differential scanning calorimetry (DSC) thermograph of amorphous Compound A citrate (“Form A1-C”).

FIG. 62 depicts the thermogravimetric analysis (TGA) thermograph of amorphous Compound A citrate (“Form A1-C”).

FIG. 63 depicts the ¹H NMR spectrum of amorphous Compound A citrate (“Form A1-C”).

FIG. 64 depicts the X-ray powder diffraction (XRPD) pattern of amorphous Compound A citrate (“Form A1-C”).

FIG. 65 depicts the differential scanning calorimetry (DSC) thermograph of amorphous Compound A free base (“Form A2”).

FIG. 66 depicts the thermogravimetric analysis (TGA) thermograph of amorphous Compound A free base (“Form A2”).

FIG. 67 depicts the X-ray powder diffraction (XRPD) pattern of amorphous Compound A free base (“Form A2”).

FIG. 68 depicts the differential scanning calorimetry (DSC) thermograph of amorphous Compound A besylate (“Form A1-D”).

FIG. 69 depicts the thermogravimetric analysis (TGA) thermograph of amorphous Compound A besylate (“Form A1-D”).

FIG. 70 depicts the ¹H NMR spectrum of amorphous Compound A besylate (“Form A1-D”).

FIG. 71 depicts the X-ray powder diffraction (XRPD) pattern of amorphous Compound A besylate (“Form A1-D”).

FIG. 72 depicts the differential scanning calorimetry (DSC) thermograph of amorphous Compound A esylate (“Form A1-E”).

FIG. 73 depicts the thermogravimetric analysis (TGA) thermograph of amorphous Compound A esylate (“Form A1-E”).

FIG. 74 depicts the ¹H NMR spectrum of amorphous Compound A esylate (“Form A1-E”).

FIG. 75 depicts the X-ray powder diffraction (XRPD) pattern of amorphous Compound A esylate (“Form A1-E”).

FIG. 76 depicts the ¹³C spectrum of Compound A free base hydrate Form 1.

FIG. 77 depicts the ¹⁹F spectrum of Compound A free base hydrate Form 1.

FIG. 78 depicts the ¹⁹F spectrum of Compound A malonate salt (“Form 1E”)

### DETAILED DESCRIPTION

Provided herein are solid forms of Compound A, which has the following chemical structure:

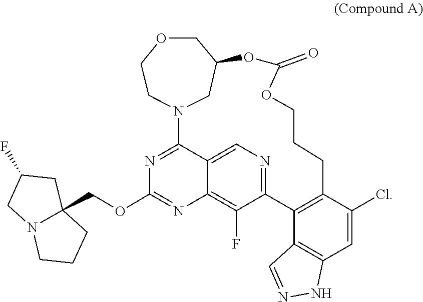 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

Compound A is an inhibitor of mutant KRAS proteins. The disclosed solid forms include crystalline forms and amorphous forms of Compound A free base, as well as crystalline salts, amorphous salts, co-crystals, and solvates of Compound A. The disclosed solid forms can have unique physical properties which are advantageous for new pharmaceutical compositions of Compound A.

Also disclosed herein are pharmaceutical compositions comprising the disclosed solid forms of Compound A and methods of treating cancer in a subject in need thereof, comprising administering to the subject a therapeutically effective amount of the disclosed solid forms of Compound A.

As used herein, the term “solid forms” refers to crystalline forms, amorphous forms, salts, co-crystals, or solvates, including but not limited to the specific solid forms disclosed herein. In some instances, the term “crystalline form” is used herein to refer to the various crystalline forms disclosed herein (e.g., free base forms, salts, solvates, and/or co-crystals).

As used herein, the term “salt” refers to a zwitterionic compound consisting of a cation and an anion. As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al. describe pharmaceutically acceptable salts in detail in J. Pharmaceutical Sciences, 1977, 66, 1-19, which is incorporated herein by reference. Pharmaceutically acceptable salts of the compounds of the disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, trifluoroacetic acid, oxalic acid, maleic acid, tartaric acid, citric acid, succinic acid or malonic acid or by using other methods used in the art such as ion exchange. Other illustrative pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, glutamate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts of compounds containing a carboxylic acid or other acidic functional group can be prepared by reacting with a suitable base. Such salts include, but are not limited to, alkali metal, alkaline earth metal, aluminum salts, ammonium, N+(C1-4alkyl) 4 salts, and salts of organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine. This invention also envisions the quaternization of any basic nitrogen-containing groups of the compounds disclosed herein. Water or oil-soluble or dispersible products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

In some embodiments, the disclosure provides pharmaceutically acceptable salts and/or co-crystals selected from maleate, citrate, glycine hydrochloride, malonate, oxalate, tosylate, saccharinate, hydrobromide, hydrochloride, edisylate, quercetin, acetazolamide, besylate, esylate, hemiedisylate, L-tartaric acid, and glutaric acid.

As used herein, the term “co-crystal” refers to a crystalline material comprising two or more compounds at ambient temperature (e.g., 20-25° C.), of which at least two are held together by weak interaction, wherein at least one of the compounds is a co-crystal former and the other is Compound A. Weak interaction is being defined as an interaction which is neither ionic nor covalent and includes for example: hydrogen bonds, van der Waals forces, and pi-pi interactions. The term “co-crystal” includes solvate forms.

As used herein, the term “amorphous form” or “amorphous” means a material that lacks long range order and as such does not show distinct X-ray diffraction peaks, i.e., a Bragg diffraction peak. The XRPD pattern of an amorphous material is characterized by one or more amorphous halos. As used herein, the term “amorphous halo” refers to an approximately bell-shaped maximum in the X-ray powder pattern of an amorphous substance.

The term “therapeutically effective amount” as used herein refers to that amount of a compound disclosed herein that elicits a desired biological or medical response in a cell, a tissue, a system, or a subject.

The term “subject” refers to humans and other mammals. The term “mammal” as used herein includes, for example, humans, non-human primates, cattle, sheep, goats, pigs, horses, cats, dog, rabbits, rodents (e.g., rats or mice), and monkeys. Human subjects include neonates, infants, juveniles, adults, and geriatric subjects.

As used herein to refer to DSC data, “substantially” refers to a variance of ±3° C.

The disclosed solid forms of Compound A were prepared and analyzed as described in the Examples using one or more of the following techniques: X-ray powder diffraction (XRPD), differential scanning calorimetry (DSC), thermogravimetric analysis (TGA), dynamic vapor sorption (DVS), nuclear magnetic resonance spectroscopy (NMR) (e.g., ¹H NMR and ¹³C ssNMR).

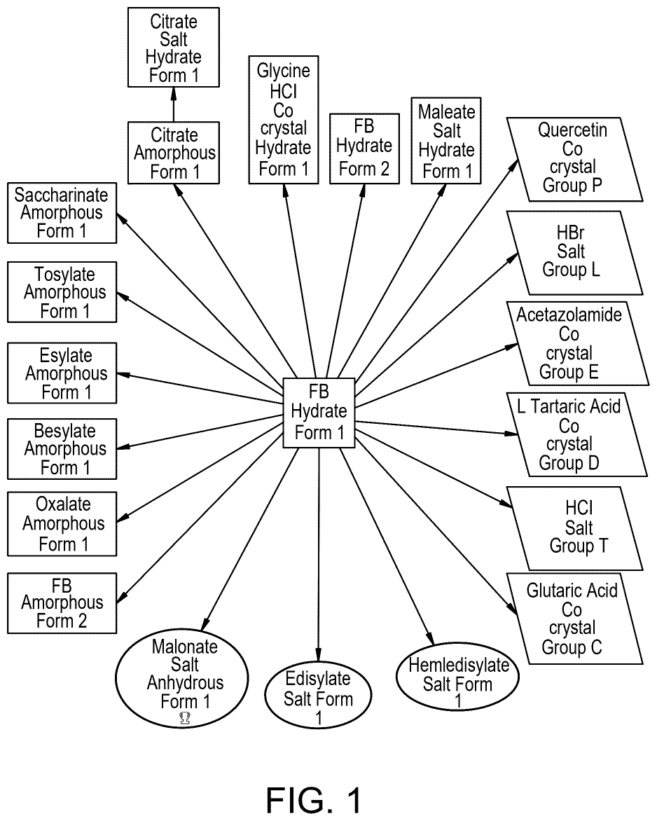

An overview of the various solid forms disclosed herein is shown in FIG. 1.

### Salts of Compound A

In some embodiments, the disclosure provides salts of Compound A, wherein the salts are pharmaceutically acceptable salts, as described herein. In some embodiments, the pharmaceutically acceptable salt comprises or is selected from maleate, citrate, malonate, oxalate, tosylate, saccharinate, hydrobromide, hydrochloride, edisylate, besylate, esylate, and hemiedisylate. In some embodiments, the pharmaceutically acceptable salt of Compound A comprises malonate. In some embodiments, the pharmaceutically acceptable salt of Compound A comprises maleate.

The disclosed salts of Compound A are of a suitable form. In some embodiments, the salts are crystalline, a solvate, or a combination thereof. By way of example, in some embodiments, the disclosed salts of Compound A are malonate anhydrous or maleate hydrate. In some embodiments, the salt of Compound A is a malonate anhydrous salt. In some embodiments, the salt of Compound A is maleate salt hydrate. In some embodiments, the salt of Compound A is a citrate hydrate. In some embodiments, the salt of Compound A is a hydrobromide. In some embodiments, the salt of Compound A is a hydrochloride. In some embodiments, the salt of Compound A is an edisylate salt. In some embodiments, the salt of Compound A is a hemiedisylate salt.

### Compound A Malonate Salt (“Form 1E”)

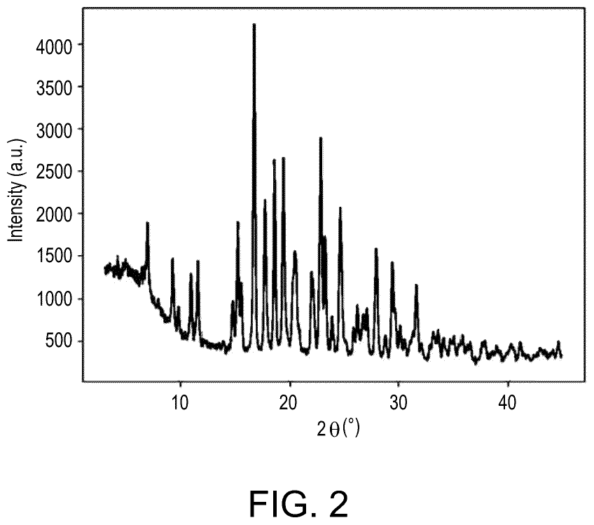

In some embodiments, the disclosure provides a malonate salt of Compound A. In some embodiments, the disclosure provides an anhydrous malonate salt of Compound A. In some embodiments, the disclosure provides a malonate salt of Compound A (“Form 1E”), characterized by an X-ray powder diffraction (XRPD) pattern comprising peaks at 16.7, 22.9, and 18.6±0.2° 2θ using CuKα radiation. In some embodiments, Form 1E is further characterized by a peak at 19.4±0.2° 2θ using CuKα radiation. In some embodiments, Form 1E is also further characterized by a peak at 17.7±0.2° 2θ using CuKα radiation. In some embodiments, Form 1E is also further characterized by peaks at 24.6, 15.2, 23.3, and 28.0±0.2° 2θ using CuKα radiation. In some embodiments, the disclosure provides Form 1E characterized by an X-ray powder diffraction (XRPD) pattern substantially as shown in FIG. 2.

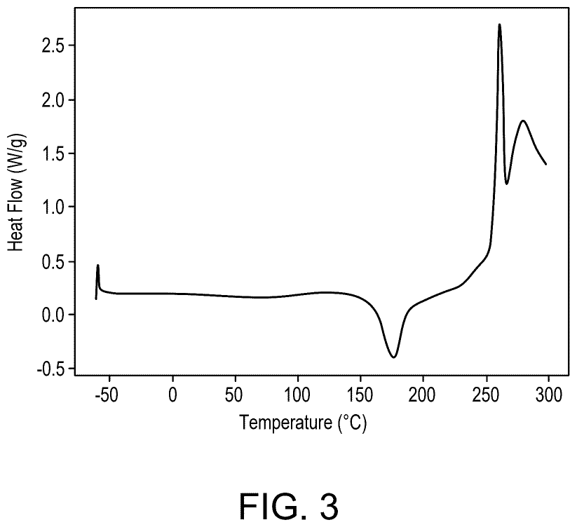

In some embodiments, the disclosure provides Form 1E characterized by a differential scanning calorimetry (DSC) thermograph substantially as shown in FIG. 3.

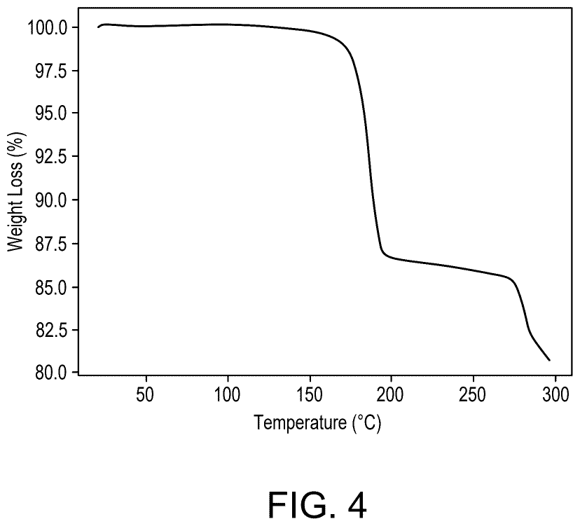

In some embodiments, the disclosure provides Form 1E characterized by a thermogravimetric analysis (TGA) thermograph substantially as shown in FIG. 4.

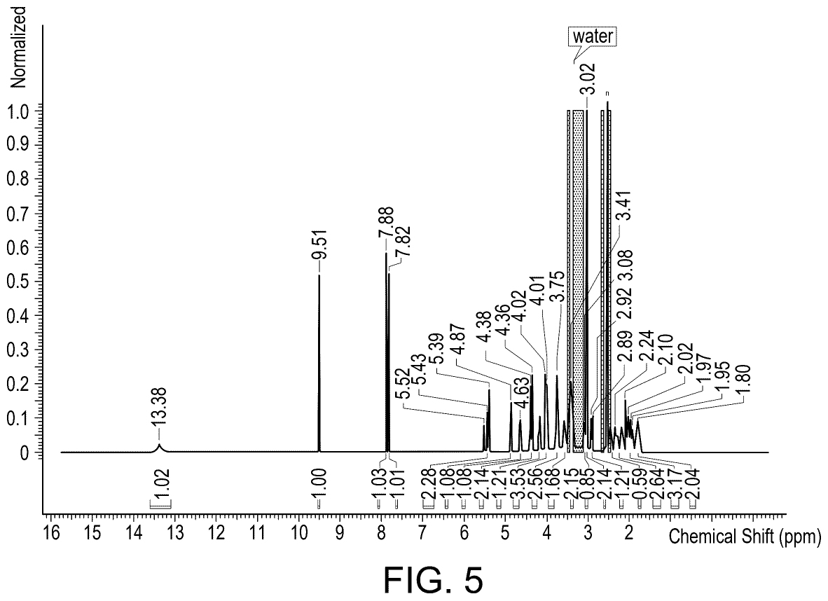

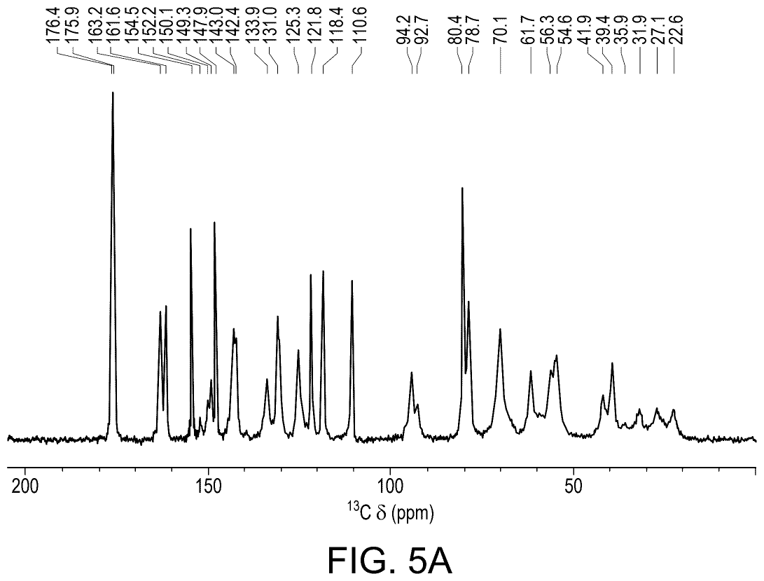

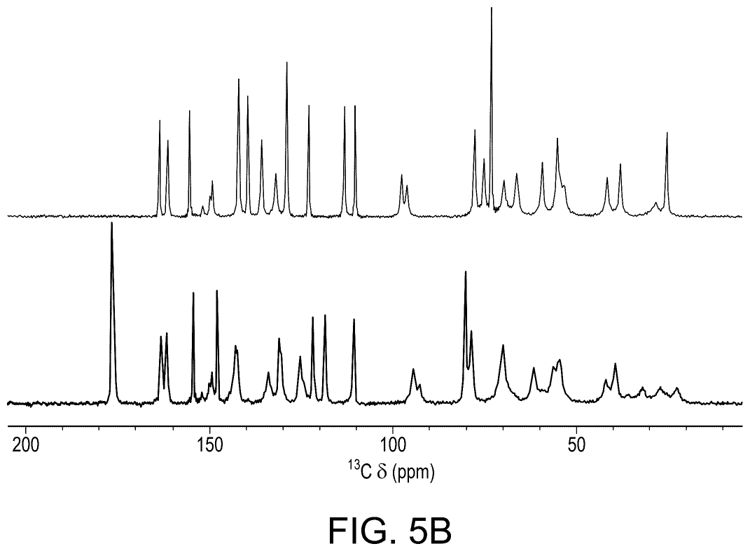

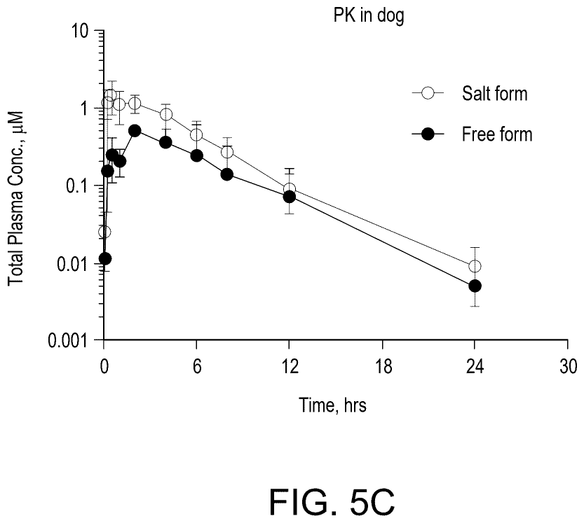

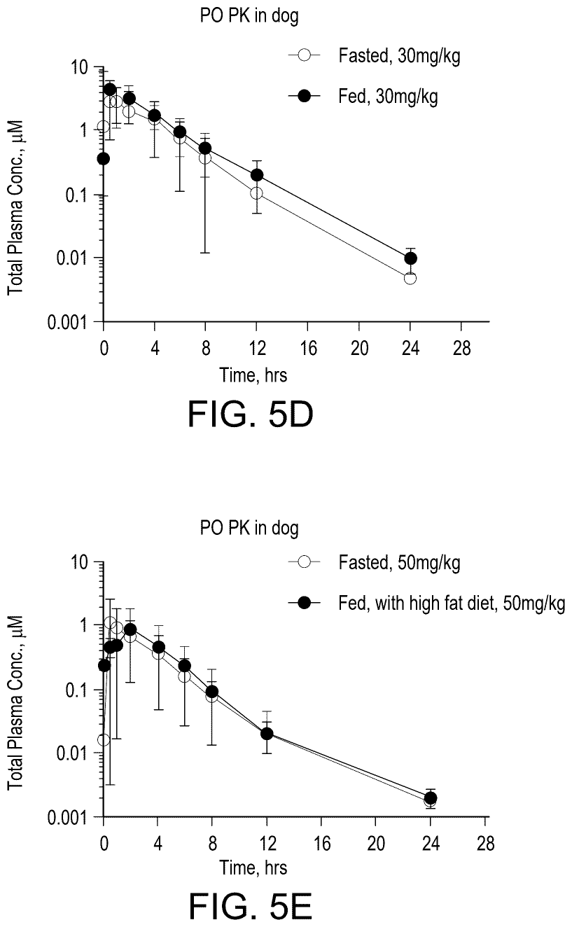

In some embodiments, the disclosure provides Form 1E characterized by a ¹H NMR spectrum substantially as shown in FIG. 5.

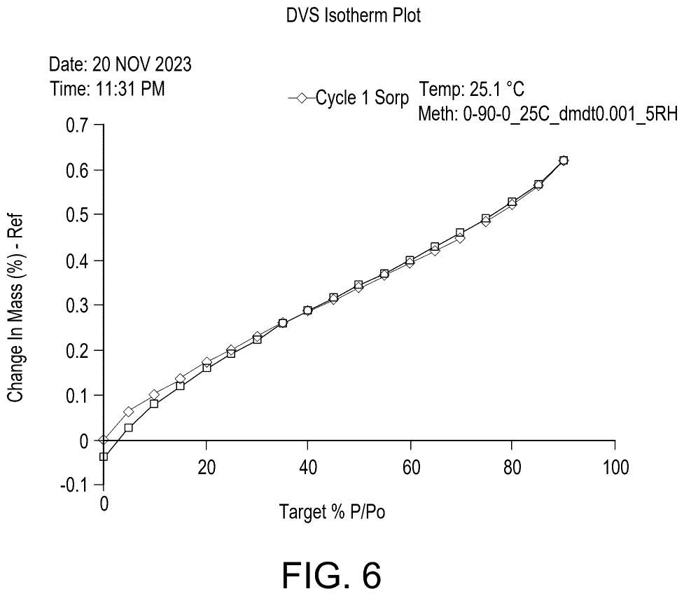

In some embodiments, the disclosure provides Form 1E characterized by a dynamic vapor sorption (DVS) graph substantially as shown in FIG. 6.

In some embodiments, the disclosure provides Form 1E, characterized by a ¹³C ssNMR spectrum comprising peaks 92.7, 176.4, and 80.4±0.2 ppm. In some embodiments, Form 1E is further characterized by a peak at 175.9±0.2 ppm. In some embodiments, Form 1E is further characterized by a peak at 147.9±0.2 ppm. In some embodiments, Form 1E is further characterized by peaks at 154.5, 118.4, and 121.8±0.2 ppm. In some embodiments, Form 1E is further characterized by peaks at 110.6, 78.7, 161.6, 163.2, and 131.0±0.2 ppm. In some embodiments, the disclosure provides Form 1E characterized by a ¹³C ssNMR spectrum substantially as shown in FIG. 5A.

### Compound A Maleate Salt (“Form 1A”)

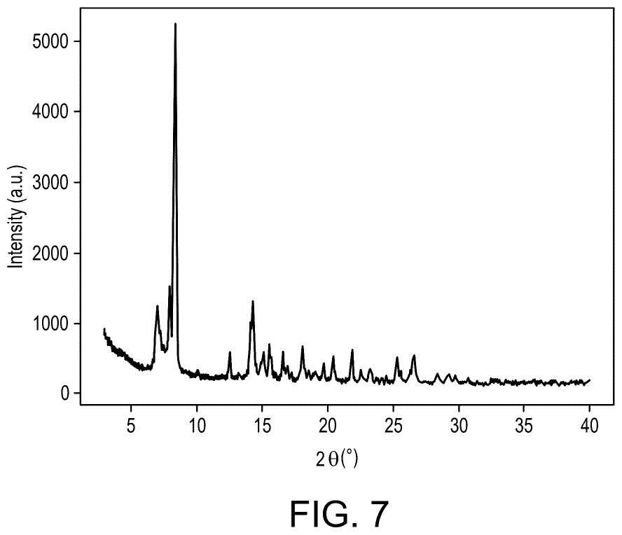

In some embodiments, the disclosure provides a maleate salt of Compound A. In some embodiments, the disclosure provides a maleate salt hydrate of Compound A. In some embodiments, the disclosure provides a maleate salt of Compound A (“Form 1A”), characterized by an X-ray powder diffraction (XRPD) pattern comprising peaks at 8.5, 22.0, and 8.1±0.2° 2θ using CuKα radiation. In some embodiments, Form 1A is further characterized by a peak at 14.5±0.2° 2θ using CuKα radiation. In some embodiments, Form 1A is also further characterized by a peak at 15.7±0.2° 2θ using CuKα radiation. In some embodiments, Form 1A is also further characterized by peaks at 26.7, 20.5, 25.4, and 16.7±0.2° 2θ using CuKα radiation. In some embodiments, the disclosure provides Form 1A characterized by an X-ray powder diffraction (XRPD) pattern substantially as shown in FIG. 7.

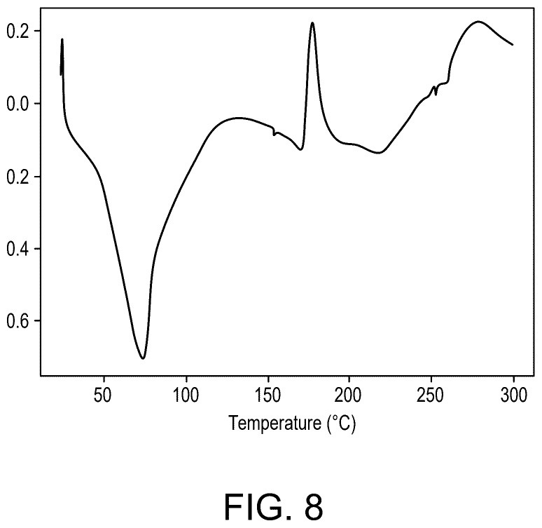

In some embodiments, the disclosure provides Form 1A characterized by a differential scanning calorimetry (DSC) thermograph substantially as shown in FIG. 8.

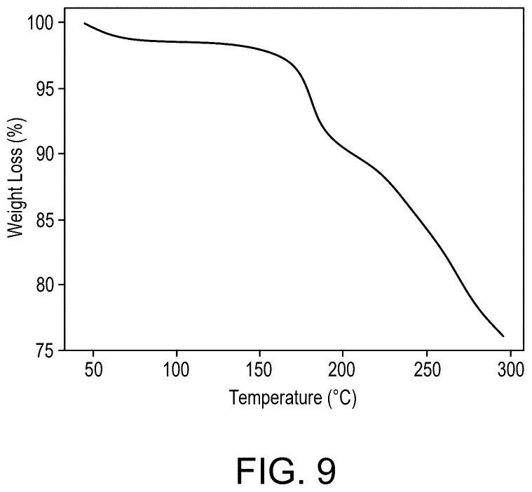

In some embodiments, the disclosure provides Form 1A characterized by a thermogravimetric analysis (TGA) thermograph substantially as shown in FIG. 9.

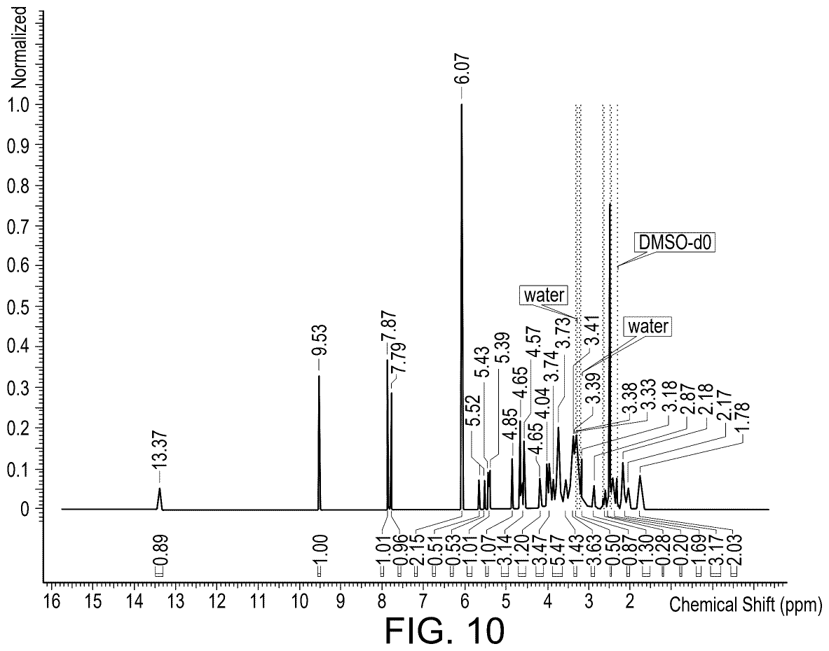

In some embodiments, the disclosure provides Form 1A characterized by a ¹H NMR spectrum substantially as shown in FIG. 10.

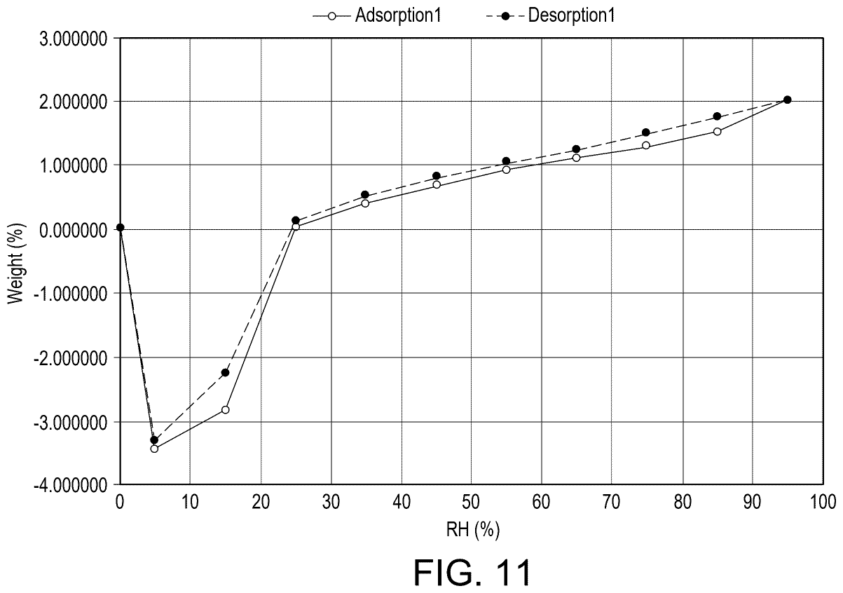

In some embodiments, the disclosure provides Form 1A characterized by a dynamic vapor sorption (DVS) graph substantially as shown in FIG. 11.

### Compound A Citrate Salt (“Form 1B”)

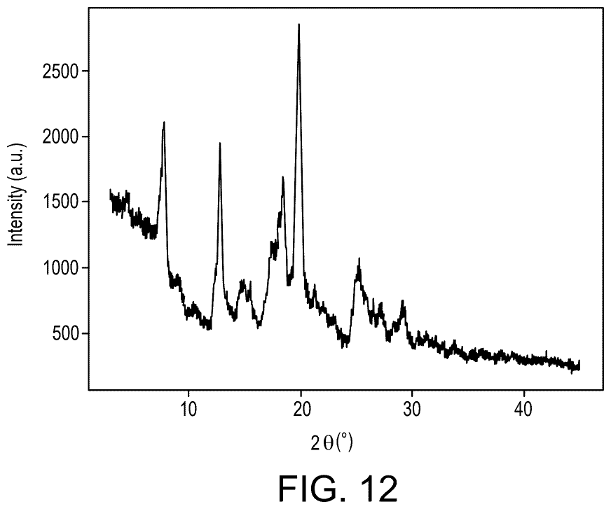

In some embodiments, the disclosure provides a citrate salt. In some embodiments, the disclosure provides a citrate salt hydrate. In some embodiments, the disclosure provides Compound A citrate salt (“Form 1B”), characterized by an X-ray powder diffraction (XRPD) pattern comprising peaks at 19.9, 12.8, and 18.5±0.2° 2θ using CuKα radiation. In some embodiments, Form 1B is further characterized by a peak at 7.8±0.2° 2θ using CuKα radiation. In some embodiments, Form 1B is also further characterized by a peak at 17.4±0.2° 2θ using CuKα radiation. In some embodiments, the disclosure provides Form 1B characterized by an X-ray powder diffraction (XRPD) pattern substantially as shown in FIG. 12.

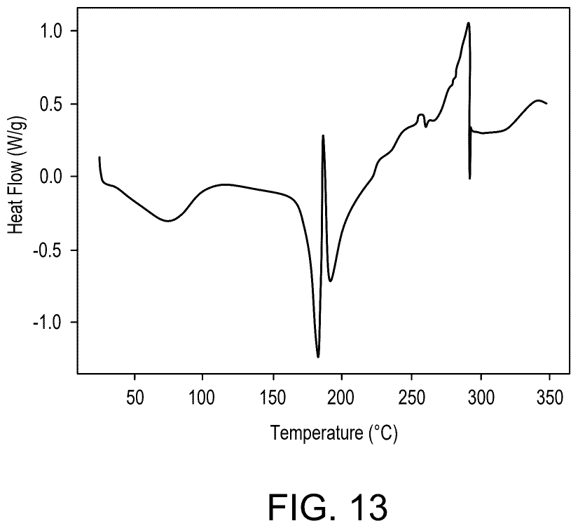

In some embodiments, the disclosure provides Form 1B characterized by a differential scanning calorimetry (DSC) thermograph substantially as shown in FIG. 13.

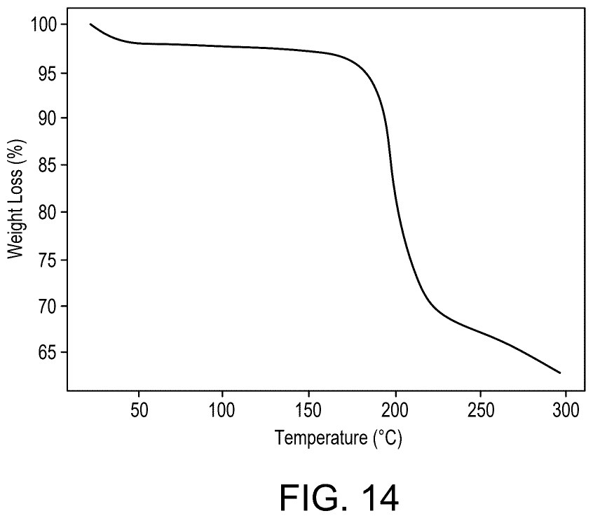

In some embodiments, the disclosure provides Form 1B characterized by a thermogravimetric analysis (TGA) thermograph substantially as shown in FIG. 14.

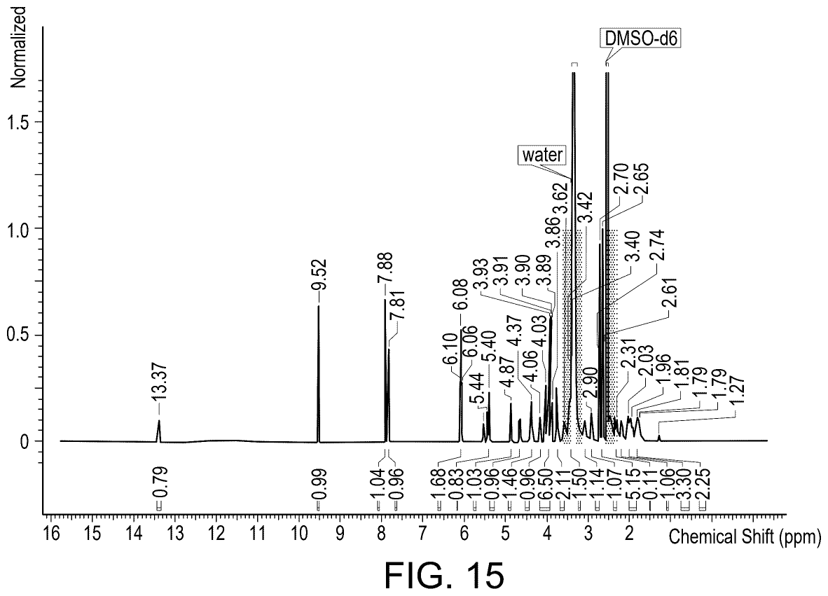

In some embodiments, the disclosure provides Form 1B characterized by a ¹H NMR spectrum substantially as shown in FIG. 15.

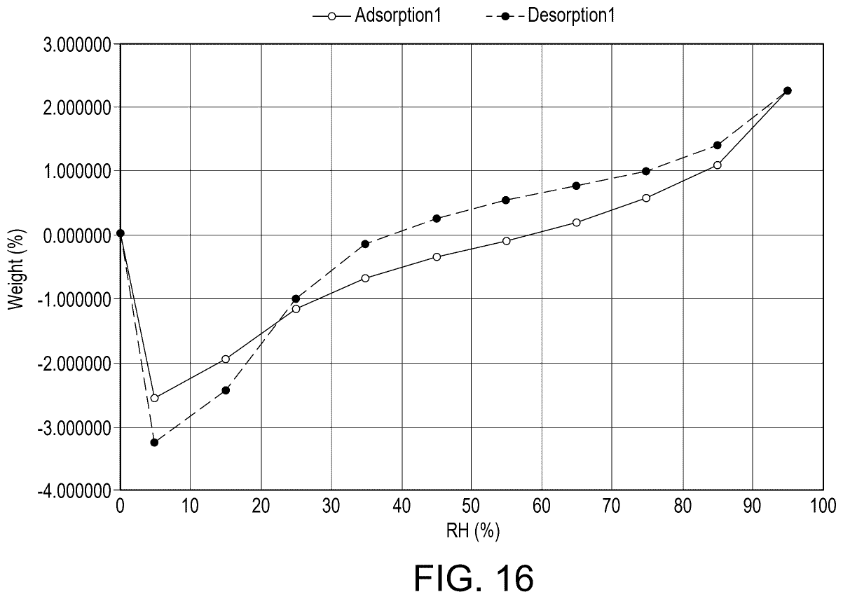

In some embodiments, the disclosure provides Form 1B characterized by a dynamic vapor sorption (DVS) graph substantially as shown in FIG. 16.

### Compound A Hydrobromide (“Form S-L”)

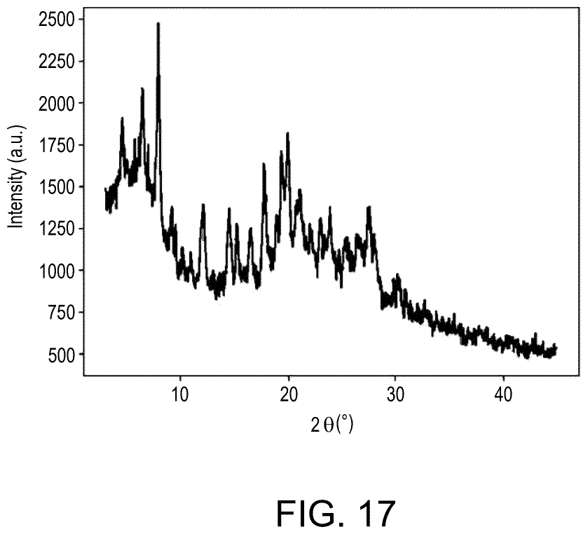

In some embodiments, the disclosure provides Compound A hydrobromide (“Form S-L”), characterized by an X-ray powder diffraction (XRPD) pattern comprising peaks at 7.0, 8.0, and 4.9±0.2° 2θ using CuKα radiation. In some embodiments, Form S-L is further characterized by a peak at 6.5±0.2° 2θ using CuKα radiation. In some embodiments, Form S-L is also further characterized by a peak at 17.9±0.2° 2θ using CuKα radiation. In some embodiments, the disclosure provides Form S-L characterized by an X-ray powder diffraction (XRPD) pattern substantially as shown in FIG. 17.

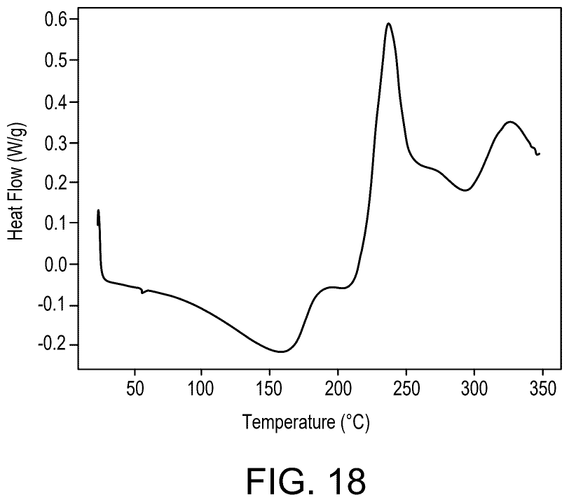

In some embodiments, the disclosure provides Form S-L characterized by a differential scanning calorimetry (DSC) thermograph substantially as shown in FIG. 18.

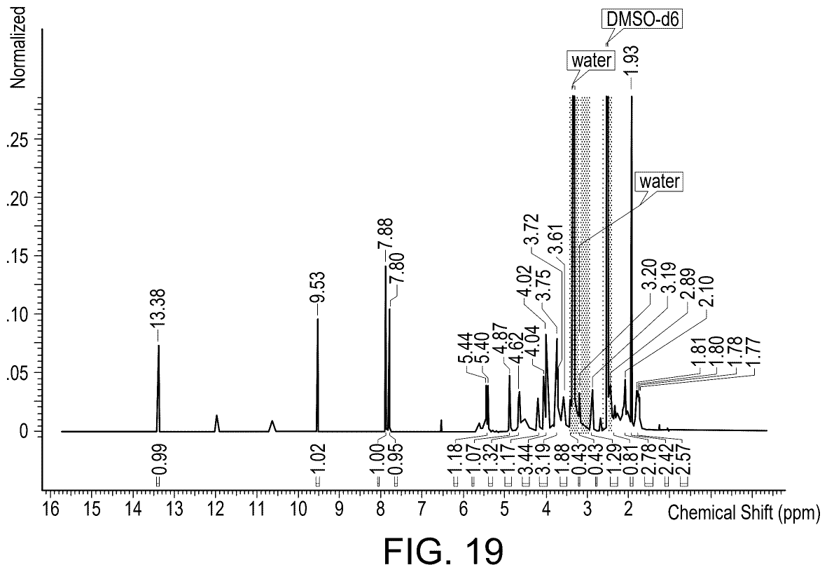

In some embodiments, the disclosure provides Form S-L characterized by a ¹H NMR spectrum substantially as shown in FIG. 19.

### Compound A Hydrochloride (“Form S-T”)

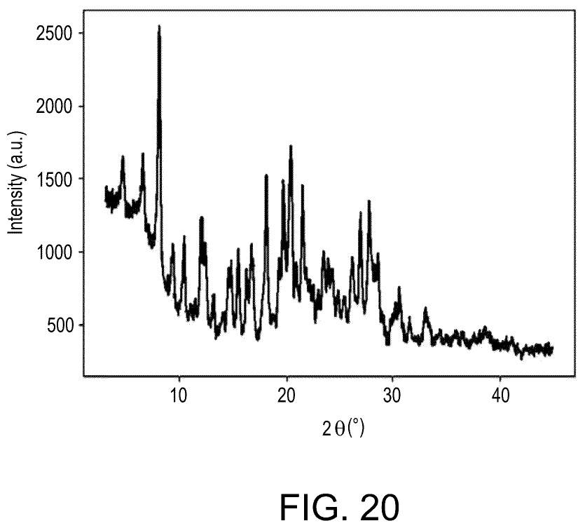

In some embodiments, the disclosure provides Compound A hydrochloride (“Form S-T”), characterized by an X-ray powder diffraction (XRPD) pattern comprising peaks at 8.1, 20.5, and 6.6±0.2° 2θ using CuKα radiation. In some embodiments, Form S-T is further characterized by a peak at 19.7±0.2° 2θ using CuKα radiation. In some embodiments, Form S-T is also further characterized by a peak at 21.5±0.2° 2θ using CuKα radiation. In some embodiments, Form S-T is also further characterized by peaks at 18.1, 12.1, and 27.8±0.2° 2θ using CuKα radiation. In some embodiments, the disclosure provides Form S-T characterized by an X-ray powder diffraction (XRPD) pattern substantially as shown in FIG. 20.

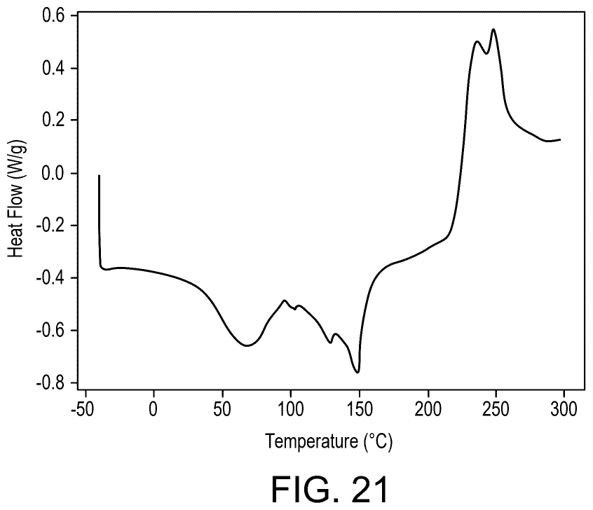

In some embodiments, the disclosure provides Form S-T characterized by a differential scanning calorimetry (DSC) thermograph substantially as shown in FIG. 21.

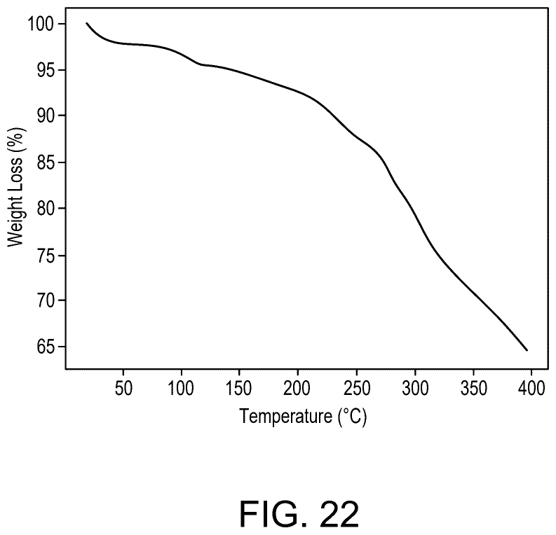

In some embodiments, the disclosure provides Form S-T characterized by a thermogravimetric analysis (TGA) thermograph substantially as shown in FIG. 22.

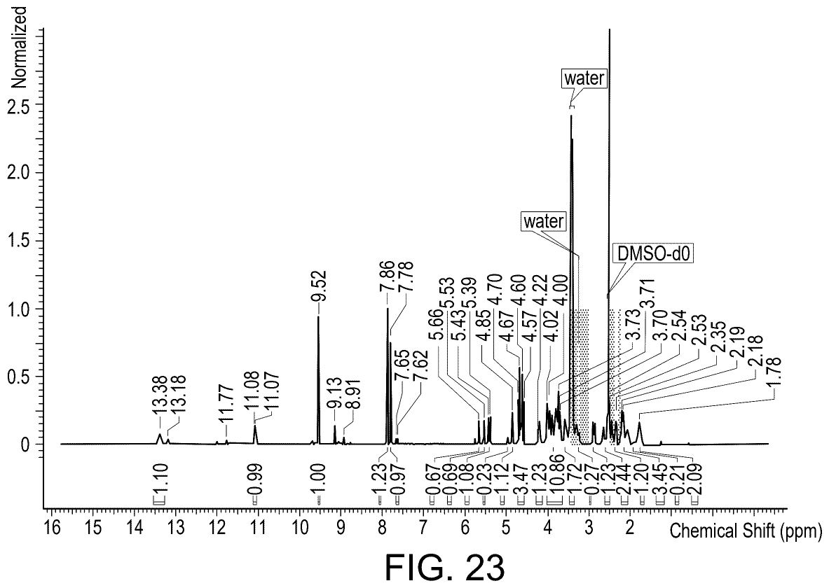

In some embodiments, the disclosure provides Form S-T characterized by a ¹H NMR spectrum substantially as shown in FIG. 23.

### Compound A Edisylate Salt (“Form 1F”)

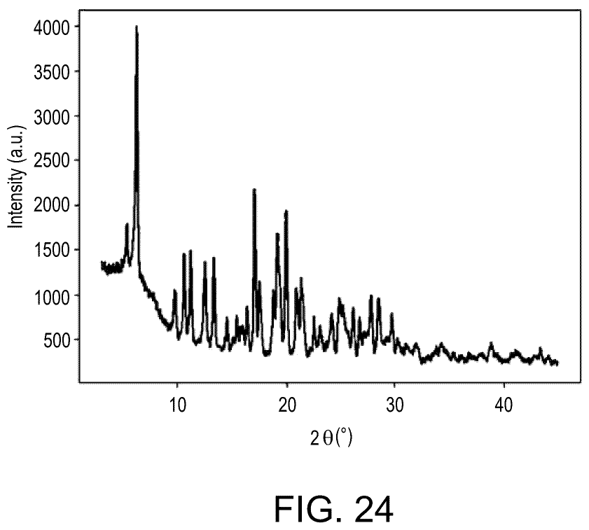

In some embodiments, the disclosure provides Compound A edisylate salt (“Form 1F”), characterized by an X-ray powder diffraction (XRPD) pattern comprising peaks at 6.3, 17.1, and 20.0±0.2° 2θ using CuKα radiation. In some embodiments, Form 1F is further characterized by a peak at 19.2±0.2° 2θ using CuKα radiation. In some embodiments, Form 1F is also further characterized by a peak at 13.3±0.2° 2θ using CuKα radiation. In some embodiments, Form 1F is also further characterized by peaks at 11.3, 10.7, and 12.5±0.2° 2θ using CuKα radiation. In some embodiments, the disclosure provides Form 1F characterized by an X-ray powder diffraction (XRPD) pattern substantially as shown in FIG. 24.

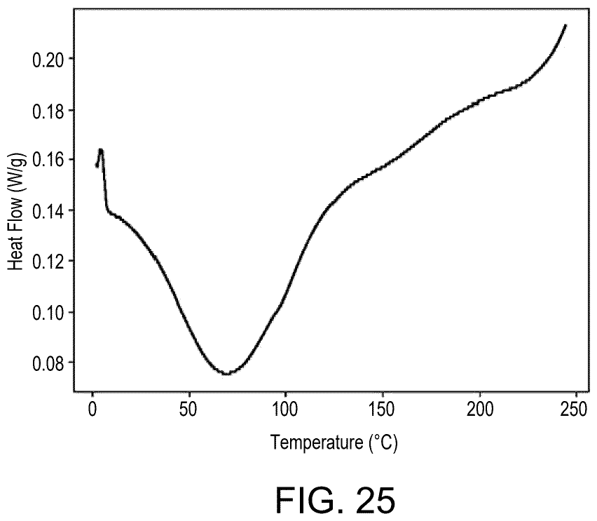

In some embodiments, the disclosure provides Form 1F characterized by a differential scanning calorimetry (DSC) thermograph substantially as shown in FIG. 25.

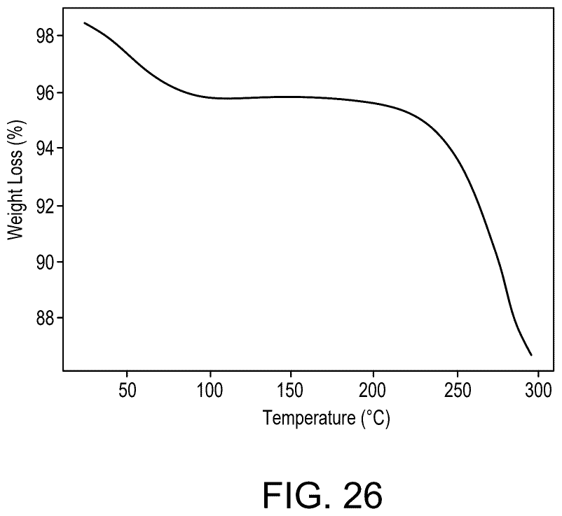

In some embodiments, the disclosure provides Form 1F characterized by a thermogravimetric analysis (TGA) thermograph substantially as shown in FIG. 26.

### Compound A Hemiedisylate Salt (“Form 1G”)

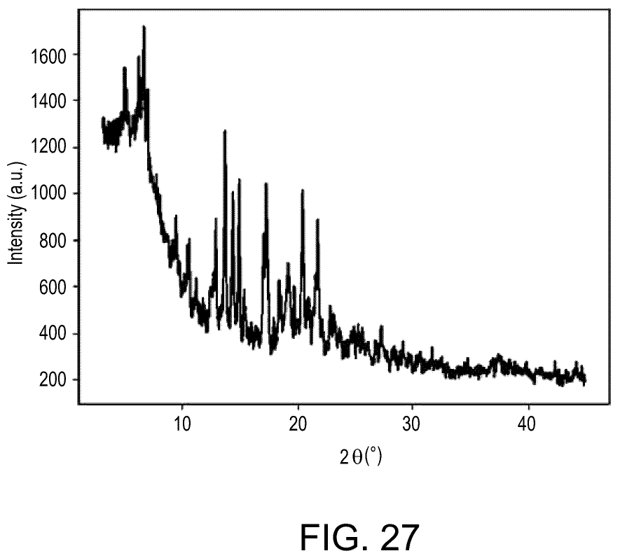

In some embodiments, the disclosure provides Compound A hemiedisylate salt (“Form 1G”), characterized by an X-ray powder diffraction (XRPD) pattern comprising peaks at 13.7, 6.7, and 20.5±0.2° 2θ using CuKα radiation. In some embodiments, Form 1G is further characterized by a peak at 17.3±0.2° 2θ using CuKα radiation. In some embodiments, Form 1G is also further characterized by a peak at 14.9±0.2° 2θ using CuKα radiation. In some embodiments, Form 1G is also further characterized by peaks at 14.3, 6.2, 21.8, 7.0, 5.0, 17.0, 12.9, 19.2, and 18.4±0.2° 2θ using CuKα radiation. In some embodiments, the disclosure provides Form 1G characterized by an X-ray powder diffraction (XRPD) pattern substantially as shown in FIG. 27.

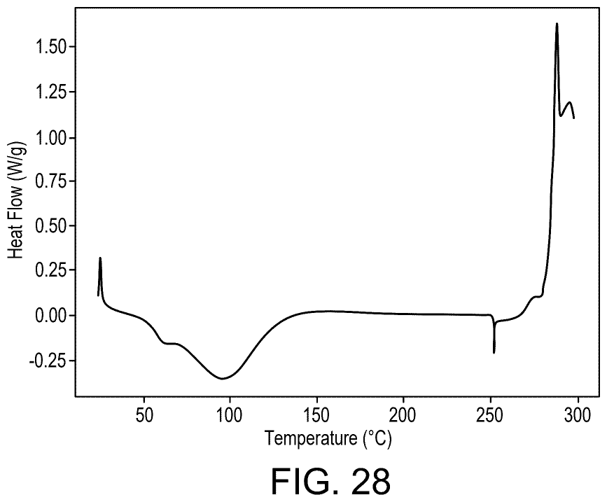

In some embodiments, the disclosure provides Form 1G characterized by a differential scanning calorimetry (DSC) thermograph substantially as shown in FIG. 28.

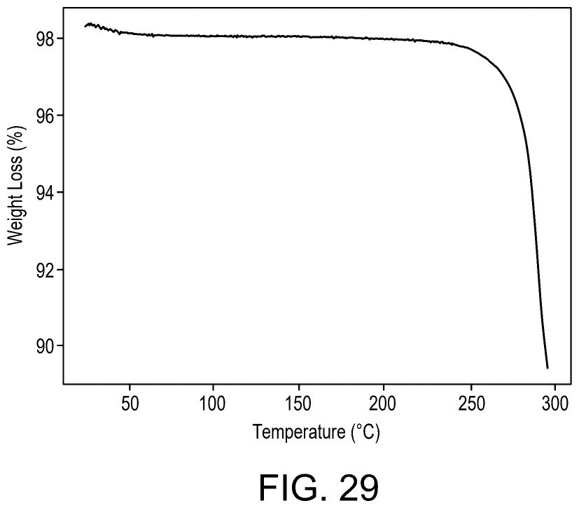

In some embodiments, the disclosure provides Form 1G characterized by a thermogravimetric analysis (TGA) thermograph substantially as shown in FIG. 29.

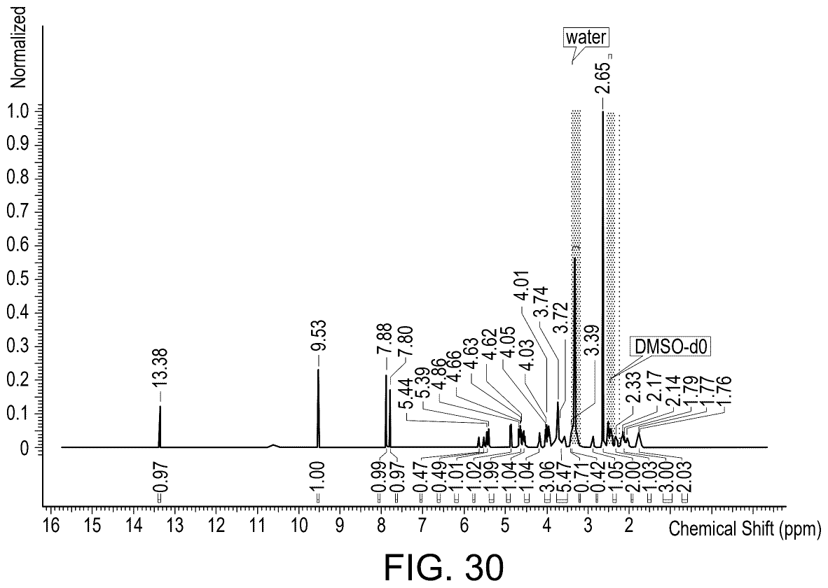

In some embodiments, the disclosure provides Form 1G characterized by a ¹H NMR spectrum substantially as shown in FIG. 30.

### Co-Crystal Forms of Compound A

In some embodiments, the disclosure provides co-crystals of Compound A with a coformer. As used herein, “coformer” refers to a molecule or compound that interacts with Compound A to form the co-crystal through noncovalent interactions (e.g., H-bonding, pi-pi stacking, or van der Waals forces). In some embodiments, the coformer comprises quercetin, acetazolamide, L-tartaric acid, glycine or a salt thereof (e.g., glycine hydrochloride), or glutaric acid or salt thereof. In some embodiments, the coformer comprises quercetin. In some embodiments, the coformer is quercetin. In some embodiments, the coformer comprises acetazolamide. In some embodiments, the coformer comprises L-tartaric acid. In some embodiments, the coformer comprises glycine hydrochloride. In some embodiments, the coformer comprises glutaric acid.

The disclosed co-crystals of Compound A are of a suitable form. In some embodiments, the coformer may be a solvate (e.g., hydrate) or a salt (hydrochloride salt).

### Compound A Quercetin Co-Crystal (“Form C-P”)

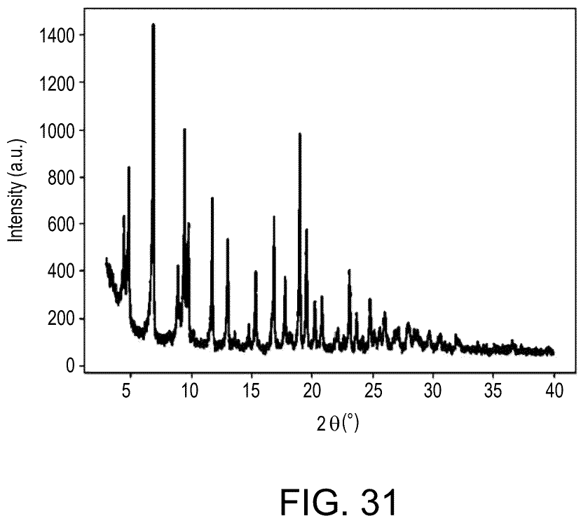

In some embodiments, the disclosure provides Compound A quercetin co-crystal (“Form C-P”), characterized by an X-ray powder diffraction (XRPD) pattern comprising peaks at 7.0, 19.1, and 9.5±0.2° 2θ using CuKα radiation. In some embodiments, Form C-P is further characterized by a peak at 4.9±0.2° 2θ using CuKα radiation. In some embodiments, Form C-P is also further characterized by a peak at 11.8±0.2° 2θ using CuKα radiation. In some embodiments, Form C-P is also further characterized by peaks at 13.1, 16.9, 9.8, 19.6±0.2° 2θ using CuKα radiation. In some embodiments, the disclosure provides Form C-P characterized by an X-ray powder diffraction (XRPD) pattern substantially as shown in FIG. 31.

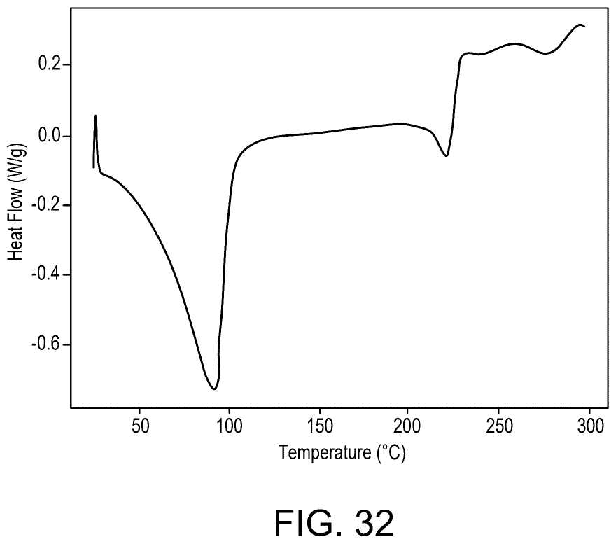

In some embodiments, the disclosure provides Form C-P characterized by a differential scanning calorimetry (DSC) thermograph substantially as shown in FIG. 32.

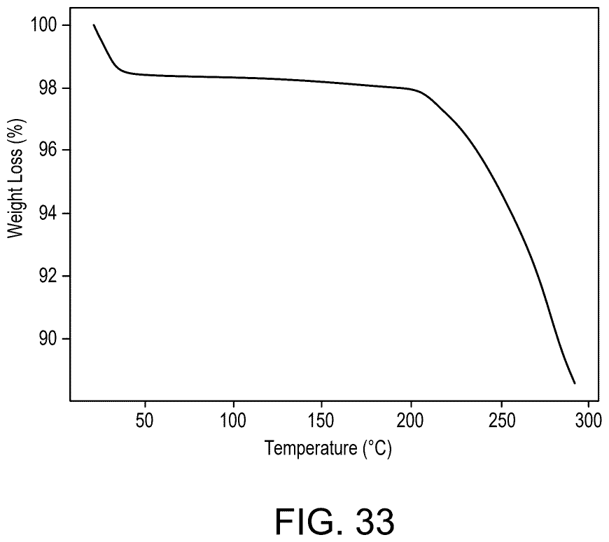

In some embodiments, the disclosure provides Form C-P characterized by a thermogravimetric analysis (TGA) thermograph substantially as shown in FIG. 33.

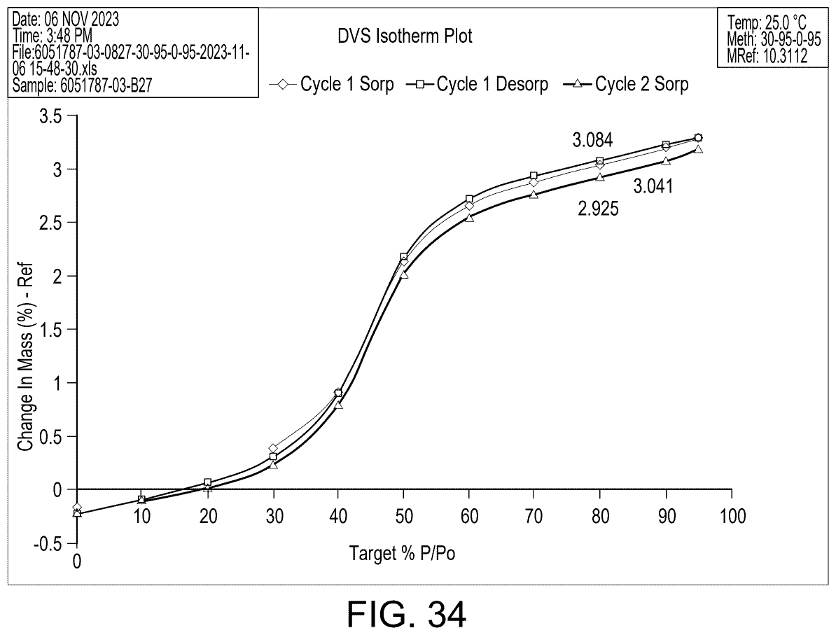

In some embodiments, the disclosure provides Form C-P characterized by a dynamic vapor sorption (DVS) graph substantially as shown in FIG. 34.

### Compound A Glycine Hydrochloride Co-Crystal Hydrate (“Form C1”)

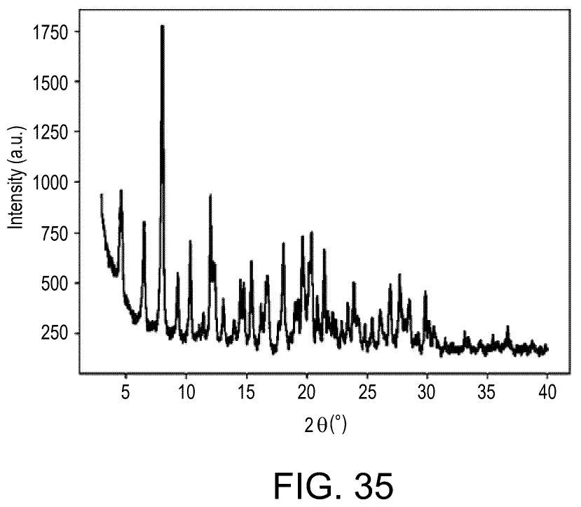

In some embodiments, the disclosure provides Compound A glycine hydrochloride co-crystal hydrate (“Form C1”), characterized by an X-ray powder diffraction (XRPD) pattern comprising peaks at 8.1, 18.0, and 23.8±0.2° 2θ using CuKα radiation. In some embodiments, Form C1 is further characterized by a peak at 12.0±0.2° 2θ using CuKα radiation. In some embodiments, Form C1 is also further characterized by a peak at 20.5±0.2° 2θ using CuKα radiation. In some embodiments, Form C1 is also further characterized by peaks at 28.5, 19.7, and 6.6±0.2° 2θ using CuKα radiation. In some embodiments, the disclosure provides Form C1 characterized by an X-ray powder diffraction (XRPD) pattern substantially as shown in FIG. 35.

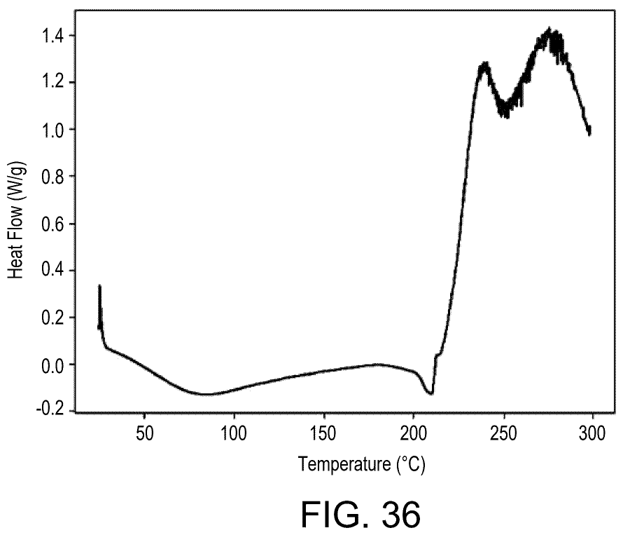

In some embodiments, the disclosure provides Form C1 characterized by a differential scanning calorimetry (DSC) thermograph substantially as shown in FIG. 36.

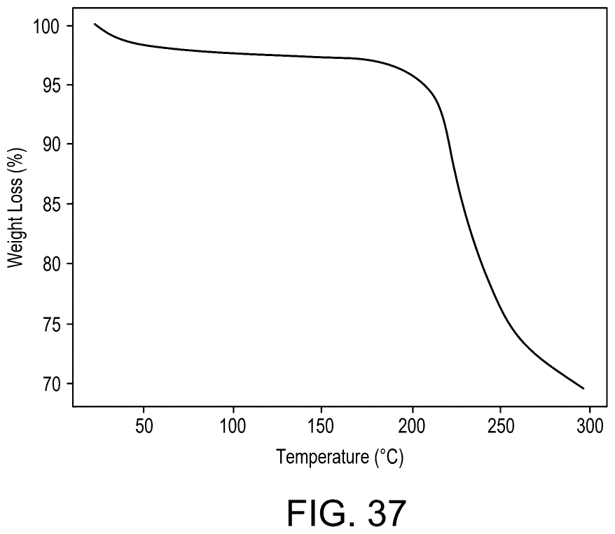

In some embodiments, the disclosure provides Form C1 characterized by a thermogravimetric analysis (TGA) thermograph substantially as shown in FIG. 37.

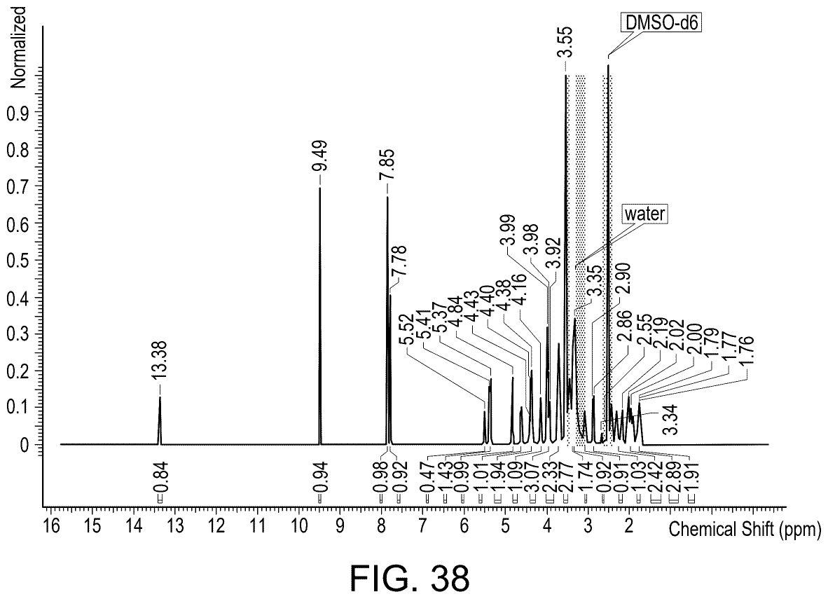

In some embodiments, the disclosure provides Form C1 characterized by a ¹H NMR spectrum substantially as shown in FIG. 38.

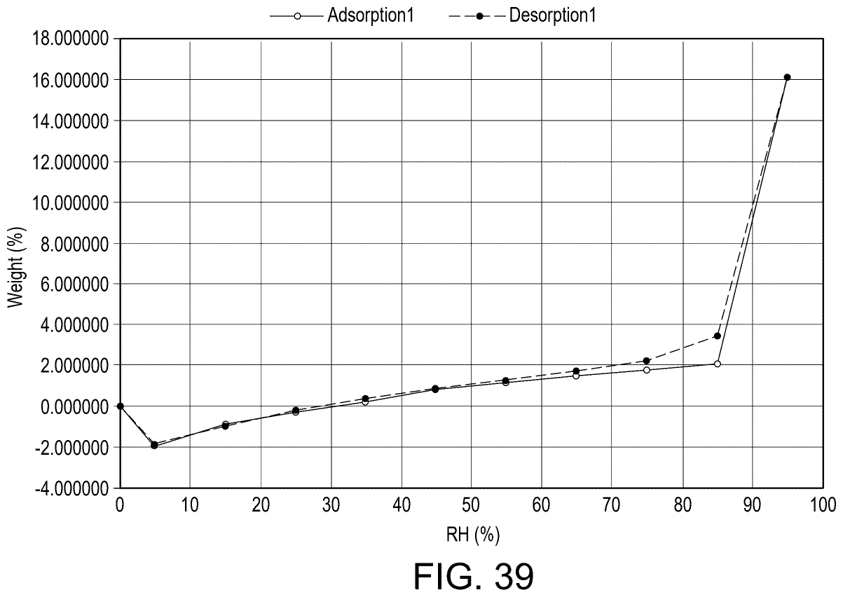

In some embodiments, the disclosure provides Form C1 characterized by a dynamic vapor sorption (DVS) graph substantially as shown in FIG. 39.

### Compound A Acetazolamide Co-Crystal (“Form C-E”)

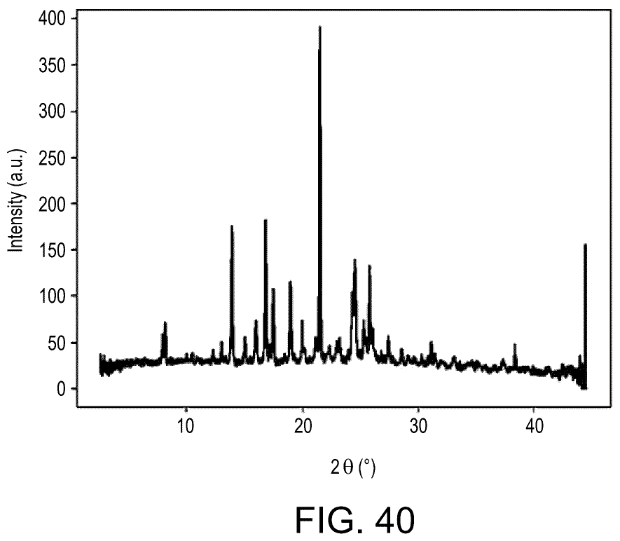

In some embodiments, the disclosure provides Compound A acetazolamide co-crystal (“Form C-E”), characterized by an X-ray powder diffraction (XRPD) pattern comprising peaks at 7.0, 5.0, and 23.2±0.2° 2θ using CuKα radiation. In some embodiments, Form C-E is further characterized by a peak at 19.7±0.2° 2θ using CuKα radiation. In some embodiments, Form C-E is also further characterized by a peak at 17.9±0.2° 2θ using CuKα radiation. In some embodiments, Form C-E is also further characterized by peaks at 18.7, 17.0, 6.3, 19.1, 24.9, 20.3, 22.8, 23.8, and 21.4±0.2° 2θ using CuKα radiation. In some embodiments, the disclosure provides Form C-E characterized by an X-ray powder diffraction (XRPD) pattern substantially as shown in FIG. 40.

### Compound A L-Tartaric Acid Co-Crystal (“Form C-D”)

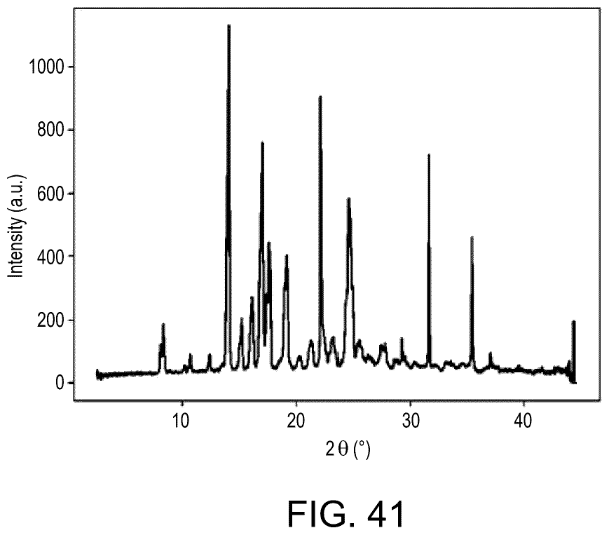

In some embodiments, the disclosure provides Compound A L-tartaric acid co-crystal (“Form C-D”), characterized by an X-ray powder diffraction (XRPD) pattern comprising peaks at 14.1, 22.2, and 17.1±0.2° 2θ using CuKα radiation. In some embodiments, Form C-D is further characterized by a peak at 31.7±0.2° 2θ using CuKα radiation. In some embodiments, Form C-D is also further characterized by a peak at 24.6±0.2° 2θ using CuKα radiation. In some embodiments, Form C-D is also further characterized by peaks at 35.5, 17.7, and 19.3±0.2° 2θ using CuKα radiation. In some embodiments, the disclosure provides Form C-D characterized by an X-ray powder diffraction (XRPD) pattern substantially as shown in FIG. 41.

### Compound A Glutaric Acid Co-Crystal (“Form C-C”)

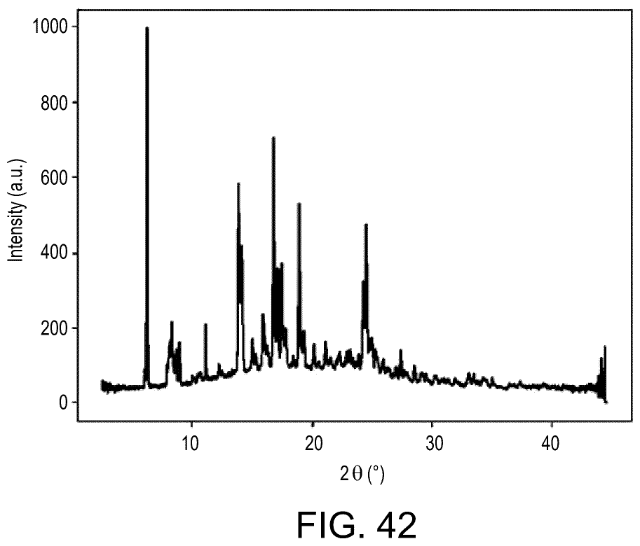

In some embodiments, the disclosure provides Compound A glutaric acid co-crystal (“Form C-C”), characterized by an X-ray powder diffraction (XRPD) pattern comprising peaks at 6.3, 16.8, and 13.9±0.2° 2θ using CuKα radiation. In some embodiments, Form C-C is further characterized by a peak at 19.0±0.2° 2θ using CuKα radiation. In some embodiments, Form C-C is also further characterized by a peak at 24.6±0.2° 2θ using CuKα radiation. In some embodiments. Form C-C is also further characterized by peaks at 14.2, 17.5, and 17.2±0.2° 2θ using CuKα radiation. In some embodiments, the disclosure provides Form C-C characterized by an X-ray powder diffraction (XRPD) pattern substantially as shown in FIG. 42.

### Solvate Forms of Compound A

In some embodiments, the disclosure provides solvate (e.g., hydrate) forms of Compound A. In some embodiments, the crystalline form of Compound A is a free base hydrate.

### Compound A Free Base Hydrate (“Form 1”)

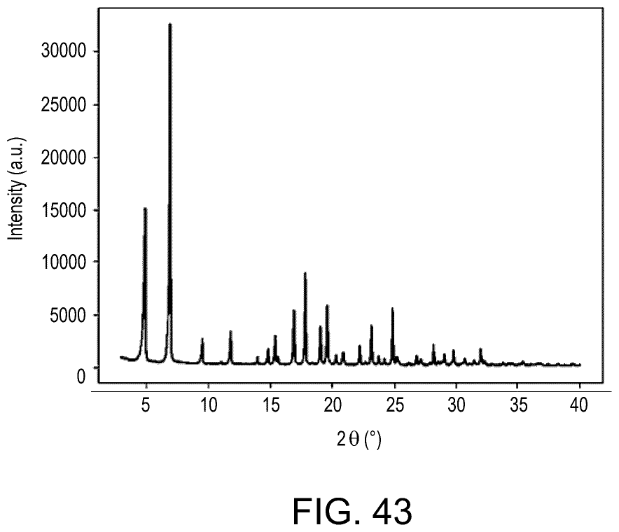

In some embodiments, the disclosure provides Compound A free base hydrate (“Form 1”), characterized by an X-ray powder diffraction (XRPD) pattern comprising peaks at 7.0, 5.0, and 17.9±0.2° 2θ using CuKα radiation. In some embodiments, Form 1 is further characterized by a peak at 17.0±0.2° 2θ using CuKα radiation. In some embodiments, Form 1 is also further characterized by a peak at 19.7±0.2° 2θ using CuKα radiation. In some embodiments, the disclosure provides Form 1 characterized by an X-ray powder diffraction (XRPD) pattern substantially as shown in FIG. 43.

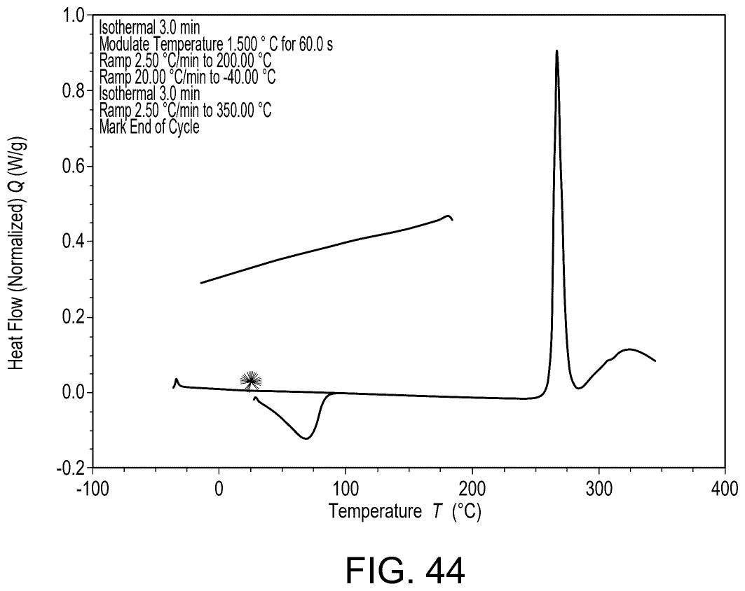

In some embodiments, the disclosure provides Form 1 characterized by a differential scanning calorimetry (DSC) thermograph substantially as shown in FIG. 44.

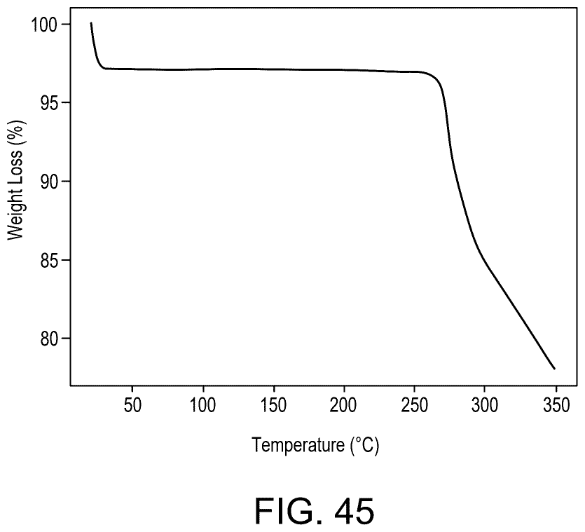

In some embodiments, the disclosure provides Form 1 characterized by a thermogravimetric analysis (TGA) thermograph substantially as shown in FIG. 45.

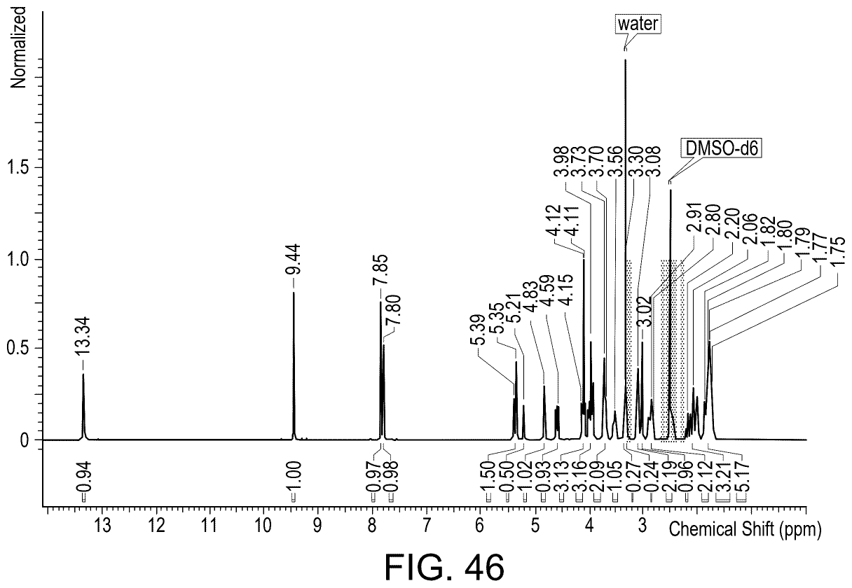

In some embodiments, the disclosure provides Form 1 characterized by a ¹H NMR spectrum substantially as shown in FIG. 46.

### Compound A Free Base Hydrate (“Form 2”)

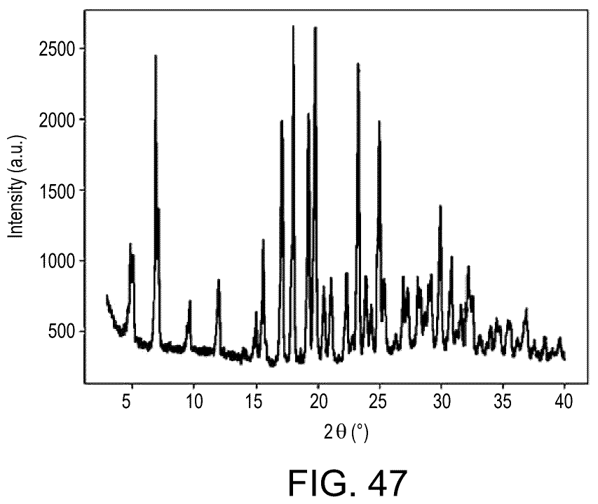

In some embodiments, the disclosure provides Compound A free base hydrate (“Form 2”), characterized by an X-ray powder diffraction (XRPD) pattern comprising peaks at 18.1, 19.9, and 7.0±0.2° 2θ using CuKα radiation. In some embodiments, Form 2 is further characterized by a peak at 23.4±0.2° 2θ using CuKα radiation. In some embodiments, Form 2 is also further characterized by a peak at 19.3±0.2° 2θ using CuKα radiation. In some embodiments, Form 2 is also further characterized by peaks at 17.2, 25.1, 30.0, 7.2, 15.6, and 30.9±0.2° 2θ using CuKα radiation. In some embodiments, the disclosure provides Form 2 characterized by an X-ray powder diffraction (XRPD) pattern substantially as shown in FIG. 47.

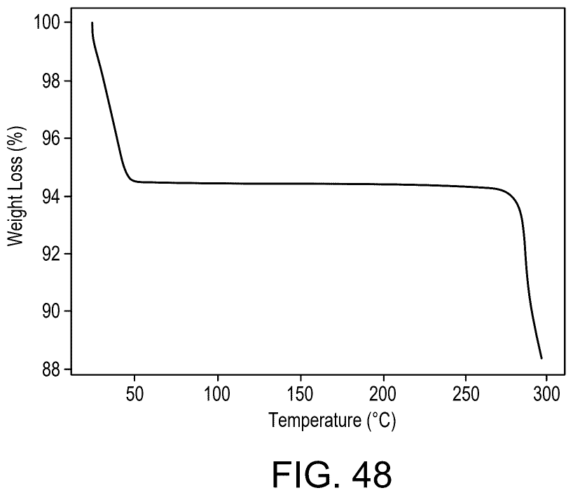

In some embodiments, the disclosure provides Form 2 characterized by a thermogravimetric analysis (TGA) thermograph substantially as shown in FIG. 48.

### Amorphous Forms of Compound A

### Amorphous Compound A Oxalate (“Form A1”)

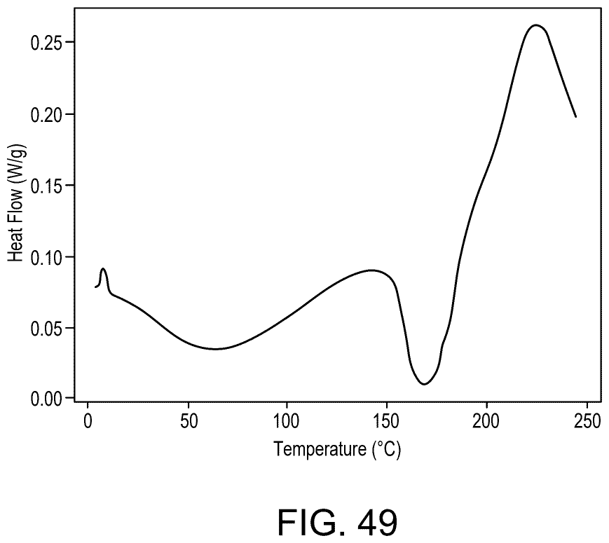

In some embodiments, the disclosure provides amorphous Compound A oxalate (“Form A1”), characterized by a differential scanning calorimetry (DSC) thermograph substantially as shown in FIG. 49.

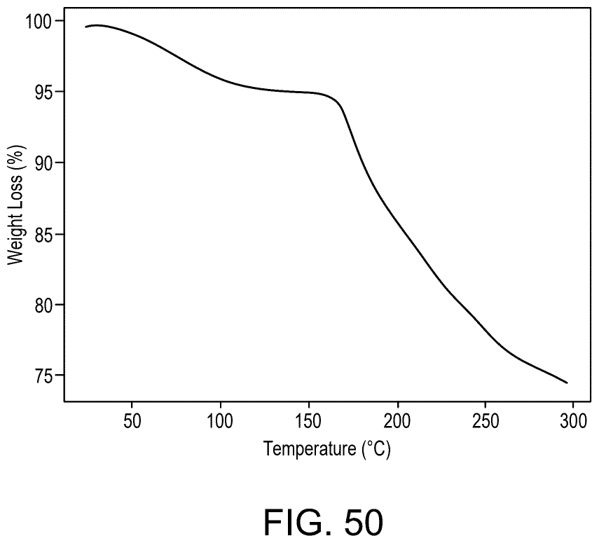

In some embodiments, the disclosure provides Form A1 characterized by a thermogravimetric analysis (TGA) thermograph substantially as shown in FIG. 50.

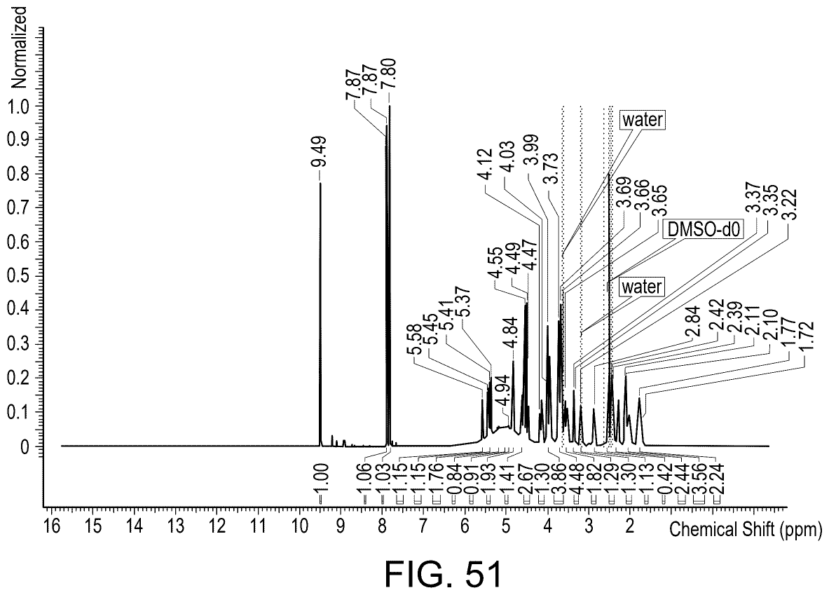

In some embodiments, the disclosure provides Form A1 characterized by a ¹H NMR spectrum substantially as shown in FIG. 51.

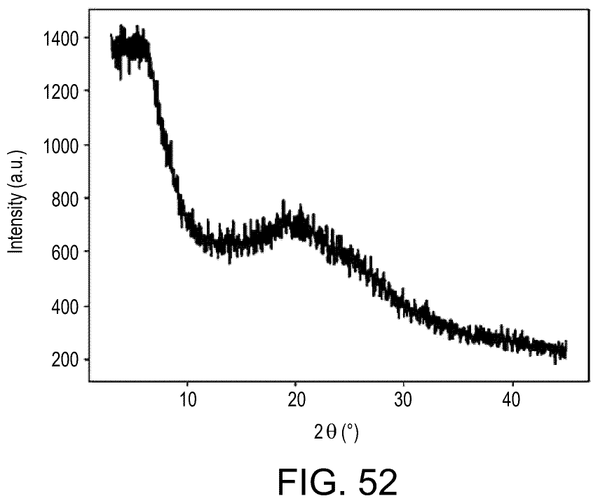

In some embodiments, the disclosure provides Form A1 characterized by an X-ray powder diffraction (XRPD) pattern substantially as shown in FIG. 52.

### Amorphous Compound A Tosylate (“Form A1-A”)

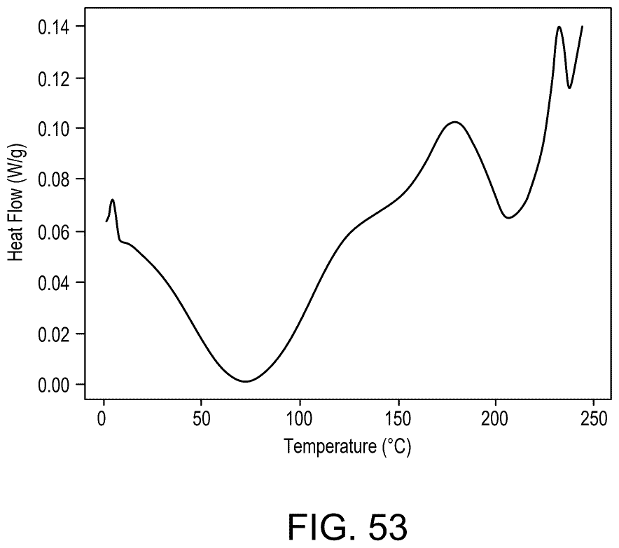

In some embodiments, the disclosure provides amorphous Compound A tosylate (“Form A1-A”), characterized by a differential scanning calorimetry (DSC) thermograph substantially as shown in FIG. 53.

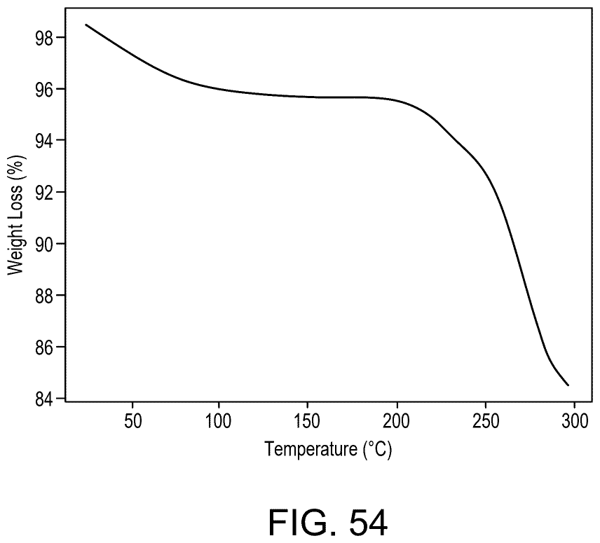

In some embodiments, the disclosure provides Form A1-A characterized by a thermogravimetric analysis (TGA) thermograph substantially as shown in FIG. 54.

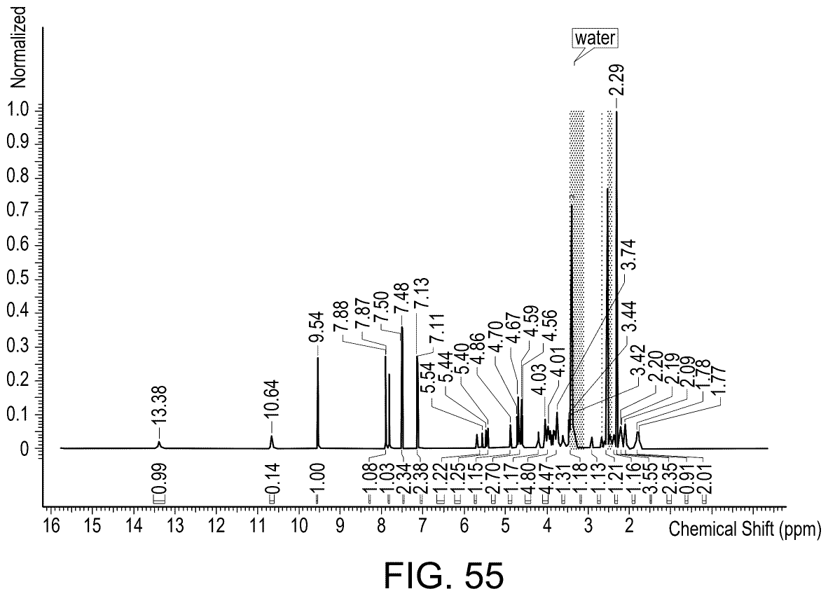

In some embodiments, the disclosure provides Form A1-A characterized by a ¹H NMR spectrum substantially as shown in FIG. 55.

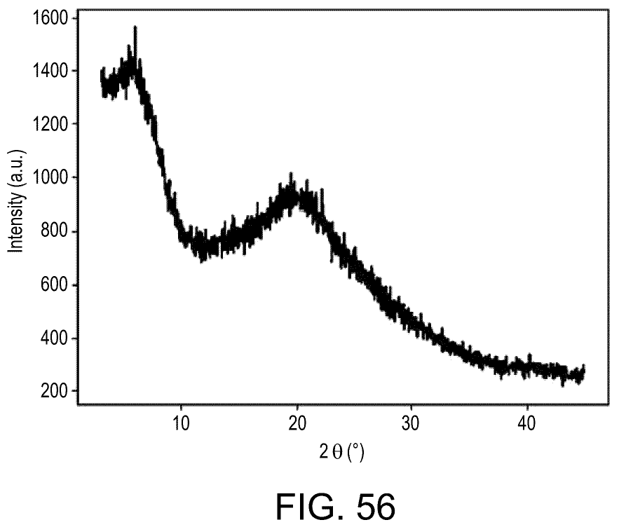

In some embodiments, the disclosure provides Form A1-A characterized by an X-ray powder diffraction (XRPD) pattern substantially as shown in FIG. 56.

### Amorphous Compound A Saccharinate (“Form A1-B”)

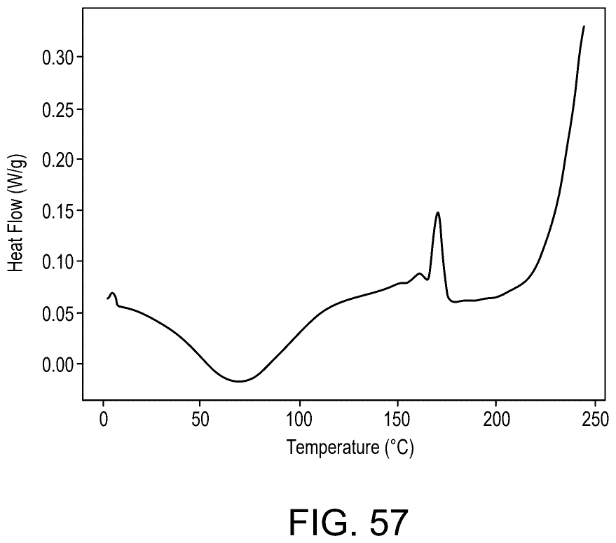

In some embodiments, the disclosure provides amorphous Compound A saccharinate (“Form A1-B”), characterized by a differential scanning calorimetry (DSC) thermograph substantially as shown in FIG. 57.

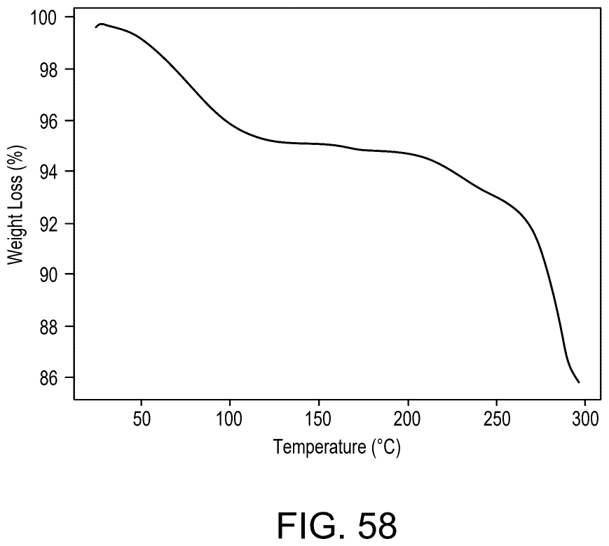

In some embodiments, the disclosure provides Form A1-B characterized by a thermogravimetric analysis (TGA) thermograph substantially as shown in FIG. 58.

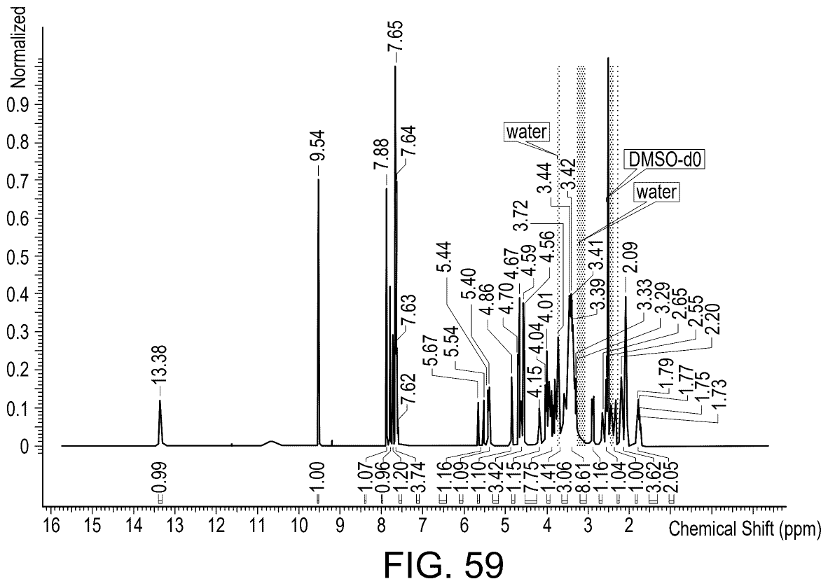

In some embodiments, the disclosure provides Form A1-B characterized by a ¹H NMR spectrum substantially as shown in FIG. 59.

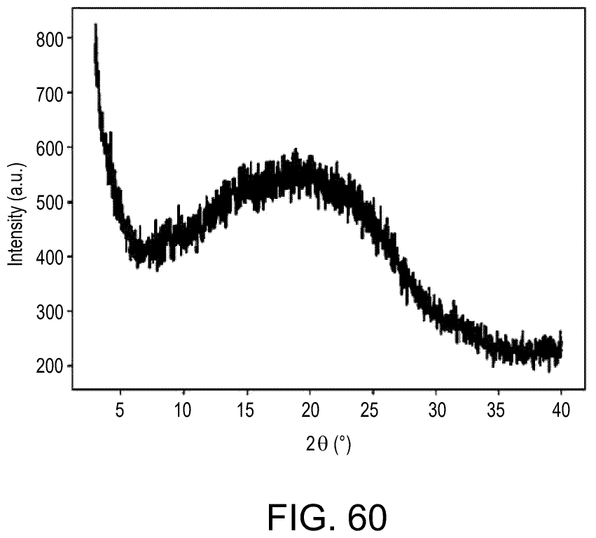

In some embodiments, the disclosure provides Form A1-B characterized by an X-ray powder diffraction (XRPD) pattern substantially as shown in FIG. 60.

### Amorphous Compound A Citrate (“Form A1-C”)

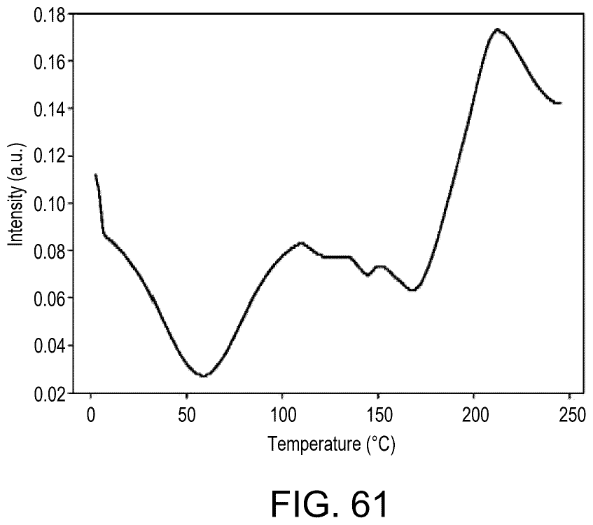

In some embodiments, the disclosure provides amorphous Compound A citrate (“Form A1-C”), characterized by a differential scanning calorimetry (DSC) thermograph substantially as shown in FIG. 61.

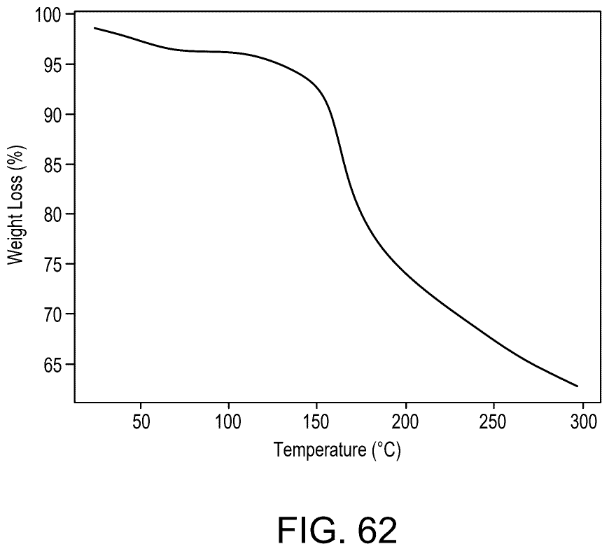

In some embodiments, the disclosure provides Form A1-C characterized by a thermogravimetric analysis (TGA) thermograph substantially as shown in FIG. 62.

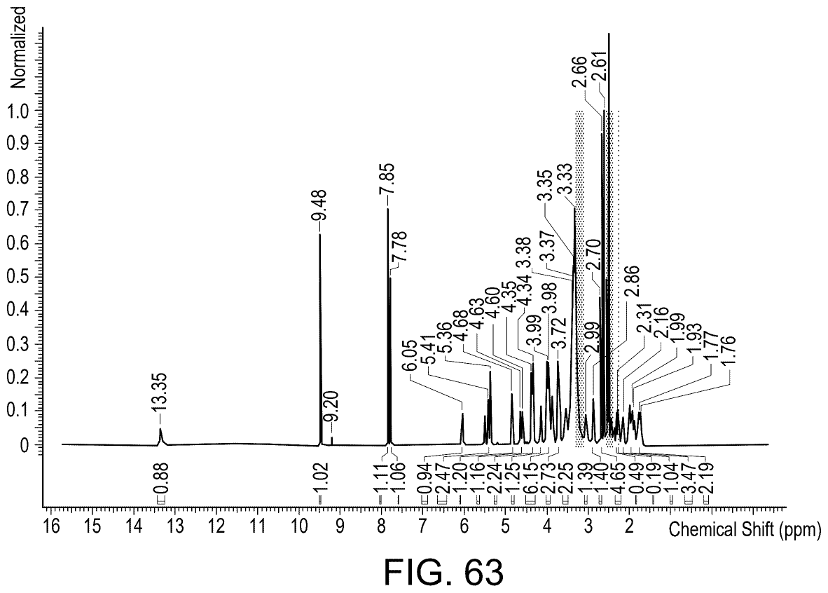

In some embodiments, the disclosure provides Form A1-C characterized by a ¹H NMR spectrum substantially as shown in FIG. 63.

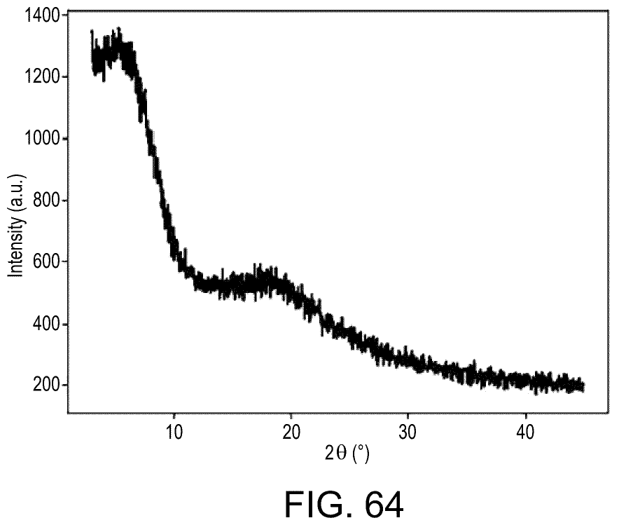

In some embodiments, the disclosure provides Form A1-C characterized by an X-ray powder diffraction (XRPD) pattern substantially as shown in FIG. 64.

### Amorphous Compound A Free Base (“Form A2”)

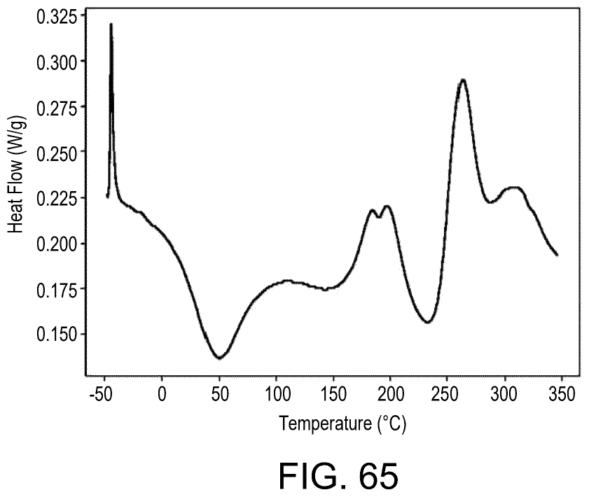

In some embodiments, the disclosure provides amorphous Compound A free base (“Form A2”), characterized by a differential scanning calorimetry (DSC) thermograph substantially as shown in FIG. 65.

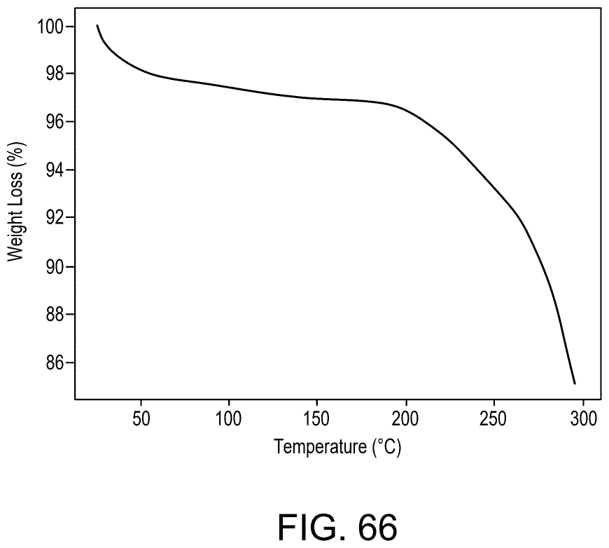

In some embodiments, the disclosure provides Form A2 characterized by a thermogravimetric analysis (TGA) thermograph substantially as shown in FIG. 66.

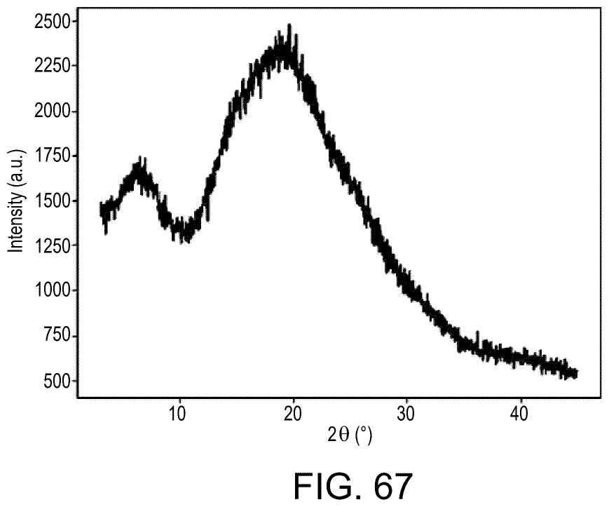

In some embodiments, the disclosure provides Form A2 characterized by an X-ray powder diffraction (XRPD) pattern substantially as shown in FIG. 67.

### Amorphous Compound A Besylate (“Form A1-D”)

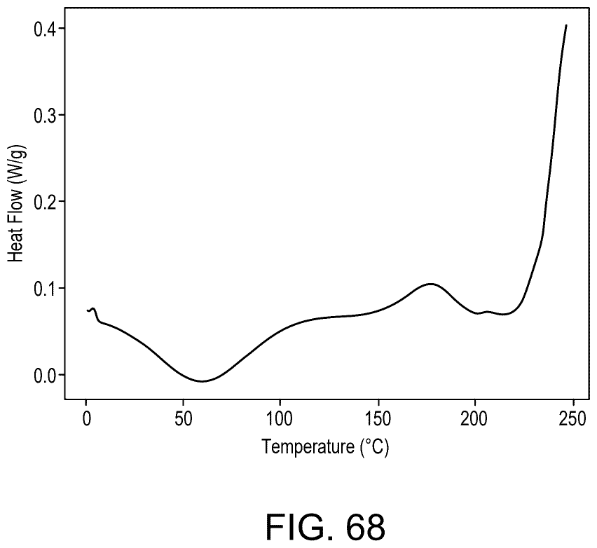

In some embodiments, the disclosure provides amorphous Compound A besylate (“Form A1-D”), characterized by a differential scanning calorimetry (DSC) thermograph substantially as shown in FIG. 68.

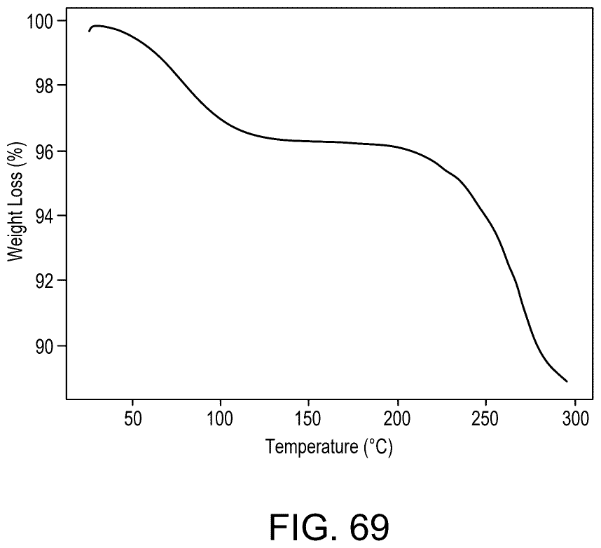

In some embodiments, the disclosure provides Form A1-D characterized by a thermogravimetric analysis (TGA) thermograph substantially as shown in FIG. 69.

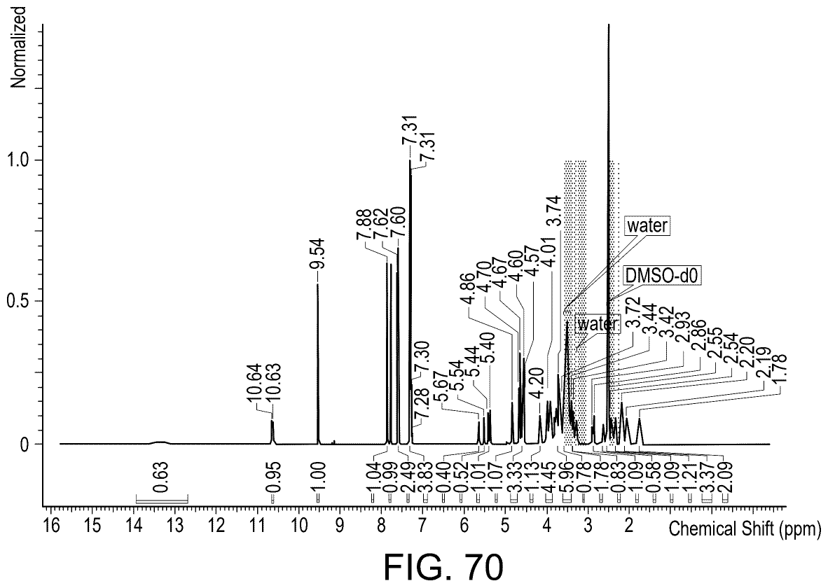

In some embodiments, the disclosure provides Form A1-D characterized by a ¹H NMR spectrum substantially as shown in FIG. 70.

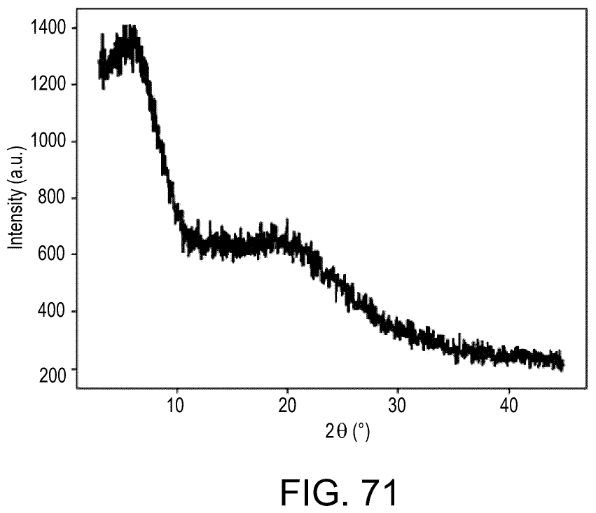

In some embodiments, the disclosure provides Form A1-D characterized by an X-ray powder diffraction (XRPD) pattern substantially as shown in FIG. 71.

### Amorphous Compound A Esylate (“Form A1-E”)

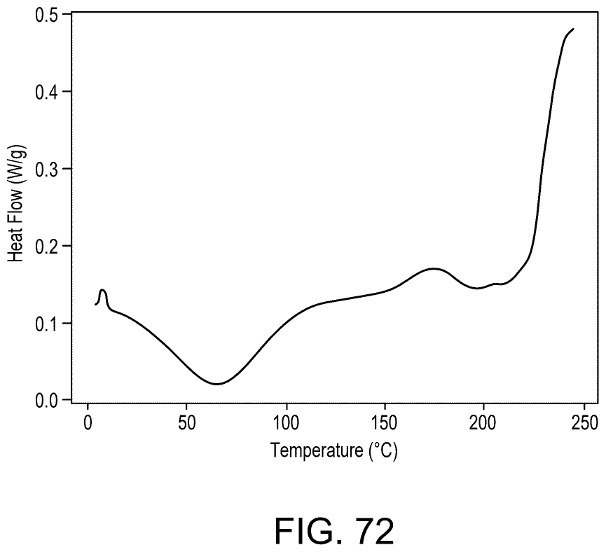

In some embodiments, the disclosure provides amorphous Compound A esylate (“Form A1-E”), characterized by a differential scanning calorimetry (DSC) thermograph substantially as shown in FIG. 72.

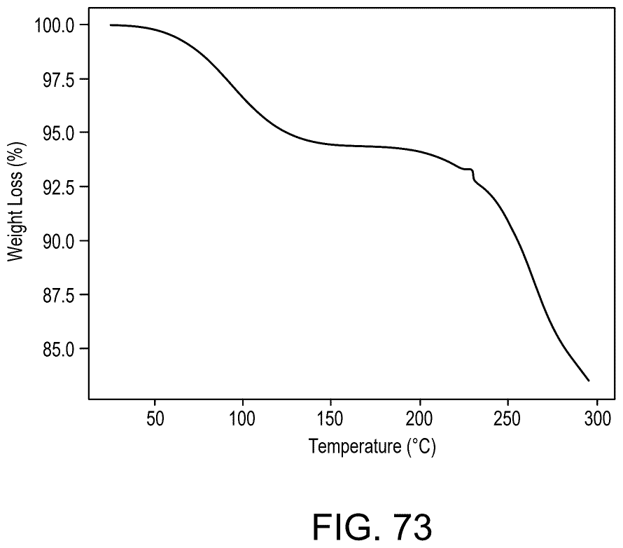

In some embodiments, the disclosure provides Form A1-E characterized by a thermogravimetric analysis (TGA) thermograph substantially as shown in FIG. 73.

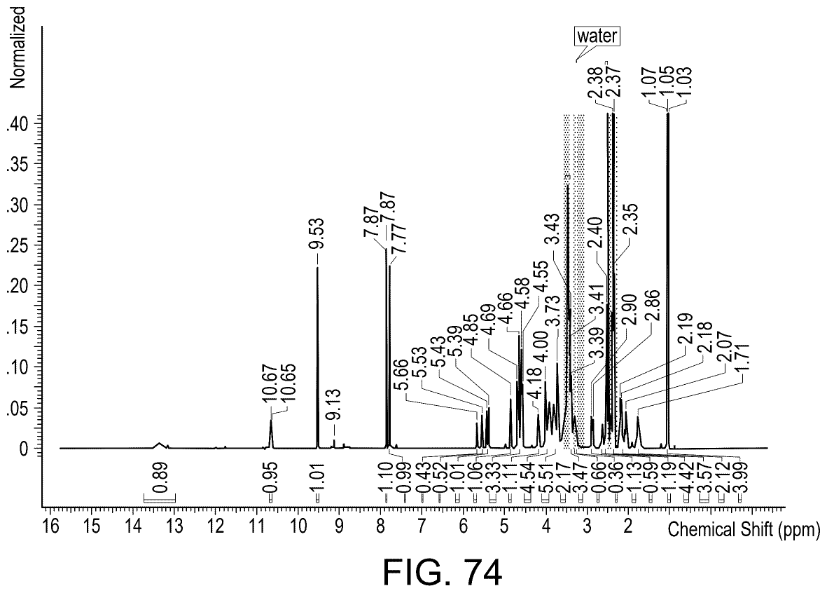

In some embodiments, the disclosure provides Form A1-E characterized by a ¹H NMR spectrum substantially as shown in FIG. 74.

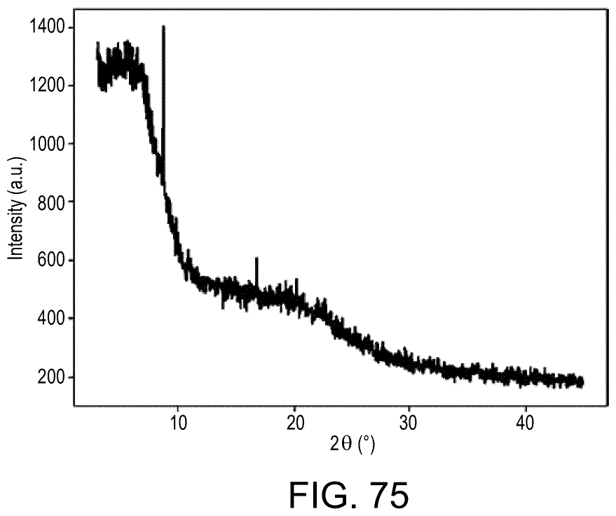

In some embodiments, the disclosure provides Form A1-E characterized by an X-ray powder diffraction (XRPD) pattern substantially as shown in FIG. 75.

### Pharmaceutical Compositions

The disclosure also provides pharmaceutical compositions comprising the disclosed solid forms of Compound A. In some embodiments, the disclosure provides pharmaceutical compositions comprising the salts, crystalline forms, or amorphous forms disclosed herein and at least one pharmaceutically acceptable excipient.

The pharmaceutical composition can be formulated for particular routes of administration such as oral administration, parenteral administration, and rectal administration, etc. The pharmaceutical compositions can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

The term “pharmaceutically acceptable excipient” as used herein refers to a broad range of ingredients that may be combined with a compound or salt disclosed herein to prepare a pharmaceutical composition or formulation. Typically, excipients include, but are not limited to, diluents, colorants, vehicles, anti-adherants, glidants, disintegrants, flavoring agents, coatings, binders, sweeteners, lubricants, sorbents, preservatives, and the like.

### Methods of Treatment

The disclosure also provides methods of using the disclosed solid forms and pharmaceutical compositions.

In some embodiments, the disclosure provides methods of treating a disease or disorder responsive to inhibition of certain mutated forms of KRAS (e.g., KRAS G12C, G12D, G12V, G12A, G12S, G13D, Q61H, and/or Q61L).

In some embodiments, the disclosure provides methods of treating a cancer in a subject in need thereof, comprising administering to the subject a therapeutically effective amount of the disclosed salts, crystalline forms, or amorphous forms or pharmaceutical compositions comprising the disclosed solid forms.

In some embodiments, the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

In some embodiments, the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma. In some embodiments, the cancer is non-small cell lung cancer. In some embodiments, the cancer is colorectal cancer. In some embodiments, the cancer is pancreatic cancer.

Cancers with KRAS Mutations

Provided herein are methods of treating a cancer comprising a KRAS mutation in a subject in need thereof comprising administering to the subject a therapeutically effective amount of a combination disclosed herein.

In certain embodiments, the cancer is a cancer comprising a KRAS G12D, G12V, G12A, G12S, G12R, G13D, Q61H, Q61L, Q61R or G12C mutation. In some embodiments, the cancer is a cancer comprising a KRAS G12D mutation. In some embodiments, the cancer is a cancer comprising a KRAS G12V mutation. In some embodiments, the cancer is a cancer comprising a KRAS G12C mutation. In some embodiments, the cancer is a cancer comprising a KRAS G12A mutation. In some embodiments, the cancer is a cancer comprising a KRAS G12S mutation. In some embodiments, the cancer is a cancer comprising a KRAS G12R mutation. In some embodiments, the cancer is a cancer comprising a KRAS G13D mutation. In some embodiments, the cancer is a cancer comprising a KRAS Q61H mutation. In some embodiments, the cancer is a cancer comprising a KRAS Q61L mutation. In some embodiments, the cancer is a cancer comprising a KRAS Q61R mutation. In some embodiments, the cancer is a cancer comprising a KRAS WT amplified mutation.

In some cases, the cancer disclosed herein is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma. In some cases, the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma. In some cases, the cancer is non-small cell lung cancer. In some cases, the cancer is colorectal cancer. In some cases, the cancer is pancreatic cancer. In some cases, the cancer is gastric cancer. In some cases, the cancer is esophageal cancer. In some cases, the cancer is heptobilliary cancer. In some cases, the cancer is ovarian cancer. In some cases, the cancer is endometrial cancer.

In certain embodiments, the cancer is pancreatic cancer comprising a KRAS G12D mutation. In certain embodiments, the cancer is pancreatic cancer comprising a KRAS G12V mutation. In certain embodiments, the cancer is pancreatic cancer comprising a KRAS G12C mutation. In certain embodiments, the cancer is pancreatic cancer comprising a KRAS G12R mutation. In certain embodiments, the cancer is pancreatic cancer comprising a KRAS G12A mutation. In certain embodiments, the cancer is pancreatic cancer comprising a KRAS G12S mutation. In certain embodiments, the cancer is pancreatic cancer comprising a KRAS G13D mutation. In certain embodiments, the cancer is pancreatic cancer comprising a KRAS Q61H mutation. In certain embodiments, the cancer is pancreatic cancer comprising a KRAS Q61L mutation. In certain embodiments, the cancer is pancreatic cancer comprising a KRAS Q61R mutation. In certain embodiments, the cancer is pancreatic cancer comprising a KRAS WT amplified mutation. In certain embodiments, the pancreatic cancer is pancreatic ductal adenocarcinoma (PDAC).

In certain embodiments, the pancreatic ductal adenocarcinoma (PDAC) is PDAC comprising a KRAS G12D mutation. In certain embodiments, the pancreatic ductal adenocarcinoma (PDAC) is PDAC comprising a KRAS G12V mutation. In certain embodiments, the pancreatic ductal adenocarcinoma (PDAC) is PDAC comprising a KRAS G12C mutation. In certain embodiments, the pancreatic ductal adenocarcinoma (PDAC) is PDAC comprising a KRAS G12R mutation. In certain embodiments, the pancreatic ductal adenocarcinoma (PDAC) is PDAC comprising a KRAS G12A mutation. In certain embodiments, the pancreatic ductal adenocarcinoma (PDAC) is PDAC comprising a KRAS G12S mutation. In certain embodiments, the pancreatic ductal adenocarcinoma (PDAC) is PDAC comprising a KRAS G13D mutation. In certain embodiments, the pancreatic ductal adenocarcinoma (PDAC) is PDAC comprising a KRAS Q61H mutation. In certain embodiments, the pancreatic ductal adenocarcinoma (PDAC) is PDAC comprising a KRAS Q61L mutation. In certain embodiments, the pancreatic ductal adenocarcinoma (PDAC) is PDAC comprising a KRAS Q61R mutation. In certain embodiments, the pancreatic ductal adenocarcinoma (PDAC) is PDAC comprising a KRAS WT amplified mutation.

In certain embodiments, the cancer is colorectal cancer comprising a KRAS G12D mutation. In certain embodiments, the cancer is colorectal cancer comprising a KRAS G12V mutation. In certain embodiments, the cancer is colorectal cancer comprising a KRAS G12C mutation. In certain embodiments, the cancer is colorectal cancer comprising a KRAS G12R mutation. In certain embodiments, the cancer is colorectal cancer comprising a KRAS G12A mutation. In certain embodiments, the cancer is colorectal cancer comprising a KRAS G12S mutation. In certain embodiments, the cancer is colorectal cancer comprising a KRAS G13D mutation. In certain embodiments, the cancer is colorectal cancer comprising a KRAS Q61H mutation. In certain embodiments, the cancer is colorectal cancer comprising a KRAS Q61L mutation. In certain embodiments, the cancer is colorectal cancer comprising a KRAS Q61R mutation. In certain embodiments, the cancer is colorectal cancer comprising a KRAS WT amplified mutation.

In certain embodiments, the cancer is non-small cell lung cancer comprising a KRAS G12D mutation. In certain embodiments, the cancer is non-small cell lung cancer comprising a KRAS G12V mutation. In certain embodiments, the cancer is non-small cell lung cancer comprising a KRAS G12C mutation. In certain embodiments, the cancer is non-small cell lung cancer comprising a KRAS G12R mutation. In certain embodiments, the cancer is non-small cell lung cancer comprising a KRAS G12A mutation. In certain embodiments, the cancer is non-small cell lung cancer comprising a KRAS G12S mutation. In certain embodiments, the cancer is non-small cell lung cancer comprising a KRAS G13D mutation. In certain embodiments, the cancer is non-small cell lung cancer comprising a KRAS Q61H mutation. In certain embodiments, the cancer is non-small cell lung cancer comprising a KRAS Q61L mutation. In certain embodiments, the cancer is non-small cell lung cancer comprising a KRAS Q61R mutation. In certain embodiments, the cancer is non-small cell lung cancer comprising a KRAS WT amplified mutation.

In certain embodiments, the non-small cell lung cancer is lung adenocarcinoma (LUAD). In certain embodiments, the cancer is lung adenocarcinoma (LUAD) comprising a KRAS G12D mutation. In certain embodiments, the cancer is lung adenocarcinoma (LUAD) comprising a KRAS G12V mutation. In certain embodiments, the cancer is lung adenocarcinoma (LUAD) comprising a KRAS G12C mutation. In certain embodiments, the cancer is lung adenocarcinoma (LUAD) comprising a KRAS G12R mutation. In certain embodiments, the cancer is lung adenocarcinoma (LUAD) comprising a KRAS G12A mutation. In certain embodiments, the cancer is lung adenocarcinoma (LUAD) comprising a KRAS G12S mutation. In certain embodiments, the cancer is lung adenocarcinoma (LUAD) comprising a KRAS G13D mutation. In certain embodiments, the cancer is lung adenocarcinoma (LUAD) comprising a KRAS Q61H mutation. In certain embodiments, the cancer is lung adenocarcinoma (LUAD) comprising a KRAS Q61L mutation. In certain embodiments, the cancer is lung adenocarcinoma (LUAD) comprising a KRAS Q61R mutation. In certain embodiments, the cancer is lung adenocarcinoma (LUAD) comprising a KRAS WT amplified mutation.

In certain embodiments, the cancer is gastric cancer comprising a KRAS G12D mutation. In certain embodiments, the cancer is gastric cancer comprising a KRAS G12V mutation. In certain embodiments, the cancer is gastric cancer comprising a KRAS G12C mutation. In certain embodiments, the cancer is gastric cancer comprising a KRAS G12R mutation. In certain embodiments, the cancer is gastric cancer comprising a KRAS G12A mutation. In certain embodiments, the cancer is gastric cancer comprising a KRAS G12S mutation. In certain embodiments, the cancer is gastric cancer comprising a KRAS G13D mutation. In certain embodiments, the cancer is gastric cancer comprising a KRAS Q61H mutation. In certain embodiments, the cancer is gastric cancer comprising a KRAS Q61L mutation. In certain embodiments, the cancer is gastric cancer comprising a KRAS Q61R mutation. In certain embodiments, the cancer is gastric cancer comprising a KRAS WT amplified mutation.

In certain embodiments, the cancer is esophageal cancer comprising a KRAS G12D mutation. In certain embodiments, the cancer is esophageal cancer comprising a KRAS G12V mutation. In certain embodiments, the cancer is esophageal cancer comprising a KRAS G12C mutation. In certain embodiments, the cancer is esophageal cancer comprising a KRAS G12R mutation. In certain embodiments, the cancer is esophageal cancer comprising a KRAS G12A mutation. In certain embodiments, the cancer is esophageal cancer comprising a KRAS G12S mutation. In certain embodiments, the cancer is esophageal cancer comprising a KRAS G13D mutation. In certain embodiments, the cancer is esophageal cancer comprising a KRAS Q61H mutation. In certain embodiments, the cancer is esophageal cancer comprising a KRAS Q61L mutation. In certain embodiments, the cancer is esophageal cancer comprising a KRAS Q61R mutation. In certain embodiments, the cancer is esophageal cancer comprising a KRAS WT amplified mutation.

In certain embodiments, the cancer is heptobiliary cancer comprising a KRAS G12D mutation. In certain embodiments, the cancer is heptobiliary cancer comprising a KRAS G12V mutation. In certain embodiments, the cancer is heptobilliary cancer comprising a KRAS G12C mutation. In certain embodiments, the cancer is heptobiliary cancer comprising a KRAS G12R mutation. In certain embodiments, the cancer is heptobiliary cancer comprising a KRAS G12A mutation. In certain embodiments, the cancer is heptobiliary cancer comprising a KRAS G12S mutation. In certain embodiments, the cancer is heptobilliary cancer comprising a KRAS G13D mutation. In certain embodiments, the cancer is heptobilliary cancer comprising a KRAS Q61H mutation. In certain embodiments, the cancer is heptobilliary cancer comprising a KRAS Q61L mutation. In certain embodiments, the cancer is heptobilliary cancer comprising a KRAS Q61R mutation. In certain embodiments, the cancer is heptobilliary cancer comprising a KRAS WT amplified mutation.

In certain embodiments, the cancer is ovarian cancer comprising a KRAS G12D mutation. In certain embodiments, the cancer is ovarian cancer comprising a KRAS G12V mutation. In certain embodiments, the cancer is ovarian cancer comprising a KRAS G12C mutation. In certain embodiments, the cancer is ovarian cancer comprising a KRAS G12R mutation. In certain embodiments, the cancer is ovarian cancer comprising a KRAS G12A mutation. In certain embodiments, the cancer is ovarian cancer comprising a KRAS G12S mutation. In certain embodiments, the cancer is ovarian cancer comprising a KRAS G13D mutation. In certain embodiments, the cancer is ovarian cancer comprising a KRAS Q61H mutation. In certain embodiments, the cancer is ovarian cancer comprising a KRAS Q61L mutation. In certain embodiments, the cancer is ovarian cancer comprising a KRAS Q61R mutation. In certain embodiments, the cancer is ovarian cancer comprising a KRAS WT amplified mutation.

In certain embodiments, the cancer is endometrial cancer comprising a KRAS G12D mutation. In certain embodiments, the cancer is endometrial cancer comprising a KRAS G12V mutation. In certain embodiments, the cancer is endometrial cancer comprising a KRAS G12C mutation. In certain embodiments, the cancer is endometrial cancer comprising a KRAS G12R mutation. In certain embodiments, the cancer is endometrial cancer comprising a KRAS G12A mutation. In certain embodiments, the cancer is endometrial cancer comprising a KRAS G12S mutation. In certain embodiments, the cancer is endometrial cancer comprising a KRAS G13D mutation. In certain embodiments, the cancer is endometrial cancer comprising a KRAS Q61H mutation. In certain embodiments, the cancer is endometrial cancer comprising a KRAS Q61L mutation. In certain embodiments, the cancer is endometrial cancer comprising a KRAS Q61R mutation. In certain embodiments, the cancer is endometrial cancer comprising a KRAS WT amplified mutation.

### EMBODIMENTS

Described herein as embodiment 1 is a salt of Compound A

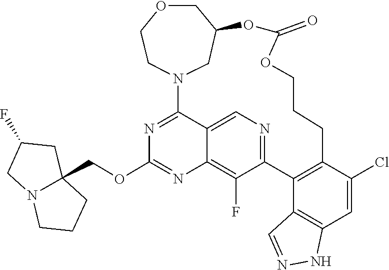 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

(Compound A), wherein the salt is a pharmaceutically acceptable salt.

Described herein as embodiment 2 is the salt of embodiment 1, wherein the pharmaceutically acceptable salt is selected from maleate, citrate, malonate, oxalate, tosylate, saccharinate, hydrobromide, hydrochloride, edisylate, besylate, esylate, hemiedisylate.

Described herein as embodiment 3 is the salt of embodiment 1 or 2, wherein the salt is crystalline, a hydrate, a solvate, or a combination thereof.

Described herein as embodiment 4 is the salt of any one of embodiments 1-3, wherein the salt is a malonate salt or maleate salt.

Described herein as embodiment 5 is the salt of any one of embodiments 1-4, wherein the salt is a malonate salt.

Described herein as embodiment 6 is the salt of any one of embodiments 1-5, wherein the salt is an anhydrous malonate salt.

Described herein as embodiment 7 is the salt of any one of embodiments 4-6, characterized by one or more of the following data selected from the group consisting of:
(a) one or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα;
(b) one or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm;
(c) a differential scanning calorimetry (DSC) thermogram comprising an endotherm with an onset of about 170° C.±1° C.; and
(d) a thermogravimetric analysis (TGA) thermogram comprising a weight loss of about 13.4% when heated from about 170° C. to about 190° C.

Described herein as embodiment 8 is the salt of embodiment 7, characterized by one or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 9 is the salt of embodiment 8, characterized by two or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 10 is the salt of embodiment 9, characterized by three or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 11 is the salt of embodiment 10, characterized by four or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 12 is the salt of embodiment 11, characterized by five or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 13 is the salt of embodiment 12, characterized by seven or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 14 is the salt of embodiment 13, characterized by ten or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

Described herein as embodiment 15 is the salt of any of embodiments 1-6, wherein the salt is a malonate salt of Compound A (“Form 1E”), characterized by an X-ray powder diffraction (XRPD) pattern comprising a peak at 16.7±0.2° 2θ using CuKα radiation.

Described herein as embodiment 16 is the salt of any of embodiments 1-6, wherein the salt is a malonate salt of Compound A (“Form 1E”), characterized by an X-ray powder diffraction (XRPD) pattern comprising a peak at 16.7 and 22.9±0.2° 2θ using CuKα radiation.

Described herein as embodiment 17 is the salt of any one of embodiments 1-6, wherein the salt is a malonate salt of Compound A (“Form 1E”), characterized by an X-ray powder diffraction (XRPD) pattern comprising a peak at 16.7, 22.9, and 18.6±0.2° 2θ using CuKα radiation.

Described herein as embodiment 18 is the salt of embodiment 17, further characterized by a peak at 19.4±0.2° 2θ using CuKα radiation.

Described herein as embodiment 19 is the salt of embodiment 18, further characterized by a peak at 17.7±0.2° 2θ using CuKα radiation.

Described herein as embodiment 20 is the salt of embodiment 19, further characterized by peaks at 24.6, 15.2, 23.3, and 28.0±0.2° 2θ using CuKα radiation.

Described herein as embodiment 21 is the salt of any one of embodiments 5-20, characterized by an X-ray powder diffraction (XRPD) pattern substantially as shown in FIG. 2.

Described herein as embodiment 22 is the salt of any one of embodiments 5-21, characterized by a differential scanning calorimetry (DSC) thermogram comprising an endotherm with an onset of about 170° C.±1° C.

Described herein as embodiment 23 is the salt of embodiment 22, characterized by a differential scanning calorimetry (DSC) thermograph substantially as shown in FIG. 3.

Described herein as embodiment 24 is the salt of any one of embodiments 5-22, characterized by a thermogravimetric analysis (TGA) thermogram comprising a weight loss of about 13.4% when heated from about 170° C. to about 190° C.

Described herein as embodiment 25 is the salt of embodiment 24, characterized by a thermogravimetric analysis (TGA) thermograph substantially as shown in FIG. 4.

Described herein as embodiment 26 is the salt of embodiment 7, characterized by one or more ¹³C ssNMR spectrum peaks selected from: 176.4, 175.9, 163.2, 161.6, 154.5, 147.9, 131.0, 121.8, 118.4, 110.6, 92.7, 80.4, and 78.7, ±0.2 ppm.

Described herein as embodiment 27 is the salt of embodiment 26, characterized by two or more ¹³C ssNMR spectrum peaks selected from: 176.4, 175.9, 163.2, 161.6, 154.5, 147.9, 131.0, 121.8, 118.4, 110.6, 92.7, 80.4, and 78.7, ±0.2 ppm.

Described herein as embodiment 28 is the salt of embodiment 27, characterized by three or more ¹³C ssNMR spectrum peaks selected from: 176.4, 175.9, 163.2, 161.6, 154.5, 147.9, 131.0, 121.8, 118.4, 110.6, 92.7, 80.4, and 78.7, ±0.2 ppm.

Described herein as embodiment 29 is the salt of embodiment 28, characterized by four or more ¹³C ssNMR spectrum peaks selected from: 176.4, 175.9, 163.2, 161.6, 154.5, 147.9, 131.0, 121.8, 118.4, 110.6, 92.7, 80.4, and 78.7, ±0.2 ppm.

Described herein as embodiment 30 is the salt of embodiment 29, characterized by five or more ¹³C ssNMR spectrum peaks selected from: 176.4, 175.9, 163.2, 161.6, 154.5, 147.9, 131.0, 121.8, 118.4, 110.6, 92.7, 80.4, and 78.7, ±0.2 ppm.

Described herein as embodiment 31 is the salt of any one of embodiments 7-30, characterized by a ¹³C ssNMR spectrum comprising a peak at 176.4±0.2 ppm.

Described herein as embodiment 32 is the salt of any one of embodiments 7-30, characterized by a ¹³C ssNMR spectrum comprising a peak at 92.7 and 176.4±0.2 ppm.

Described herein as embodiment 33 is the salt of any one of embodiments 7-30, characterized by a ¹³C ssNMR spectrum comprising a peak at 80.4, 92.7, and 176.4±0.2 ppm.

Described herein as embodiment 34 is the salt of embodiment 33, further characterized by a peak at 175.9±0.2 ppm.

Described herein as embodiment 35 is the salt of embodiment 34, further characterized by a peak at 147.9±0.2 ppm.

Described herein as embodiment 36 is the salt of embodiment 35, further characterized by peaks at 118.4, 121.8, and 154.5±0.2 ppm.

Described herein as embodiment 37 is the salt of embodiment 36, further characterized by peaks at 78.7, 110.6, 131.0, 161.6, and 163.2±0.2 ppm.

Described herein as embodiment 38 is the salt of any one of embodiments 7-37, characterized by a ¹³C ssNMR spectrum substantially as shown in FIG. 5A.

Described herein as embodiment 39 is the salt of any one of embodiments 7-38, characterized by a dynamic vapor sorption (DVS) graph substantially as shown in FIG. 6.

Described herein as embodiment 40 is the salt of any one of embodiments 1-4, wherein the salt is a maleate salt.

Described herein as embodiment 41 is the salt of embodiment 40, wherein the salt is a maleate salt hydrate.

Described herein as embodiment 42 is the salt of any one of embodiments 40-41, characterized by one or more of the following data selected from the group consisting of:
(a) one or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα;
(b) a differential scanning calorimetry (DSC) thermogram comprising an exotherm with an onset of about 170° C.±1° C.; and
(c) a thermogravimetric analysis (TGA) thermogram comprising a weight loss of about 7.2% when heated from about 170° C. to about 200° C.

Described herein as embodiment 43 is the salt of embodiment 42, characterized by one or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 44 is the salt of embodiment 43, characterized by two or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 45 is the salt of embodiment 44, characterized by three or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 46 is the salt of embodiment 45, characterized by four or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 47 is the salt of embodiment 46, characterized by five or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 48 is the salt of embodiment 47, characterized by seven or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 49 is the salt of embodiment 48, characterized by ten or more X-ray powder diffraction (XRPD) peaks selected from: 8.1, 8.5, 14.5, 15.3, 15.7, 16.7, 19.8, 20.5, 22.0, 25.4 and 26.7, ±0.2° 2θ using X-ray wavelength of 1.54 Å, wherein the source of radiation is CuKα.

Described herein as embodiment 50 is the salt of embodiment 42, where in the salt is a maleate salt of Compound A (“Form 1A”), characterized by an X-ray powder diffraction (XRPD) pattern comprising peaks at 8.5±0.2° 2θ using CuKα radiation.

Described herein as embodiment 51 is the salt of embodiment 50, wherein the salt is a maleate salt of Compound A (“Form 1A”), characterized by an X-ray powder diffraction (XRPD) pattern comprising peaks at 8.5 and 22.0±0.2° 2θ using CuKα radiation.

Described herein as embodiment 52 is the salt of embodiment 51, wherein the salt is a maleate salt of Compound A (“Form 1A”), characterized by an X-ray powder diffraction (XRPD) pattern comprising peaks at 8.5, 22.0, and 8.1±0.2° 2θ using CuKα radiation.

Described herein as embodiment 53 is the salt of embodiment 52, further characterized by a peak at 14.5±0.2° 2θ using CuKα radiation.

Described herein as embodiment 54 is the salt of embodiment 53, further characterized by a peak at 15.7±0.2° 2θ using CuKα radiation.

Described herein as embodiment 55 is the salt of embodiment 54, further characterized by peaks at 26.7, 20.5, 25.4, and 16.7±0.2° 2θ using CuKα radiation.

Described herein as embodiment 56 is the salt of any one of embodiments 40-55, characterized by an X-ray powder diffraction (XRPD) pattern substantially as shown in FIG. 7.

Described herein as embodiment 57 is the salt of any one of embodiments 40-56, characterized by a differential scanning calorimetry (DSC) thermogram comprising an exotherm with an onset of about 170° C.±1° C.

Described herein as embodiment 58 is the salt of embodiment 57, characterized by a differential scanning calorimetry (DSC) thermograph substantially as shown in FIG. 8.

Described herein as embodiment 59 is the salt of any one of embodiments 40-58, characterized by a thermogravimetric analysis (TGA) thermogram comprising a weight loss of about 7.2% when heated from about 170° C. to about 200° C.

Described herein as embodiment 60 is the salt of embodiment 59, characterized by a thermogravimetric analysis (TGA) thermograph substantially as shown in FIG. 9.

Described herein as embodiment 61 is the salt of any one of embodiments 40-60, characterized by a dynamic vapor sorption (DVS) graph substantially as shown in FIG. 11.

Described herein as embodiment 62 is the salt of any one of embodiments 1-3, wherein the salt is a citrate salt.

Described herein as embodiment 63 is the salt of embodiment 62, wherein the salt is a citrate salt hydrate.

Described herein as embodiment 64 is the salt of any one of embodiments 62-63, characterized by one or more of the following data selected from the group consisting of:
(a) one or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα;
(b) a differential scanning calorimetry (DSC) thermogram comprising an endotherm with an onset of about 150° C.±1° C.; and
(c) a thermogravimetric analysis (TGA) thermogram comprising a weight loss of about 29.8% when heated from about 150° C. to about 250° C.

Described herein as embodiment 65 is the salt of embodiment 64, characterized by one or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 66 is the salt of embodiment 65, characterized by two or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 67 is the salt of embodiment 66, characterized by three or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 68 is the salt of embodiment 67, characterized by four or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 69 is the salt of embodiment 68, characterized by five or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 70 is the salt of embodiment 69, characterized by seven or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 71 is the salt of embodiment 70, characterized by ten or more X-ray powder diffraction (XRPD) peaks selected from: 7.8, 12.8, 14.8, 15.5, 17.4, 18.5, 19.9, 21.3, 25.3, 26.6 and 29.2, ±0.2° 2θ using X-ray wavelength of 1.54 Å, wherein the source of radiation is CuKα.

Described herein as embodiment 72 is the salt of any one of embodiments 62-71, wherein the salt is a Compound A citrate salt hydrate (“Form 1B”), characterized by an X-ray powder diffraction (XRPD) pattern comprising peaks at 19.9±0.2° 2θ using CuKα radiation.

Described herein as embodiment 73 is the salt of any one of embodiments 62-71, wherein the salt is a Compound A citrate salt hydrate (“Form 1B”), characterized by an X-ray powder diffraction (XRPD) pattern comprising peaks at 19.9 and 12.8±0.2° 2θ using CuKα radiation.

Described herein as embodiment 74 is the salt of any one of embodiments 62-71, wherein the salt is a Compound A citrate salt hydrate (“Form 1B”), characterized by an X-ray powder diffraction (XRPD) pattern comprising peaks at 19.9, 12.8, and 18.5±0.2° 2θ using CuKα radiation.

Described herein as embodiment 75 is the salt of embodiment 74, further characterized by a peak at 7.8±0.2° 2θ using CuKα radiation.

Described herein as embodiment 76 is the salt of embodiment 75, further characterized by a peak at 17.4±0.2° 2θ using CuKα radiation.

Described herein as embodiment 77 is the salt of any one of embodiments 62-76, characterized by an X-ray powder diffraction (XRPD) pattern substantially as shown in FIG. 12.

Described herein as embodiment 78 is the salt of any one of embodiments 62-77, characterized by a differential scanning calorimetry (DSC) thermogram comprising an endotherm with an onset of about 150° C.±1° C.

Described herein as embodiment 79 is the salt of embodiment 78, characterized by a differential scanning calorimetry (DSC) thermograph substantially as shown in FIG. 13.

Described herein as embodiment 80 is the salt of any one of embodiments 62-79, characterized by a thermogravimetric analysis (TGA) thermogram comprising a weight loss of about 29.8% when heated from about 150° C. to about 250° C.

Described herein as embodiment 81 is the salt of embodiment 80, characterized by a thermogravimetric analysis (TGA) thermograph substantially as shown in FIG. 14.

Described herein as embodiment 82 is the salt of any one of embodiments 62-81, characterized by a dynamic vapor sorption (DVS) graph substantially as shown in FIG. 16.

Described herein as embodiment 83 is the salt of any one of embodiments 1-3, wherein the salt is a hydrobromide salt.

Described herein as embodiment 84 is the salt of embodiment 83, characterized by one or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 85 is the salt of embodiment 84, characterized by two or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 86 is the salt of embodiment 85, characterized by three or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 87 is the salt of embodiment 86, characterized by four or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 88 is the salt of embodiment 87, characterized by five or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 89 is the salt of embodiment 88, characterized by seven or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 90 is the salt of embodiment 89, characterized by ten or more X-ray powder diffraction (XRPD) peaks selected from: 4.9, 6.5, 7.0, 8.0, 9.2, 12.2, 17.9, 19.6, 20.3, 21.2 and 23.1, ±0.2° 2θ using X-ray wavelength of 1.54 Å, wherein the source of radiation is CuKα.

Described herein as embodiment 91 is the salt of embodiment 83, wherein the salt is a Compound A hydrobromide (“Form S-L”), characterized by an X-ray powder diffraction (XRPD) pattern comprising peaks at 7.0±0.2° 2θ using CuKα radiation.

Described herein as embodiment 92 is the salt of embodiment 83, wherein the salt is a Compound A hydrobromide (“Form S-L”), characterized by an X-ray powder diffraction (XRPD) pattern comprising peaks at 7.0 and 8.0±0.2° 2θ using CuKα radiation.

Described herein as embodiment 93 is the salt of embodiment 83, wherein the salt is a Compound A hydrobromide (“Form S-L”), characterized by an X-ray powder diffraction (XRPD) pattern comprising peaks at 7.0, 8.0, and 4.9±0.2° 2θ using CuKα radiation.

Described herein as embodiment 94 is the salt of embodiment 93, further characterized by a peak at 6.5±0.2° 2θ using CuKα radiation.

Described herein as embodiment 95 is the salt of embodiment 94, further characterized by a peak at 17.9±0.2° 2θ using CuKα radiation.

Described herein as embodiment 96 is the salt of any one of embodiments 83-95, characterized by an X-ray powder diffraction (XRPD) pattern substantially as shown in FIG. 17.

Described herein as embodiment 97 is the salt of any one of embodiments 83-96, characterized by a differential scanning calorimetry (DSC) thermograph substantially as shown in FIG. 18.

Described herein as embodiment 98 is the salt of any one of embodiments 1-3, wherein the salt is a hydrochloride salt.

Described herein as embodiment 99 is the salt of embodiment 98, characterized by one or more of the following data selected from the group consisting of:
(a) one or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα; and
(b) a thermogravimetric analysis (TGA) thermogram comprising a weight loss of about 6.81% when heated from about 150° C. to about 250° C.

Described herein as embodiment 100 is the salt of embodiment 99, characterized by one or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 101 is the salt of embodiment 100, characterized by two or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 102 is the salt of embodiment 101, characterized by three or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 103 is the salt of embodiment 102, characterized by four or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 104 is the salt of embodiment 103, characterized by five or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 105 is the salt of embodiment 104, characterized by seven or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 106 is the salt of embodiment 105, characterized by ten or more X-ray powder diffraction (XRPD) peaks selected from: 4.6, 6.6, 8.1, 12.1, 15.5, 18.1, 19.7, 20.5, 21.5, 27.0 and 27.8, ±0.2° 2θ using X-ray wavelength of 1.54 Å, wherein the source of radiation is CuKα.

Described herein as embodiment 107 is the salt of embodiment 99, wherein the salt is a Compound A hydrochloride (“Form S-T”), characterized by an X-ray powder diffraction (XRPD) pattern comprising peaks at 8.1±0.2° 2θ using CuKα radiation.

Described herein as embodiment 108 is the salt of embodiment 99, wherein the salt is a Compound A hydrochloride (“Form S-T”), characterized by an X-ray powder diffraction (XRPD) pattern comprising peaks at 8.1 and 20.5±0.2° 2θ using CuKα radiation.

Described herein as embodiment 109 is the salt of embodiment 99, wherein the salt is a Compound A hydrochloride (“Form S-T”), characterized by an X-ray powder diffraction (XRPD) pattern comprising peaks at 8.1, 20.5, and 6.6±0.2° 2θ using CuKα radiation.

Described herein as embodiment 110 is the salt of embodiment 109, further characterized by a peak at 19.7±0.2° 2θ using CuKα radiation.

Described herein as embodiment 111 is the salt of embodiment 110, further characterized by a peak at 21.5±0.2° 2θ using CuKα radiation.

Described herein as embodiment 112 is the salt of embodiment 111, further characterized by peaks at 18.1, 12.1, and 27.8±0.2° 2θ using CuKα radiation.

Described herein as embodiment 113 is the salt of any one of embodiments 98-112, characterized by an X-ray powder diffraction (XRPD) pattern substantially as shown in FIG. 20.

Described herein as embodiment 114 is the salt of any one of embodiments 98-113, characterized by a differential scanning calorimetry (DSC) thermograph substantially as shown in FIG. 21.

Described herein as embodiment 115 is the salt of any one of embodiments 98-114, characterized by a thermogravimetric analysis (TGA) thermogram comprising a weight loss of about 6.81% when heated from about 150° C. to about 250° C.

Described herein as embodiment 116 is the salt of embodiment 115, characterized by a thermogravimetric analysis (TGA) thermograph substantially as shown in FIG. 22.

Described herein as embodiment 117 is the salt of any one of embodiments 1-3, wherein the salt is an edisylate salt.

Described herein as embodiment 118 is the salt of embodiment 98, characterized by one or more of the following data selected from the group consisting of:
(a) one or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα; and
(b) a thermogravimetric analysis (TGA) thermogram comprising a weight loss of about 2.12% when heated from about 150° C. to about 250° C.

Described herein as embodiment 119 is the salt of embodiment 118, characterized by one or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 120 is the salt of embodiment 119, characterized by two or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 121 is the salt of embodiment 120, characterized by three or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 122 is the salt of embodiment 121, characterized by four or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 123 is the salt of embodiment 122, characterized by five or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 124 is the salt of embodiment 123, characterized by seven or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 125 is the salt of embodiment 124, characterized by ten or more X-ray powder diffraction (XRPD) peaks selected from: 6.3, 10.7, 11.3, 12.5, 13.3, 17.1, 17.5, 19.2, 19.5, 20.0 and 21.4, ±0.2° 2θ using X-ray wavelength of 1.54 Å, wherein the source of radiation is CuKα.

Described herein as embodiment 126 is the salt of embodiment 118, wherein the salt is a Compound A edisylate salt (“Form 1F”), characterized by an X-ray powder diffraction (XRPD) pattern comprising peaks at 6.3, 17.1, and 20.0±0.2° 2θ using CuKα radiation.

Described herein as embodiment 127 is the salt of embodiment 118, wherein the salt is a Compound A edisylate salt (“Form 1F”), characterized by an X-ray powder diffraction (XRPD) pattern comprising peaks at 6.3, 17.1, and 20.0±0.2° 2θ using CuKα radiation.

Described herein as embodiment 128 is the salt of embodiment 118, wherein the salt is a Compound A edisylate salt (“Form 1F”), characterized by an X-ray powder diffraction (XRPD) pattern comprising peaks at 6.3, 17.1, and 20.0±0.2° 2θ using CuKα radiation.

Described herein as embodiment 129 is the salt of embodiment 128, further characterized by a peak at 19.2±0.2° 2θ using CuKα radiation.

Described herein as embodiment 130 is the salt of embodiment 129, further characterized by a peak at 13.3±0.2° 2θ using CuKα radiation.

Described herein as embodiment 131 is the salt of embodiment 130, further characterized by peaks at 11.3, 10.7, and 12.5±0.2° 2θ using CuKα radiation.

Described herein as embodiment 132 is the salt of any one of embodiments 117-131, characterized by an X-ray powder diffraction (XRPD) pattern substantially as shown in FIG. 24.

Described herein as embodiment 133 is the salt of any one of embodiments 117-132, characterized by a differential scanning calorimetry (DSC) thermograph substantially as shown in FIG. 25.

Described herein as embodiment 134 is the salt of any one of embodiments 117-133, characterized by a thermogravimetric analysis (TGA) thermogram comprising a weight loss of about 2.12% when heated from about 150° C. to about 250° C.

Described herein as embodiment 135 is the salt of embodiment 134, characterized by a thermogravimetric analysis (TGA) thermograph substantially as shown in FIG. 26.

Described herein as embodiment 136 is the salt of any one of embodiments 1-3, wherein the salt is a hemiedisylate salt.

Described herein as embodiment 137 is the salt of embodiment 136, characterized by one or more of the following data selected from the group consisting of:
(a) one or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα; and
(b) a thermogravimetric analysis (TGA) thermogram comprising a weight loss of about 1.95% when heated from about 150° C. to about 300° C.

Described herein as embodiment 138 is the salt of embodiment 137, characterized by one or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 139 is the salt of embodiment 138, characterized by two or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 140 is the salt of embodiment 139, characterized by three or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 141 is the salt of embodiment 140, characterized by four or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 142 is the salt of embodiment 141, characterized by five or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 143 is the salt of embodiment 142, characterized by seven or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 144 is the salt of embodiment 143, characterized by ten or more X-ray powder diffraction (XRPD) peaks selected from: 5.0, 6.2, 6.7, 7.0, 9.5, 10.6, 12.9, 13.7, 14.3, 14.9, 17.0, 17.3, 18.4, 19.2, 19.7, 20.5 and 21.8, ±0.2° 2θ using X-ray wavelength of 1.54 Å, wherein the source of radiation is CuKα.

Described herein as embodiment 145 is the salt of embodiment 137, wherein the salt is a Compound A hemiedisylate salt (“Form 1G”), characterized by an X-ray powder diffraction (XRPD) pattern comprising peaks at 13.7, 6.7, and 20.5±0.2° 2θ using CuKα radiation.

Described herein as embodiment 146 is the salt of embodiment 137, wherein the salt is a Compound A hemiedisylate salt (“Form 1G”), characterized by an X-ray powder diffraction (XRPD) pattern comprising peaks at 13.7, 6.7, and 20.5±0.2° 2θ using CuKα radiation.

Described herein as embodiment 147 is the salt of embodiment 137, wherein the salt is a Compound A hemiedisylate salt (“Form 1G”), characterized by an X-ray powder diffraction (XRPD) pattern comprising peaks at 13.7, 6.7, and 20.5±0.2° 2θ using CuKα radiation.

Described herein as embodiment 148 is the salt of embodiment 147, further characterized by a peak at 17.3±0.2° 2θ using CuKα radiation.

Described herein as embodiment 149 is the salt of embodiment 148, further characterized by a peak at 14.9±0.2° 2θ using CuKα radiation.

Described herein as embodiment 150 is the salt of embodiment 149, further characterized by peaks at 14.3, 6.2, 21.8, 7.0, 5.0, 17.0, 12.9, 19.2, and 18.4±0.2° 2θ using CuKα radiation.

Described herein as embodiment 151 is the salt of any one of embodiments 136-150 characterized by an X-ray powder diffraction (XRPD) pattern substantially as shown in FIG. 27.

Described herein as embodiment 152 is the salt of any one of embodiments 136-151, characterized by a differential scanning calorimetry (DSC) thermograph substantially as shown in FIG. 28.

Described herein as embodiment 153 is the salt of any one of embodiments 136-152, characterized by a thermogravimetric analysis (TGA) thermogram comprising a weight loss of about 1.95% when heated from about 150° C. to about 300° C.

Described herein as embodiment 154 is the salt of embodiment 153, characterized by a thermogravimetric analysis (TGA) thermograph substantially as shown in FIG. 29.

Described herein as embodiment 155 is a co-crystal of Compound A

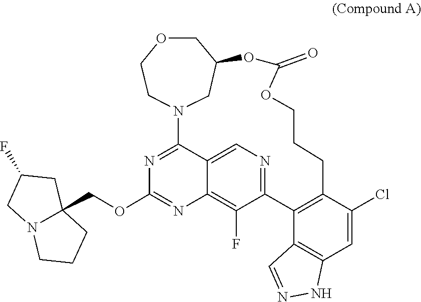 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

comprising Compound A and a coformer.

Described herein as embodiment 156 is the co-crystal of embodiment 155, wherein the coformer comprises quercetin, acetazolamide, L-tartaric acid, glycine, or glutaric acid.

Described herein as embodiment 157 is the co-crystal of embodiment 155 or 156, wherein the coformer comprises quercetin.

Described herein as embodiment 158 is the co-crystal of embodiment 157, characterized by one or more of the following data selected from the group consisting of:
(a) one or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα;
(b) a differential scanning calorimetry (DSC) thermogram comprising an endotherm with an onset of about 212° C.±1° C.; and
(c) a thermogravimetric analysis (TGA) thermogram comprising a weight loss when heated from above 160° C.

Described herein as embodiment 159 is the co-crystal of embodiment 158, characterized by one or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 160 is the co-crystal of embodiment 159, characterized by two or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 161 is the co-crystal of embodiment 160, characterized by three or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 162 is the co-crystal of embodiment 161, characterized by four or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 163 is the co-crystal of embodiment 162, characterized by five or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 164 is the co-crystal of embodiment 163, characterized by seven or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 165 is the co-crystal of embodiment 164, characterized by ten or more X-ray powder diffraction (XRPD) peaks selected from: 4.5, 4.9, 7.0, 9.5, 9.8, 11.8, 13.1, 15.4, 16.9, 19.1, 19.6 and 23.1, ±0.2° 2θ using X-ray wavelength of 1.54 Å, wherein the source of radiation is CuKα.

Described herein as embodiment 166 is the co-crystal of embodiment 158, wherein the co-crystal is a Compound A quercetin co-crystal (“Form C-P”), characterized by an X-ray powder diffraction (XRPD) pattern comprising peaks at 7.0±0.2° 2θ using CuKα radiation.

Described herein as embodiment 167 is the co-crystal of embodiment 158, wherein the co-crystal is a Compound A quercetin co-crystal (“Form C-P”), characterized by an X-ray powder diffraction (XRPD) pattern comprising peaks at 7.0 and 19.1±0.2° 2θ using CuKα radiation.

Described herein as embodiment 168 is the co-crystal of embodiment 158, wherein the co-crystal is a Compound A quercetin co-crystal (“Form C-P”), characterized by an X-ray powder diffraction (XRPD) pattern comprising peaks at 7.0, 19.1, and 9.5±0.2° 2θ using CuKα radiation.

Described herein as embodiment 169 is the co-crystal of embodiment 168, further characterized by a peak at 4.9±0.2° 2θ using CuKα radiation.

Described herein as embodiment 170 is the co-crystal of embodiment 169, further characterized by a peak at 11.8±0.2° 2θ using CuKα radiation.

Described herein as embodiment 171 is the co-crystal of embodiment 170, further characterized by peaks at 13.1, 16.9, 9.8, 19.6±0.2° 2θ using CuKα radiation.

Described herein as embodiment 172 is the co-crystal of any one of embodiments 157-171, characterized by an X-ray powder diffraction (XRPD) pattern substantially as shown in FIG. 31.

Described herein as embodiment 173 is the co-crystal of any one of embodiments 157-172, characterized by a differential scanning calorimetry (DSC) thermogram comprising an endotherm with an onset of about 212° C.±1° C.

Described herein as embodiment 174 is the co-crystal of embodiment 173, characterized by a differential scanning calorimetry (DSC) thermograph substantially as shown in FIG. 32.

Described herein as embodiment 175 is the co-crystal of any one of embodiments 157-174, characterized by a thermogravimetric analysis (TGA) thermogram comprising a weight loss when heated from above 160° C.

Described herein as embodiment 176 is the co-crystal of embodiment 175, characterized by a thermogravimetric analysis (TGA) thermograph substantially as shown in FIG. 33.

Described herein as embodiment 177 is the co-crystal of any one of embodiments 157-176, characterized by a dynamic vapor sorption (DVS) graph substantially as shown in FIG. 34.

Described herein as embodiment 178 is the co-crystal of embodiment 155 or 156, wherein the coformer comprises glycine hydrochloride.

Described herein as embodiment 179 is the co-crystal of embodiment 178, characterized by one or more of the following data selected from the group consisting of:
(a) one or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα;
(b) a differential scanning calorimetry (DSC) thermogram comprising an exotherm with an onset of about 200° C.±1° C.; and
(c) a thermogravimetric analysis (TGA) thermogram comprising a weight loss of about 24.04% when heated from about 200° C. to about 300° C.

Described herein as embodiment 180 is the co-crystal of embodiment 179, characterized by one or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 181 is the co-crystal of embodiment 180, characterized by two or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 182 is the co-crystal of embodiment 181, characterized by three or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 183 is the co-crystal of embodiment 182, characterized by four or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 184 is the co-crystal of embodiment 183, characterized by five or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 185 is the co-crystal of embodiment 184, characterized by seven or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 186 is the co-crystal of embodiment 185, characterized by ten or more X-ray powder diffraction (XRPD) peaks selected from: 4.7, 6.6, 8.1, 10.5, 12.0, 18.0, 19.7, 20.5, 21.5, 23.8 and 28.5, ±0.2° 2θ using X-ray wavelength of 1.54 Å, wherein the source of radiation is CuKα.

Described herein as embodiment 187 is the co-crystal of embodiment 179, wherein the co-crystal is a Compound A glycine hydrochloride co-crystal hydrate (“Form C1”), characterized by an X-ray powder diffraction (XRPD) pattern comprising peaks at 8.1±0.2° 2θ using CuKα radiation.

Described herein as embodiment 188 is the co-crystal of embodiment 179, wherein the co-crystal is a Compound A glycine hydrochloride co-crystal hydrate (“Form C1”), characterized by an X-ray powder diffraction (XRPD) pattern comprising peaks at 8.1 and 18.0±0.2° 2θ using CuKα radiation.

Described herein as embodiment 189 is the co-crystal of embodiment 179, wherein the co-crystal is a Compound A glycine hydrochloride co-crystal hydrate (“Form C1”), characterized by an X-ray powder diffraction (XRPD) pattern comprising peaks at 8.1, 18.0, and 23.8±0.2° 2θ using CuKα radiation.

Described herein as embodiment 190 is the co-crystal of embodiment 189, further characterized by a peak at 12.0±0.2° 2θ using CuKα radiation.

Described herein as embodiment 191 is the co-crystal of embodiment 190, further characterized by a peak at 20.5±0.2° 2θ using CuKα radiation.

Described herein as embodiment 192 is the co-crystal of embodiment 191, further characterized by peaks at 28.5, 19.7, and 6.6±0.2° 2θ using CuKα radiation.

Described herein as embodiment 193 is the co-crystal of any one of embodiments 178-192, characterized by an X-ray powder diffraction (XRPD) pattern substantially as shown in FIG. 35.

Described herein as embodiment 194 is the co-crystal of any one of embodiments 178-193, characterized by a differential scanning calorimetry (DSC) thermogram comprising an exotherm with an onset of about 200° C.±1° C.

Described herein as embodiment 195 is the co-crystal of embodiment 194, characterized by a differential scanning calorimetry (DSC) thermograph substantially as shown in FIG. 36.

Described herein as embodiment 196 is the co-crystal of any one of embodiments 178-195, characterized by a thermogravimetric analysis (TGA) thermogram comprising a weight loss of about 24.04% when heated from about 200° C. to about 300° C.

Described herein as embodiment 197 is the co-crystal of embodiment 196, characterized by a thermogravimetric analysis (TGA) thermograph substantially as shown in FIG. 37.

Described herein as embodiment 198 is the co-crystal of any one of embodiments 178-197, characterized by a dynamic vapor sorption (DVS) graph substantially as shown in FIG. 39.

Described herein as embodiment 199 is the co-crystal of embodiment 155 or 156, wherein the coformer comprises acetazolamide.

Described herein as embodiment 200 is the co-crystal of embodiment 199, characterized by one or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 201 is the co-crystal of embodiment 200, characterized by two or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 202 is the co-crystal of embodiment 201, characterized by three or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 203 is the co-crystal of embodiment 202, characterized by four or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 204 is the co-crystal of embodiment 203, characterized by five or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 205 is the co-crystal of embodiment 204, characterized by seven or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 206 is the co-crystal of embodiment 205, characterized by ten or more X-ray powder diffraction (XRPD) peaks selected from: 5.0, 6.3, 7.0, 17.0, 17.9, 18.7, 19.1, 19.7, 20.3, 21.4, 22.8, 23.2, 23.8 and 24.9, ±0.2° 2θ using X-ray wavelength of 1.54 Å, wherein the source of radiation is CuKα.

Described herein as embodiment 207 is the co-crystal of embodiment 199, wherein the co-crystal is a Compound A glycine hydrochloride co-crystal hydrate (“Form C1”), characterized by an X-ray powder diffraction (XRPD) pattern comprising peaks at 7.0±0.2° 2θ using CuKα radiation.

Described herein as embodiment 208 is the co-crystal of embodiment 199, wherein the co-crystal is a Compound A glycine hydrochloride co-crystal hydrate (“Form C1”), characterized by an X-ray powder diffraction (XRPD) pattern comprising peaks at 5.0 and 7.0±0.2° 2θ using CuKα radiation.

Described herein as embodiment 209 is the co-crystal of embodiment 199, wherein the co-crystal is a Compound A acetazolamide co-crystal (“Form C-E”), characterized by an X-ray powder diffraction (XRPD) pattern comprising peaks at 7.0, 5.0, and 23.2±0.2° 2θ using CuKα radiation.

Described herein as embodiment 210 is the co-crystal of embodiment 209, further characterized by a peak at 19.7±0.2° 2θ using CuKα radiation.

Described herein as embodiment 211 is the co-crystal of embodiment 210, further characterized by a peak at 17.9±0.2° 2θ using CuKα radiation.

Described herein as embodiment 212 is the co-crystal of embodiment 211, further characterized by peaks at 18.7, 17.0, 6.3, 19.1, 24.9, 20.3, 22.8, 23.8, and 21.4±0.2° 2θ using CuKα radiation.

Described herein as embodiment 213 is the co-crystal of any one of embodiments 198-212, characterized by an X-ray powder diffraction (XRPD) pattern substantially as shown in FIG. 40.

Described herein as embodiment 214 is the co-crystal of embodiment 155 or 156, wherein the coformer comprises tartaric acid.

Described herein as embodiment 215 is the co-crystal of embodiment 214, characterized by one or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 216 is the co-crystal of embodiment 215, characterized by two or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 217 is the co-crystal of embodiment 216, characterized by three or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 218 is the co-crystal of embodiment 217, characterized by four or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 219 is the co-crystal of embodiment 218, characterized by five or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 220 is the co-crystal of embodiment 219, characterized by seven or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 221 is the co-crystal of embodiment 220, characterized by nine or more X-ray powder diffraction (XRPD) peaks selected from: 14.1, 17.1, 17.7, 17.8, 19.3, 22.2, 24.6, 31.7 and 35.5, ±0.2° 2θ using X-ray wavelength of 1.54 Å, wherein the source of radiation is CuKα.

Described herein as embodiment 222 is the co-crystal of embodiment 214, wherein the co-crystal is a Compound A glycine hydrochloride co-crystal hydrate (“Form C1”), characterized by an X-ray powder diffraction (XRPD) pattern comprising peaks at 14.1±0.2° 2θ using CuKα radiation.

Described herein as embodiment 223 is the co-crystal of embodiment 214, wherein the co-crystal is a Compound A glycine hydrochloride co-crystal hydrate (“Form C1”), characterized by an X-ray powder diffraction (XRPD) pattern comprising peaks at 14.1 and 22.2±0.2° 2θ using CuKα radiation.

Described herein as embodiment 224 is the co-crystal of embodiment 214, wherein the co-crystal is a Compound A L-tartaric acid co-crystal (“Form C-D”), characterized by an X-ray powder diffraction (XRPD) pattern comprising peaks at 14.1, 22.2, and 17.1±0.2° 2θ using CuKα radiation.

Described herein as embodiment 225 is the co-crystal of embodiment 224, further characterized by a peak at 31.7±0.2° 2θ using CuKα radiation.

Described herein as embodiment 226 is the co-crystal of embodiment 225, further characterized by a peak at 24.6±0.2° 2θ using CuKα radiation.

Described herein as embodiment 227 is the co-crystal of embodiment 226, further characterized by peaks at 35.5, 17.7, and 19.3±0.2° 2θ using CuKα radiation.

Described herein as embodiment 228 is the co-crystal of any one of embodiments 214-227, characterized by an X-ray powder diffraction (XRPD) pattern substantially as shown in FIG. 41.

Described herein as embodiment 229 is the co-crystal of embodiment 155 or 156, wherein the coformer comprises glutaric acid.

Described herein as embodiment 230 is the co-crystal of embodiment 229, characterized by one or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 231 is the co-crystal of embodiment 230, characterized by two or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 232 is the co-crystal of embodiment 231, characterized by three or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 233 is the co-crystal of embodiment 232, characterized by four or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 234 is the co-crystal of embodiment 233, characterized by five or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 235 is the co-crystal of embodiment 234, characterized by seven or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 236 is the co-crystal of embodiment 235, characterized by nine or more X-ray powder diffraction (XRPD) peaks selected from: 6.3, 8.4, 11.2, 13.9, 14.2, 15.9, 16.8, 17.2, 17.5, 19.0, 24.3 and 24.6, ±0.2° 2θ using X-ray wavelength of 1.54 Å, wherein the source of radiation is CuKα.

Described herein as embodiment 237 is the co-crystal of embodiment 229, wherein the co-crystal is a Compound A glutaric acid co-crystal (“Form C-C”), characterized by an X-ray powder diffraction (XRPD) pattern comprising peaks at 6.3±0.2° 2θ using CuKα radiation.

Described herein as embodiment 238 is the co-crystal of embodiment 229, wherein the co-crystal is a Compound A glutaric acid co-crystal (“Form C-C”), characterized by an X-ray powder diffraction (XRPD) pattern comprising peaks at 6.3 and 16.8±0.2° 2θ using CuKα radiation.

Described herein as embodiment 239 is the co-crystal of embodiment 229, wherein the co-crystal is a Compound A glutaric acid co-crystal (“Form C-C”), characterized by an X-ray powder diffraction (XRPD) pattern comprising peaks at 6.3, 16.8, and 13.9±0.2° 2θ using CuKα radiation.

Described herein as embodiment 240 is the co-crystal of embodiment 239, further characterized by a peak at 19.0±0.2° 2θ using CuKα radiation.

Described herein as embodiment 241 is the co-crystal of embodiment 240, further characterized by a peak at 24.6±0.2° 2θ using CuKα radiation.

Described herein as embodiment 242 is the co-crystal of embodiment 241, further characterized by peaks at 14.2, 17.5, and 17.2±0.2° 2θ using CuKα radiation.

Described herein as embodiment 243 is the co-crystal of any one of embodiments 229-242 characterized by an X-ray powder diffraction (XRPD) pattern substantially as shown in FIG. 42.

Described herein as embodiment 244 is a crystalline form of Compound A

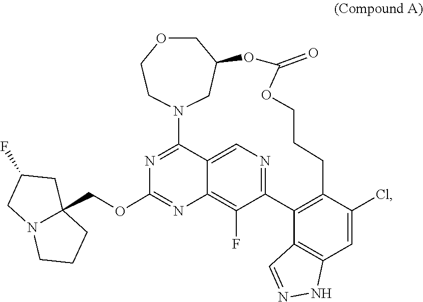 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the crystalline form is a free base hydrate.

Described herein as embodiment 244a is the crystalline form of Compound A, wherein the crystalline form is a free base hydrate (“Form 1”).

Described herein as embodiment 245 is the crystalline form of embodiment 244, characterized by one or more of the following data selected from the group consisting of:
(a) one or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα;
(b) one or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm;
(c) one or more ¹⁹F ssNMR spectrum peaks selected from: −170 and −137, ±0.2 ppm;
(d) a differential scanning calorimetry (DSC) thermogram comprising an exotherm with an onset of about 263° C.±1° C.; and
(e) a thermogravimetric analysis (TGA) thermogram comprising degradation upon melting above 263° C.

Described herein as embodiment 246 is the crystalline form of embodiment 245, characterized by one or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 247 is the crystalline form of embodiment 246, characterized by two or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 248 is the crystalline form of embodiment 247, characterized by three or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 249 is the crystalline form of embodiment 248, characterized by four or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 250 is the crystalline form of embodiment 249, characterized by five or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 251 is the crystalline form of embodiment 250, characterized by seven or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 252 is the crystalline form of embodiment 251, characterized by ten or more X-ray powder diffraction (XRPD) peaks selected from: 5.0, 7.0, 9.6, 11.9, 15.5, 17.0, 17.9, 19.1, 19.7, 23.2 and 24.9, ±0.2° 2θ using X-ray wavelength of 1.54 Å, wherein the source of radiation is CuKα.

Described herein as embodiment 253 is the crystalline form of embodiment 245, wherein the crystalline for is a Compound A free base hydrate (“Form 1”), characterized by an X-ray powder diffraction (XRPD) pattern comprising peaks at 7.0±0.2° 2θ using CuKα radiation.

Described herein as embodiment 254 is the crystalline form of embodiment 245, wherein the crystalline for is a Compound A free base hydrate (“Form 1”), characterized by an X-ray powder diffraction (XRPD) pattern comprising peaks at 7.0 and 5.0±0.2° 2θ using CuKα radiation.

Described herein as embodiment 255 is the crystalline form of embodiment 245, wherein the crystalline for is a Compound A free base hydrate (“Form 1”), characterized by an X-ray powder diffraction (XRPD) pattern comprising peaks at 7.0, 5.0, and 17.9±0.2° 2θ using CuKα radiation.

Described herein as embodiment 256 is the crystalline form of embodiment 255, further characterized by a peak at 17.0±0.2° 2θ using CuKα radiation.

Described herein as embodiment 257 is the crystalline form of embodiment 256, further characterized by a peak at 19.7±0.2° 2θ using CuKα radiation.

Described herein as embodiment 258 is the crystalline form of any one of embodiments 244-257, characterized by an X-ray powder diffraction (XRPD) pattern substantially as shown in FIG. 43.

Described herein as embodiment 259 is the crystalline for of any one of embodiments 244-258, characterized by differential scanning calorimetry (DSC) thermogram comprising an exotherm with an onset of about 263° C.±1° C.

Described herein as embodiment 260 is the crystalline form of embodiment 259, characterized by a differential scanning calorimetry (DSC) thermograph substantially as shown in FIG. 44.

Described herein as embodiment 261 is the crystalline form of any one of embodiments 244-260, characterized by a thermogravimetric analysis (TGA) thermogram comprising degradation upon melting above 263° C.

Described herein as embodiment 262 is the crystalline form of embodiment 261, characterized by a thermogravimetric analysis (TGA) thermograph substantially as shown in FIG. 45.

Described herein as embodiment 263 is the crystalline form of embodiment 245, characterized by one or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 264 is the crystalline form of embodiment 263, characterized by two or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 265 is the crystalline form of embodiment 264, characterized by three or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 266 is the crystalline form of embodiment 265, characterized by four or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 267 is the crystalline form of embodiment 266, characterized by five or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 268 is the crystalline form of embodiment 267, characterized by seven or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 269 is the crystalline form of embodiment 268, characterized by ten or more ¹³C ssNMR spectrum peaks selected from: 163.8, 161.4, 155.5, 142.1, 139.5, 135.8, 128.9, 122.9, 113.2, 110.2, 77.8, 73.2, 55.2 and 25.3, ±0.2 ppm.

Described herein as embodiment 270 is the crystalline form of any one of embodiments 244-269, characterized by a ¹³C ssNMR spectrum comprising a peak at 73.2±0.2 ppm.

Described herein as embodiment 271 is the crystalline form of any one of embodiments 244-269, characterized by a ¹³C ssNMR spectrum comprising a peak at 73.2 and 128.9±0.2 ppm.

Described herein as embodiment 272 is the crystalline form of any one of embodiments 244-269, characterized by a ¹³C ssNMR spectrum comprising a peak at 73.2, 128.9, and 142.1±0.2 ppm.

Described herein as embodiment 273 is the crystalline form of embodiment 272, further characterized by a peak at 139.5±0.2 ppm.

Described herein as embodiment 274 is the crystalline form of embodiment 273, further characterized by a peak at 110.2±0.2 ppm.

Described herein as embodiment 275 is the crystalline form of embodiment 274, further characterized by peaks at 122.6, 113.2, and 155.5±0.2 ppm.

Described herein as embodiment 276 is the crystalline form of embodiment 275, further characterized by peaks at 163.8 and 77.8±0.2 ppm.

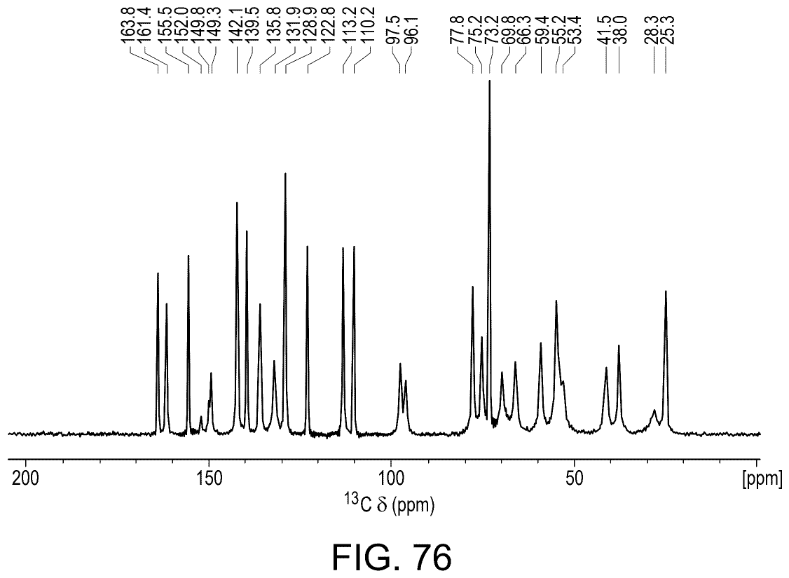

Described herein as embodiment 277 is the crystalline form of any one of embodiments 244-276, characterized by a ¹³C ssNMR spectrum substantially as shown in FIG. 76.

Described herein as embodiment 278 is the crystalline form of embodiment 245, characterized by one or more ¹⁹F ssNMR spectrum peaks selected from: one or more ¹⁹F ssNMR spectrum peaks selected from: −170 and −137, +0.2 ppm;

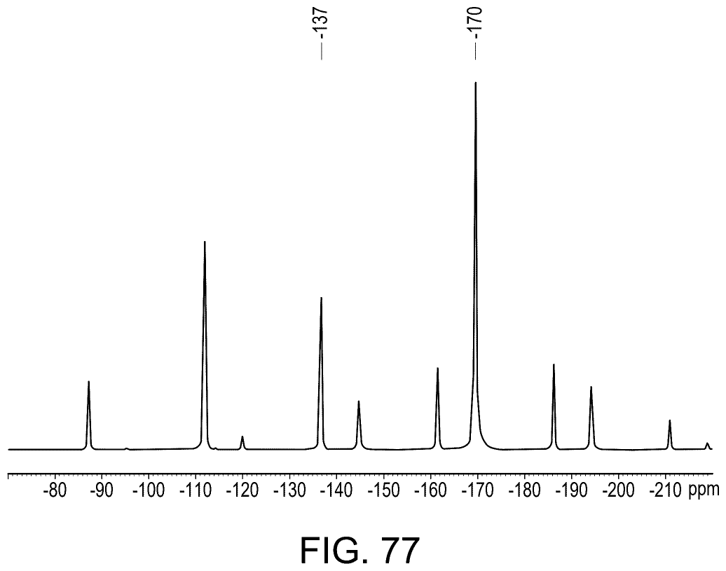

Described herein as embodiment 279 is the crystalline form of embodiment 278, characterized by a ¹⁹F spectrum substantially as showing in FIG. 77.

Described herein as embodiment 280 is the crystalline form of any one of embodiments 244-279, characterized by a ¹³C ssNMR spectrum comprising a peak at 73.2±0.2 ppm.

Described herein as embodiment 280a is the crystalline form of embodiment 244, wherein the crystalline form is a free base hydrate (“Form 2”).

Described herein as embodiment 281 is the crystalline form of embodiment 280a, characterized by one or more of the following data selected from the group consisting of:
(a) one or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα; and
(b) a thermogravimetric analysis (TGA) thermogram comprising a weight loss of about 5.55% when heated to about 150° C.

Described herein as embodiment 282 is the crystalline form of embodiment 281, characterized by one or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 283 is the crystalline form of embodiment 282, characterized by two or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 284 is the crystalline form of embodiment 283, characterized by three or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 285 is the crystalline form of embodiment 284, characterized by four or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 286 is the crystalline form of embodiment 285, characterized by five or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 287 is the crystalline form of embodiment 286, characterized by seven or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 288 is the crystalline form of embodiment 287, characterized by ten or more X-ray powder diffraction (XRPD) peaks selected from: 4.9, 7.0, 7.2, 15.6, 17.2, 18.1, 19.3, 19.9, 22.4, 23.4, 23.9, 25.1, 28.2, 30.0, 30.9 and 32.1, ±0.2° 2θ using X-ray wavelength of 1.54 Å, wherein the source of radiation is CuKα.

Described herein as embodiment 289 is the crystalline form of embodiment 281, wherein the crystalline form is a Compound A free base hydrate (“Form 2”), characterized by an X-ray powder diffraction (XRPD) pattern comprising peaks at 18.1±0.2° 2θ using CuKα radiation.

Described herein as embodiment 290 is the crystalline form of embodiment 281, wherein the crystalline form is a Compound A free base hydrate (“Form 2”), characterized by an X-ray powder diffraction (XRPD) pattern comprising peaks at 18.1 and 19.9±0.2° 2θ using CuKα radiation.

Described herein as embodiment 291 is the crystalline form of embodiment 281, wherein the crystalline form is a Compound A free base hydrate (“Form 2”), characterized by an X-ray powder diffraction (XRPD) pattern comprising peaks at 18.1, 19.9, and 7.0±0.2° 2θ using CuKα radiation.

Described herein as embodiment 292 is the crystalline form of embodiment 291, further characterized by a peak at 23.4±0.2° 2θ using CuKα radiation.

Described herein as embodiment 293 is the crystalline form of embodiment 292, further characterized by a peak at 19.3±0.2° 2θ using CuKα radiation.

Described herein as embodiment 294 is the crystalline form of embodiment 293, further characterized by peaks at 17.2, 25.1, 30.0, 7.2, 15.6, and 30.9±0.2° 2θ using CuKα radiation.

Described herein as embodiment 295 is the crystalline form of any one of embodiments 281-294, characterized by an X-ray powder diffraction (XRPD) pattern substantially as shown in FIG. 47.

Described herein as embodiment 296 is the crystalline form of any one of embodiments 281-295, characterized by a thermogravimetric analysis (TGA) thermogram comprising a weight loss of about 5.55% when heated to about 150° C.

Described herein as embodiment 297 is the crystalline form of embodiment 296, characterized by a thermogravimetric analysis (TGA) thermograph substantially as shown in FIG. 48.

Described herein as embodiment 298 is an amorphous form of Compound A

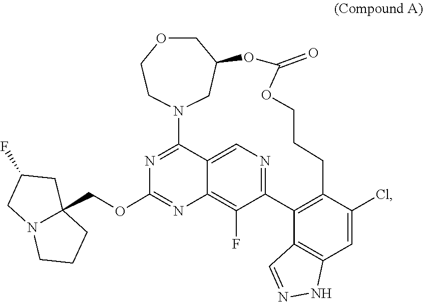 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous Compound A oxalate form (“Form A1”), characterized by a differential scanning calorimetry (DSC) thermograph substantially as shown in FIG. 49.

Described herein as embodiment 299 is the amorphous form of embodiment 298, characterized by a thermogravimetric analysis (TGA) thermograph substantially as shown in FIG. 50.

Described herein as embodiment 300 is the amorphous form of embodiment 298 or 299, characterized by an X-ray powder diffraction (XRPD) pattern substantially as shown in FIG. 52.

Described herein as embodiment 301 is an amorphous form of Compound A

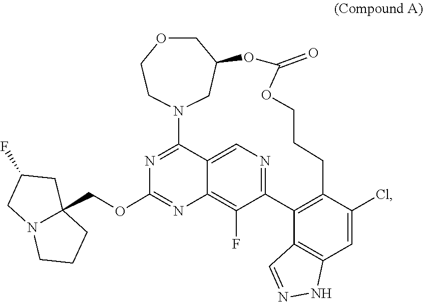 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous

Compound A tosylate form (“Form A1-A”), characterized by a differential scanning calorimetry (DSC) thermograph substantially as shown in FIG. 53.

Described herein as embodiment 302 is the amorphous form of embodiment 301, characterized by a thermogravimetric analysis (TGA) thermograph substantially as shown in FIG. 54.

Described herein as embodiment 303 is the amorphous form of embodiment 301 or 302, characterized by an X-ray powder diffraction (XRPD) pattern substantially as shown in FIG. 56.

Described herein as embodiment 304 is an amorphous form of Compound A

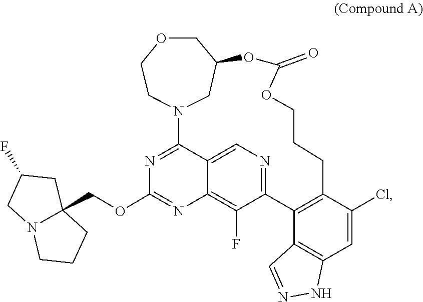 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous Compound A saccharinate form (“Form A1-B”), characterized by a differential scanning calorimetry (DSC) thermograph substantially as shown in FIG. 57.

Described herein as embodiment 305 is the amorphous form of embodiment 304, characterized by a thermogravimetric analysis (TGA) thermograph substantially as shown in FIG. 58.

Described herein as embodiment 306 is the amorphous form of embodiment 304 or 305, characterized by an X-ray powder diffraction (XRPD) pattern substantially as shown in FIG. 60.

Described herein as embodiment 307 is an amorphous form of Compound A

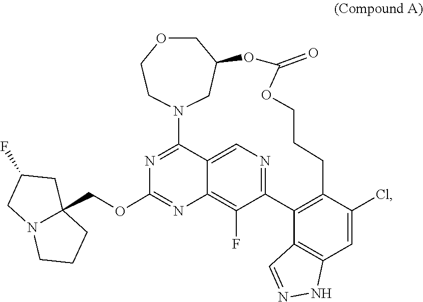 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous Compound A citrate form (“Form A1-C”), characterized by a differential scanning calorimetry (DSC) thermograph substantially as shown in FIG. 61.

Described herein as embodiment 308 is the amorphous form of embodiment 307, characterized by a thermogravimetric analysis (TGA) thermograph substantially as shown in FIG. 62.

Described herein as embodiment 309 is the amorphous form of embodiment 307 or 308, characterized by an X-ray powder diffraction (XRPD) pattern substantially as shown in FIG. 64.

Described herein as embodiment 310 is an amorphous form of Compound A

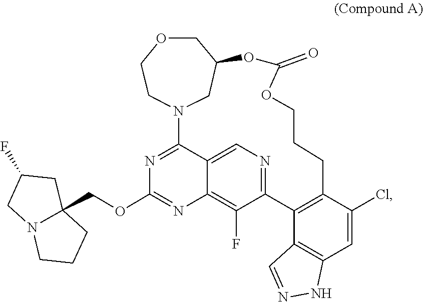 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous

Compound A free base form (“Form A2”), characterized by a differential scanning calorimetry (DSC) thermograph substantially as shown in FIG. 65.

Described herein as embodiment 311 is the amorphous form of embodiment 310, characterized by a thermogravimetric analysis (TGA) thermograph substantially as shown in FIG. 66.

Described herein as embodiment 312 is the amorphous form of embodiment 310 or 311, characterized by an X-ray powder diffraction (XRPD) pattern substantially as shown in FIG. 67.

Described herein as embodiment 313 is an amorphous form of Compound A

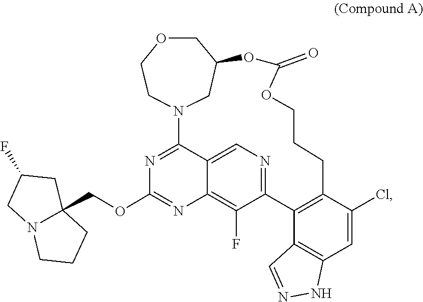 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous

Compound A besylate form (“Form A1-D”), characterized by a differential scanning calorimetry (DSC) thermograph substantially as shown in FIG. 68.

Described herein as embodiment 314 is the amorphous form of embodiment 313, characterized by a thermogravimetric analysis (TGA) thermograph substantially as shown in FIG. 69.

Described herein as embodiment 315 is the amorphous form of embodiment 313 or 314, characterized by an X-ray powder diffraction (XRPD) pattern substantially as shown in FIG. 71.

Described herein as embodiment 316 is an amorphous form of Compound A

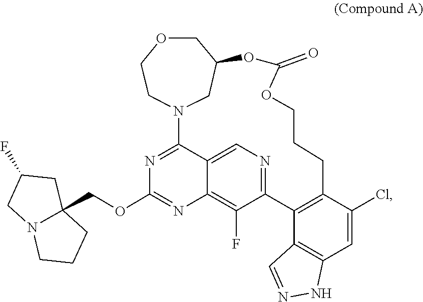 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]

wherein the amorphous form is an amorphous

Compound A esylate form (“Form A1-E”), characterized by a differential scanning calorimetry (DSC) thermograph substantially as shown in FIG. 72.

Described herein as embodiment 317 is the amorphous form of embodiment 316, characterized by a thermogravimetric analysis (TGA) thermograph substantially as shown in FIG. 73.

Described herein as embodiment 318 is the amorphous form of embodiment 316 or 317, characterized by an X-ray powder diffraction (XRPD) pattern substantially as shown in FIG. 75.

Described herein as embodiment 319 is a pharmaceutical composition comprising the salt, co-crystal, crystalline form, or amorphous form of any one of embodiments 1 to 318 and at least one pharmaceutically acceptable excipient.

Described herein as embodiment 320 is a method of treating a cancer in a subject in need thereof comprising administering to the subject a therapeutically effective amount of the salt, crystalline form, or amorphous form of any one of embodiments 1 to 318 or the pharmaceutical composition of embodiment 320.

Described herein as embodiment 321 is the method of embodiment 320, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

Described herein as embodiment 322 is the method according to embodiment 320 or 321, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

Described herein as embodiment 323 is the method according to embodiment 322, wherein the cancer is non-small cell lung cancer.

Described herein as embodiment 324 is the method according to embodiment 322, wherein the cancer is colorectal cancer.

Described herein as embodiment 325 is the method according to embodiment 322, wherein the cancer is pancreatic cancer.

Described herein as embodiment 326 is the method of embodiment 320 or 321, wherein the KRAS mutation is G12D.

Described herein as embodiment 327 is the method of embodiment 320 or 321, wherein the KRAS mutation is G12V.

Described herein as embodiment 328 is the method of embodiment 320 or 321, wherein the KRAS mutation is G12A.

Described herein as embodiment 329 is the e method of embodiment 320 or 321, wherein the KRAS mutation is G12S.

Described herein as embodiment 330 is the method of embodiment 320 or 321, wherein the KRAS mutation is G13D.

Described herein as embodiment 331 is the method of embodiment 320 or 321, wherein the KRAS mutation is G12C.

Described herein as embodiment 332 is the method of embodiment 320 or 321, wherein the KRAS mutation is Q61H.

Described herein as embodiment 333 is the method of embodiment 320 or 321, wherein the KRAS mutation is Q61L.

Described herein as embodiment 334 is the method of embodiment 320 or 321, wherein the cancer is hepatobiliary, ovarian, endometrial, pancreatic, colorectal, or non-small cell lung cancer.

Described herein as embodiment 335 is the method of embodiment 326, wherein the cancer is pancreatic cancer.

Described herein as embodiment 336 is the method of embodiment 327, wherein the pancreatic cancer is pancreatic ductal adenocarcinoma (PDAC).

Described herein as embodiment 337 is the method of embodiment 320 or 321, wherein the cancer is colorectal cancer.

Described herein as embodiment 338 is the method of embodiment 320 or 321, wherein the cancer is non-small cell lung cancer.

Described herein as embodiment 339 is the method of embodiment 330, wherein the non-small cell lung cancer is lung adenocarcinoma (LUAD).

Described herein as embodiment 340 is the method of embodiment 320 or 321, wherein the cancer is hepatobiliary cancer.

Described herein as embodiment 341 is the method of embodiment 320 or 321, wherein the cancer is ovarian cancer.

Described herein as embodiment 342 is the method of embodiment 320 or 321, wherein the cancer is endometrial cancer.

Described herein as embodiment 343 is the method of embodiment 320 or 321, wherein the cancer is colorectal cancer comprising a KRAS G12D mutation.

Described herein as embodiment 344 is the method of embodiment 320 or 321, wherein the cancer is colorectal cancer comprising a KRAS G12V mutation.

Described herein as embodiment 345 is the method of embodiment 320 or 321, wherein the cancer is colorectal cancer comprising a KRAS G13D mutation.

Described herein as embodiment 346 is the method of embodiment 320 or 321, wherein the cancer is colorectal cancer comprising a KRAS G12A mutation.

Described herein as embodiment 347 is the method of embodiment 320 or 321, wherein the cancer is colorectal cancer comprising a KRAS G12C mutation.

Described herein as embodiment 348 is the method of embodiment 320 or 321, wherein the cancer is colorectal cancer comprising a KRAS WT amplified mutation.

Described herein as embodiment 349 is the method of embodiment 320 or 321, wherein the cancer is pancreatic ductal adenocarcinoma (PDAC) comprising a KRAS G12D mutation.

Described herein as embodiment 350 is the method of embodiment 320 or 321, wherein the cancer is pancreatic ductal adenocarcinoma (PDAC) comprising a KRAS G12V mutation.

Described herein as embodiment 351 is the method of embodiment 320 or 321, wherein the cancer is pancreatic ductal adenocarcinoma (PDAC) comprising a KRAS G12C mutation.

Described herein as embodiment 352 is the method of embodiment 320 or 321, wherein the cancer is pancreatic ductal adenocarcinoma (PDAC) comprising a KRAS G12A mutation.

Described herein as embodiment 353 is the method of embodiment 320 or 321, wherein the cancer is pancreatic ductal adenocarcinoma (PDAC) comprising a KRAS G12R mutation.

Described herein as embodiment 354 is the method of embodiment 320 or 321, wherein the cancer is non-small cell lung cancer (NSCLC) comprising a KRAS G12C mutation.

Described herein as embodiment 355 is the method of embodiment 320 or 321, wherein the cancer is non-small cell lung cancer (NSCLC) comprising a KRAS G12D mutation.

Described herein as embodiment 356 is the method of embodiment 320 or 321, wherein the cancer is non-small cell lung cancer (NSCLC) comprising a KRAS G12V mutation.

Described herein as embodiment 357 is the method of embodiment 320 or 321, wherein the cancer is non-small cell lung cancer (NSCLC) comprising a KRAS G12A mutation.

Described herein as embodiment 358 is the method of embodiment 320 or 321, wherein the cancer is non-small cell lung cancer (NSCLC) comprising a KRAS WT amplified mutation.

Described herein as embodiment 359 is the method of embodiment 320 or 321, wherein the cancer is lung adenocarcinoma (LUAD) comprising a KRAS G12C mutation.

Described herein as embodiment 360 is the method of a embodiment 320 or 321, wherein the cancer is lung adenocarcinoma (LUAD) comprising a KRAS G12D mutation.

Described herein as embodiment 361 is the method of embodiment 320 or 321, wherein the cancer is lung adenocarcinoma (LUAD) comprising a KRAS G12V mutation.

Described herein as embodiment 362 is the method of embodiment 320 or 321, wherein the cancer is lung adenocarcinoma (LUAD) comprising a KRAS G12A mutation.

Described herein as embodiment 363 is the method of embodiment 320 or 321, wherein the cancer is lung adenocarcinoma (LUAD) comprising a KRAS WT amplified mutation.

Described herein as embodiment 364 is the method of embodiment 320 or 321, wherein the cancer is ovarian cancer comprising a KRAS G12C mutation.

Described herein as embodiment 365 is the method of embodiment 320 or 321, wherein the cancer is ovarian cancer comprising a KRAS G12D mutation.

Described herein as embodiment 366 is the method of embodiment 320 or 321, wherein the cancer is ovarian cancer comprising a KRAS G12V mutation.

Described herein as embodiment 367 is the method of embodiment 320 or 321, wherein the cancer is ovarian cancer comprising a KRAS G12A mutation.

Described herein as embodiment 368 is the method of embodiment 320 or 321, wherein the cancer is hepatobiliary cancer comprising a KRAS G12V mutation.

Described herein as embodiment 369 is the method of embodiment 320 or 321, wherein the cancer is hepatobiliary cancer comprising a KRAS G12C mutation.

Described herein as embodiment 370 is the method of embodiment 320 or 321, wherein the cancer is hepatobiliary cancer comprising a KRAS Q61H mutation.

Described herein as embodiment 371 is the method of embodiment 320 or 321, wherein the cancer is hepatobiliary cancer comprising a KRAS G12A mutation.

Described herein as embodiment 372 is the method of embodiment 320 or 321, wherein the cancer is hepatobiliary cancer comprising a KRAS G13D mutation.

Described herein as embodiment 373 is the method of embodiment 320 or 321, wherein the cancer is endometrial cancer comprising a KRAS G12D mutation.

Described herein as embodiment 374 is the method of embodiment 320 or 321, wherein the cancer is endometrial cancer comprising a KRAS G12V mutation.

Described herein as embodiment 375 is the method of embodiment 320 or 321, wherein the cancer is endometrial cancer comprising a KRAS G13D mutation.

Described herein as embodiment 376 is the method of embodiment 320 or 321, wherein the cancer is endometrial cancer comprising a KRAS G12A mutation.

Described herein as embodiment 377 is the method of embodiment 320 or 321, wherein the cancer is endometrial cancer comprising a KRAS G12C mutation.

Described herein as embodiment 378 is the method of embodiment 320 or 321, wherein the cancer is endometrial cancer comprising a KRAS WT amplified mutation.

Described herein as embodiment 379 is the method of any one of embodiments 320 to 378, wherein the subject is a human.

Described herein as embodiment 380 is a process for the preparation of a salt, wherein the salt is a malonate salt of Compound A and wherein the process comprises slurrying a free base of Compound A with malonic acid.

Described herein as embodiment 381 is the process of embodiment 380, wherein the process further comprises crystallizing the malonate salt of Compound A from acetonitrile.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

All patents and other publications identified are expressly incorporated herein by reference for the purpose of describing and disclosing, for example, the methodologies described in such publications that might be used in connection with the present invention. These publications are provided solely for their disclosure prior to the filing date of the present application.

### EXAMPLES

The following examples further illustrate the disclosed processes, but of course, should not be construed as in any way limiting their scope.

The following abbreviations are used herein: DSC refers to differential scanning calorimetry; TGA refers to thermogravimetric analysis; NMR refers to nuclear magnetic resonance; PXRD refers to powder X-ray diffraction; DVS refers to dynamic vapor sorption; FASSGF refers to fasted state simulated gastric fluid; FESSIF refers to fed state simulated intestinal fluid; PBS refers to phosphate-buffered saline; ACN refers to acetonitrile; DCM refers to dichloromethane; MeOH refers to methanol; and FB refers to free base.

### Materials and Methods

Commercially available reagents are used as is without further purification unless specified.

The crystalline forms disclosed herein may be characterized using conventional means, including physical constants, diffraction data, and spectral data. As shown in Table 1, twenty-one different forms were generated and characterized using DSC/TGA, PXRD and/or DVS, including 14 crystalline forms and 7 amorphous forms.

TABLE 1
Solid Forms of Compound A
Compound A - Forms

FB Hydrate (“Form 1”)
FB Hydrate (“Form 2”)
Maleate Salt Hydrate (“Form 1A”)
Citrate Salt Hydrate (“Form 1B”)
Compound A - Forms
Glycine HCl Co-crystal Hydrate (“Form C1”)
Malonate Salt Anhydrous (“Form 1E”)
Amorphous Oxalate (“Form A1”)
Amorphous Tosylate (“Form A1-A”)
Amorphous Saccharinate (“Form A1-B”)
Amorphous Citrate (“Form A1-C”)
Amorphous FB (“Form A2”)
HBr Salt Group (“Form S-L”)
HCl Salt Group (“Form S-T”)
Edisylate Salt (“Form 1F”)
Quercetin Co-crystal Group (“Form C-P”)
Acetazolamide Co-crystal Group (“Form C-E”)
Amorphous Besylate (“Form A1-D”)
Amorphous Esylate (“Form A1-E”)
Hemiedisylate Salt (“Form 1G”)
L-Tartaric Acid Co-crystal Group (“Form C-D”)
Glutaric Acid Co-crystal Group (“Form C-C”)

Crystalline forms include two hydrated free forms, five co-crystals of quercetin, glycine-HCl, acetazolamide, L-tartaric acid, glutaric acid and seven salts of malonate, maleate, HCl, HBr, edisylate, hemiedisylate, citrate, acetazolamide, and seven amorphous forms obtained from spray dried and amorphous dispersions in combination with citrate, saccharinate, tosylate, esylate, besylate and oxalate. Compound A malonate salt anhydrous Form 1E has the lowest hygroscopicity compared to all other forms.

### Differential Scanning Calorimetry (DSC)

Differential Scanning calorimetry-Method 1-Differential scanning calorimetry (DSC) measurements were performed on a TA instrument Q2000 or Discovery Series DSC using a crimped Tzero on normal crimped aluminum pan. Approximately 1-5 mg were scanned from 30-220° C. at 10° C./min under 50 mL/min of nitrogen flow. Data were analyzed with TRIOS v5.4 or TA Universal Analysis 1000 software version 4.5A. DSC data on the free base forms described herein was obtained utilizing Method 1.

Differential Scanning calorimetry-Method 2-Sample was analyzed using a DSC 2500 (TA Instruments Inc.) equipped with an RCS90 Discovery single-stage refrigeration system and auto sampler. The calibration of the instrument was made with an indium standard. Approximately 1.00 mg of the powder sample was weighed using a XP6 microbalance from Mettler Toledo (±0.002 mg) and placed on normal crimped aluminum pans. Samples were equilibrated at 25° C. prior to heating to 200° C. under a N₂ atmosphere (50 mL/min) at a rate of 2.5° C./min and temperature accuracy of 0.1° C. Sample was cooled until −20° C. at 20° C./min rate. Sample was reheated at 2.5° C./min until 350° C. Data were analyzed with TRIOS v5.4 or TA Universal Analysis 1000 software version 4.5A. DSC data on all salt and co-crystal forms described herein was obtained utilizing Method 2.

### Thermogravimetric Analysis (TGA)

Thermogravimetric Analysis (TGA)—TGA was performed on a TA Instruments Q5000 analyzer at 10° C./min from ambient to 300° C. Approximately 5 mg of sample was loaded on a platinum pan. The dry nitrogen flow was set at 25 mL/min. Data were analyzed with TRIOS v5.4 or TA Universal Analysis 1000 software version 4.5A.

### X-Ray Powder Diffraction (XRPD)

X-ray Powder Diffraction (XRPD)—Method 1-Powder X-ray diffractograms were collected using a PANalytical X'Pert PRO diffractometer. Samples were scanned at ambient temperature in continuous mode from 5-45° (20) with a step size of 0.0334° at 45 kV and 40 mA with CuKα radiation (1.54 Å). The incident beam path was equipped with a 0.02 rad roller slit, 15 mm mask, 4° fixed anti-scatter slit, and a programmable divergence slit. The diffracted beam was equipped with a 0.02 rad roller slit, programmable anti-scatter slit and a 0.02 mm nickel filter. Samples were prepared on a low background sample holder and placed on a spinning stage with a rotation time of 2 s.

XRPD—Method 2-Powder X-ray diffractograms were collected using a Bruker D8 Advance diffractometer equipped with a twin-twin optic and Eiger X-ray detector in reflection mode. The samples were scanned at ambient temperature in continuous mode from 3-40° 2θ with step size of 0.02° 2θ at 40 kV and 40 mA with CuKα radiation (1.54 Å). The incident beam path was equipped with primary soller slit 2.5-degree, secondary soller slit 4.0 and divergence slit 0.6 mm, slit mode is fix. Samples were prepared on a low background sample holder and placed on a spinning stage with a rotation time of 10 rev/min. Data were collected using DIFFRAC.MEASUREMENT CENTER (v. 7.5) and processed with DIFFRAC.EVA (v. 6.0).

### Moisture Sorption Analysis

Moisture Sorption Analysis-Moisture sorption isotherm was determined using a DVS Endeavour at 25° C. in an aluminum pan with water as the solvent (closed loop) and nitrogen gas flow at 200 sccm. The sample was analyzed from 40% to 0% to 90% to 0% to 90% to 0% to 40% RH in 5% RH steps. Each step was held until a dm/dt of less than 0.001%/min was achieved for 5 minutes (with a minimum of 10 minutes and maximum of 360 min/step).

### ¹H Solution NMR

¹H NMR spectra were collected using a Bruker Avance 400 MHz spectrometer. Each sample was scanned with an acquisition time of 4.7841 s, 16 transients, and a relaxation delay of 1 s at ambient temperature. Samples were prepared by dissolving 2-10 mg of material in 0.75 mL of DMSO-d₆. Spectra were processed and analyzed using ACD/Spectrus Processor 2019.1.2 software.

### ¹³C Solid-State NMR

All spectra were acquired at 11.7 T on a widebore Bruker Avance III spectrometer equipped with a 4 mm H/F/X magic angle spinning probe at 298 K. The field was set such that the adamantane ¹³C CH₂ resonance was at 38.48 ppm. Approximately 100 mg of sample was packed into a 4 mm zirconia rotor and a magic angle spinning frequency of 10 kHz was used.

¹³C spectra were acquired by ¹H-¹³C cross polarization with total suppression of sidebands sequence (CP-TOSS) using a 70-100% amplitude ramped ¹H pulse and a constant amplitude ¹³C pulse with maximum RF fields at ˜60 kHz and ˜50 kHz respectively. A CP contact time of 2 ms was used. 10 μs ¹³C pi pulses at 50 kHz were employed. During acquisition ¹H heteronuclear decoupling was achieved using SPINAL-64. 2916 transients were acquired for each spectrum using a recycle delay of 16.21 s. All other acquisition parameters can be found in the relevant data sets.

### ¹⁹F Solid-State NMR

All spectra were acquired at 14.1 T on a wide bore Bruker Avance III spectrometer equipped with a 4 mm H/F/X magic angle spinning probe at 298 K. ¹⁹F Chemical shifts were referenced externally using PTFE tape (polytetrafluoroethylene) with the ¹⁹F resonance set to −122 ppm. Approximately 100 mg of sample was packed into a 4 mm zirconia rotor and a magic angle spinning frequency of 10 or 14 kHz was used for the malonate salt and free base hydrate forms respectively.

Direct excitation ¹⁹F spectra were acquired with a 4.0 μs ¹⁹F excitation pulse. During acquisition ¹H heteronuclear decoupling was achieved using SPINAL-64. For the malonate salt form two separate experiments were acquired, each with 512 transients and a recycle delay of 32.76 s, the FIDs from the separate experiments were then co-added to give one spectrum consisting of 1024 transients. For the free base hydrate form, 128 transients were acquired in a single experiment with a recycle delay of 10.00 s All other acquisition parameters can be found in the relevant data sets. Data were processed and analyzed in TopSpin 3.6.4. Peak picking was done in TopSpin 3.6.4.

### Synthesis of Compound A

Compound A was prepared using the Intermediates and Syntheses depicted below:

### Scheme 1. Preparation of Intermediate B

### Intermediate B: Ethyl 4-(2-fluoro-8-(8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidin-7-yl)-6-(methoxymethoxy)naphthalen-1-yl)butanoate

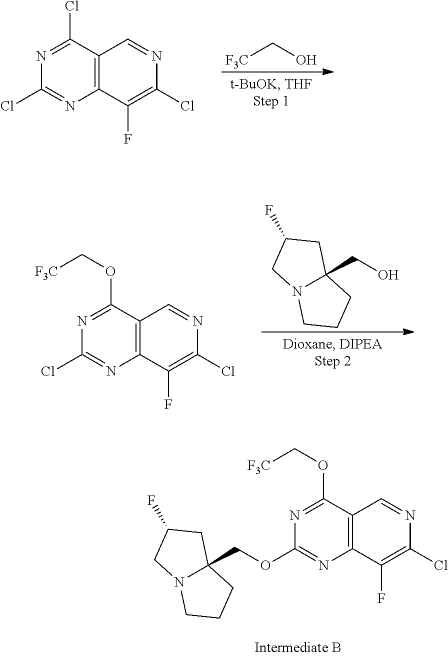 [SMILES: Fc1c(Cl)ncc2c(Cl)nc(Cl)nc12 ; OCC(F)(F)F ; Fc1c(Cl)ncc2c(OCC(F)(F)F)nc(Cl)nc12 ; OC[C@@]12CCCN1C[C@H](F)C2 ; Fc1c(Cl)ncc2c(OCC(F)(F)F)nc(OC[C@@]34CCCN3C[C@H](F)C4)nc12]

Step 1. 2,7-Dichloro-8-fluoro-4-(2,2,2-trifluoroethoxy)pyrido[4,3-d]pyrimidine. To a solution of 2,4,7-trichloro-8-fluoropyrido[4,3-d]pyrimidine (50.0 g, 198 mmol, Enamine) in tetrahydrofuran (750 mL) cooled to −60° C. was added 2,2,2-trifluoroethan-1-ol (18.82 g, 188 mmol), followed by t-BuOK (1 M in THF, 188 mL, 188 mmol) dropwise. The mixture was stirred at −60° C. for 2 h. The reaction mixture was quenched by addition of H₂O (1 L) at 20° C. and extracted with EtOAc. The combined organic layers were dried over Na₂SO₄, filtered, and concentrated. The residue was washed with petroleum ether (50 mL), then filtered. The filter cake was concentrated under reduced pressure to give 2,7-dichloro-8-fluoro-4-(2,2,2-trifluoroethoxy)pyrido[4,3-d]pyrimidine (50 g, 158 mmol, 84% yield) as yellow solid. ¹H NMR (400 MHz, CHLOROFORM-d) δ ppm 9.18 (s, 1H), 5.06-5.12 (m, 2H).

Step 2. 7-Chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine. To a solution of 2,7-dichloro-8-fluoro-4-(2,2,2-trifluoroethoxy)pyrido[4,3-d]pyrimidine (28 g, 89 mmol) in 1,4-dioxane (280 mL) was added (2R,7aS)-2-fluorotetrahydro-1H-pyrrolizin-7a(5H)-yl) methanol (16.93 g, 106 mmol) and DIPEA (46.4 mL, 266 mmol) in sequence. Then the mixture was stirred at 80° C. for 10 h. The reaction mixture was concentrated under reduced pressure and the residue was diluted with water and extracted with EtOAc. The combined organic layers were dried over Na₂SO₄, filtered, and concentrated. The residue was purified by column chromatography on silica gel, eluting with a gradient of 5-100% EtOAc in petroleum ether, to provide 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (Intermediate B) (28 g, 63.8 mmol, 72% yield) as yellow solid. m/z (ESI): 439.1/441.1 (M+H)⁺.

### Scheme 2: Preparation of Intermediate F

### Intermediate F: 5-(3-((tert-Butyldimethylsilyl)oxy)propyl)-6-chloro-1-(tetrahydro-2H-pyran-2-yl)-4-(4,4,5,5-tetramethyl-1,3,2-dioxaborolan-2-yl)-1H-indazole

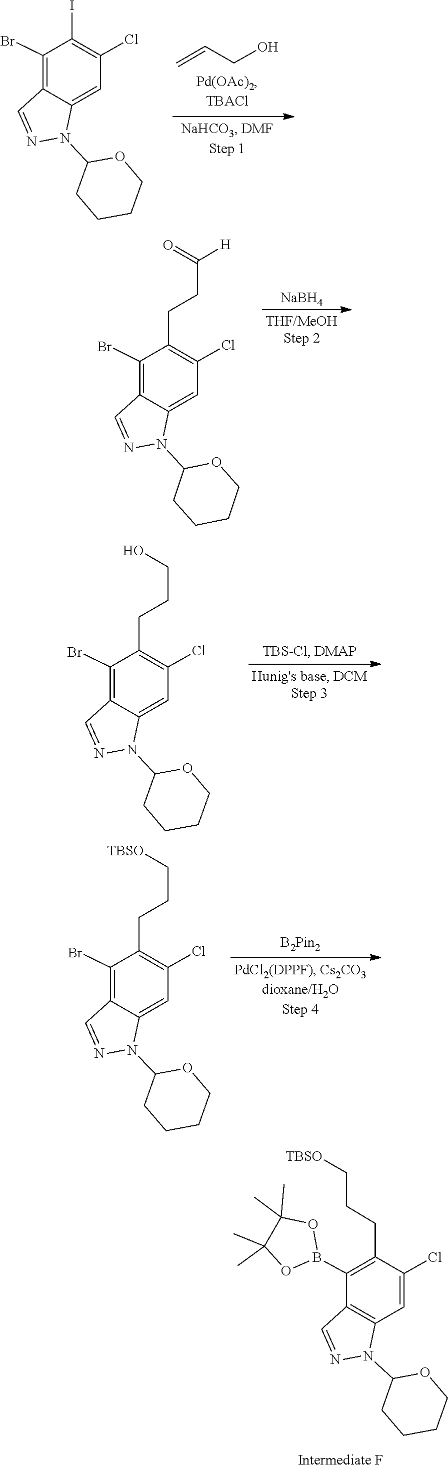

Step 1. 3-(4-Bromo-6-chloro-1-(tetrahydro-2H-pyran-2-yl)-1H-indazol-5-yl)propanal. To a 40 mL vial was charged with 4-bromo-6-chloro-5-iodo-1-(tetrahydro-2H-pyran-2-yl)-1H-indazole (3.00 g, 6.80 mmol, Lab Network), sodium bicarbonate (1.43 g, 17.0 mmol), TBACI (1.95 g, 6.82 mmol), and N,N-dimethylformamide (14 mL). The solution was degassed by nitrogen bubbling for 10 min. Then palladium (II) acetate (77 mg, 0.34 mmol) and allyl alcohol (0.7 mL, 10 mmol) were added at 50° C. The reaction mixture was stirred at 50° C. for 18 h. After cooling to rt, the reaction was diluted with saturated aqueous ammonium chloride and extracted with EtOAc. The organic layer was washed with saturated aqueous sodium chloride, dried over sodium sulfate, and concentrated. The crude material was purified by column chromatography on silica gel, eluting with a gradient of 0-50% 3:1 EtOAc/EtOH in heptane, to provide 3-(4-bromo-6-chloro-1-(tetrahydro-2H-pyran-2-yl)-1H-indazol-5-yl) propanal (2.12 g, 5.70 mmol, 84% yield) as light-orange oil. m/z (ESI): 371.0 (M+H)⁺.

Step 2: 3-(4-Bromo-6-chloro-1-(tetrahydro-2H-pyran-2-yl)-1H-indazol-5-yl) propan-1-ol. To a 100-mL round-bottom flask was added 3-(4-bromo-6-chloro-1-(tetrahydro-2H-pyran-2-yl)-1H-indazol-5-yl) propanal (1.06 g, 2.86 mmol) in tetrahydrofuran (5 mL)/methanol (5 mL). The reaction mixture was cooled to 0° C. Then, sodium borohydride (0.11 g, 2.86 mmol) was slowly added in portion. The reaction mixture was stirred at 0° C. for 30 min, then was slowly quenched with saturated NH₄Cl and extracted with EtOAc. The combined organic layers were washed with brine, dried over MgSO₄, filtered, and concentrated under reduced pressure. 3-(4-Bromo-6-chloro-1-(tetrahydro-2H-pyran-2-yl)-1H-indazol-5-yl) propan-1-ol (1.03 g, 2.76 mmol, 97% yield) was obtained as light-yellow oil without further purification. m/z (ESI): 289.0 (M-THP+H)⁺.

Step 3: 4-Bromo-5-(3-((tert-butyldimethylsilyl)oxy)propyl)-6-chloro-1-(tetrahydro-2H-pyran-2-yl)-1H-indazole. To a stirred solution of 3-(4-bromo-6-chloro-1-(tetrahydro-2H-pyran-2-yl)-1H-indazol-5-yl) propan-1-ol (1.03 g, 2.76 mmol) and 1,1′-dimethyltriethylamine (0.53 mL, 3.0 mmol) in dichloromethane (10 mL) in a 40 mL vial was added tert-butyldimethylsilyl chloride (0.46 g, 3.03 mmol) and 4-(dimethylamino)pyridine (34 mg, 0.28 mmol) at 0° C. After stirring at 0° C. for 2 h, the crude material was purified by column chromatography on silica gel, eluting with a gradient of 0-30% 3:1 EtOAc/EtOH in heptane, to provide 4-bromo-5-(3-((tert-butyldimethylsilyl)oxy)propyl)-6-chloro-1-(tetrahydro-2H-pyran-2-yl)-1H-indazole (1.12 g, 2.3 mmol, 83% yield) as colorless oil. m/z (ESI): 487.1 (M+H)⁺.

Step 4. 5-(3-((tert-Butyldimethylsilyl)oxy)propyl)-6-chloro-1-(tetrahydro-2H-pyran-2-yl)-4-(4,4,5,5-tetramethyl-1,3,2-dioxaborolan-2-yl)-1H-indazole. To a solution of 4-bromo-5-(3-((tert-butyldimethylsilyl)oxy)propyl)-6-chloro-1-(tetrahydro-2H-pyran-2-yl)-1H-indazole (3.4 g, 7.0 mmol), 4,4,4′,4′,5,5,5′,5′-octamethyl-2,2′-bi (1,3,2-dioxaborolane) (8.85 g, 34.8 mmol) in 1,4-dioxane (80 mL) and water (10 mL) was added Pd(dppf)Cl₂ (0.51 g, 0.7 mmol) and Cs₂CO₃ (6.81 g, 20.9 mmol) under N₂. The reaction mixture was heated at 120° C. for 5 h. After cooling to rt, the reaction mixture was diluted with water and extracted with EtOAc. The combined organic layers were washed with brine, dried over Na₂SO₄, filtered, and concentrated. The residue was purified by column chromatography on silica gel, eluting with a gradient of 10-100% EtOAc in petroleum ether, to give 5-(3-((tert-butyldimethylsilyl)oxy)propyl)-6-chloro-1-(tetrahydro-2H-pyran-2-yl)-4-(4,4,5,5-tetramethyl-1,3,2-dioxaborolan-2-yl)-1H-indazole (Intermediate F) (3.3 g, 6.2 mmol, 88% yield) as yellow oil. m/z (ESI): 535.3/537.2 (M+H)⁺.

### Scheme 3. Synthesis of Compound A

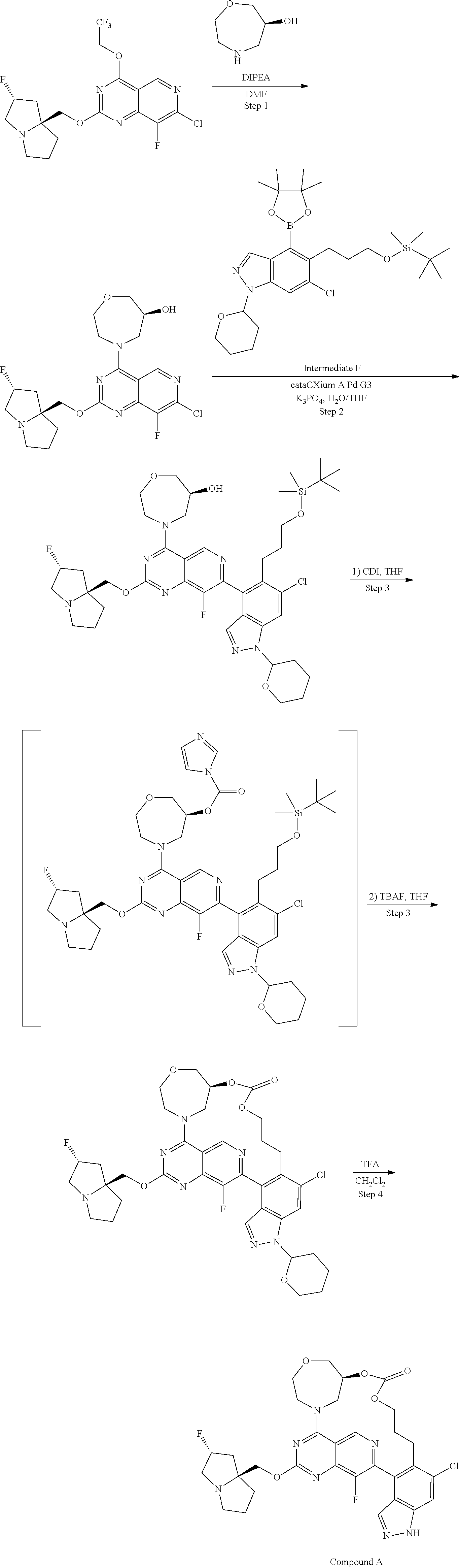

Step 1. (S)-4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol. A 40-mL vial was charged with DIPEA (3.2 mL, 18.2 mmol), (S)-[1,4]oxazepan-6-ol (1.1 mL, 9.12 mmol, J&W Pharmlab), 7-chloro-8-fluoro-2-(((2R,7aS)-2-fluorotetrahydro-1H-pyrrolizin-7a(5H)-yl)methoxy)-4-(2,2,2-trifluoroethoxy)pyrido[4,3-d]pyrimidine (2.00 g, 4.56 mmol, Step 2 in Intermediate B) and N,N-dimethylformamide (20 mL). The reaction mixture was stirred at rt for 1 h. Water and DCM were added. The organic layer was separated, dried over Na₂SO₄, filtered, and concentrated. The residue was purified by column chromatography on silica gel, eluting with 0-85% 3:1 EtOAc/EtOH (with 2% triethylamine) in heptane, to yield(S)-4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (1.40 g, 3.07 mmol, 67% yield). m/z (ESI): 456.0 (M+H)⁺.

Step 2. (6S)-4-(7-(5-(3-((tert-Butyldimethylsilyl)oxy)propyl)-6-chloro-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol. A 40 mL vial was charged with(S)-4-(7-chloro-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (0.40 g, 0.88 mmol), 5-(3-((tert-butyldimethylsilyl)oxy)propyl)-6-chloro-1-(tetrahydro-2H-pyran-2-yl)-4-(4,4,5,5-tetramethyl-1,3,2-dioxaborolan-2-yl)-1H-indazole (0.70 g, 1.32 mmol) (Intermediate F), potassium phosphate (0.75 g, 3.51 mmol), cataCXium A Pd G3 (0.13 g, 0.18 mmol). The vial was purged with nitrogen gas and then the reactants were suspended in degassed tetrahydrofuran (7.5 mL) and water (1.5 mL). The vial was then sealed, and the reaction mixture was heated to 80° C. for 2 h. After cooling to rt, the crude mixture was purified by column chromatography on silica gel, eluting with a gradient of 0-85% 3:1 EtOAc/EtOH (with 2% triethylamine) in heptane, to yield (6S)-4-(7-(5-(3-((tert-butyldimethylsilyl)oxy)propyl)-6-chloro-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (0.49 g, 0.59 mmol, 68% yield). m/z (ESI): 828.2 (M+H)⁺.

Step 3. (26S)-18-Chloro-32-fluoro-4-(((2R,7aS)-2-fluorotetrahydro-1H-pyrrolo[1,2-a]pyrrol-7a(5H)-yl)methoxy)-N-(tetrahydro-2H-pyran-2-yl)-23,25,28-trioxa-1,3,5,9,14,15-hexaazahexacyclo[24.4.1.1˜6,10˜.0˜2,7˜.0˜11,19˜.0˜12,16˜]-dotriaconta-2,4,6,8,10(32),11,13,16,18-nonaen-24-one. A 40 mL vial was charged with (6S)-4-(7-(5-(3-((tert-butyldimethylsilyl)oxy)propyl)-6-chloro-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-ol (0.49 g, 0.59 mmol) and 1,1′-carbonyldiimidazole (0.24 g, 1.49 mmol) and tetrahydrofuran (6 mL). The reaction mixture was allowed to stir at 40° C. until complete transformation to (6S)-4-(7-(5-(3-((tert-butyldimethylsilyl)oxy)propyl)-6-chloro-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)pyrido[4,3-d]pyrimidin-4-yl)-1,4-oxazepan-6-yl 1H-imidazole-1-carboxylate was observed. m/z (ESI): 922.8 (M+H)⁺.

The mixture was then diluted with THF (15 mL) and tetrabutylammonium fluoride (1.0 M in THF, 1.5 mL, 1.5 mmol) was added and the reaction mixture was allowed to stir at 40° C. until completion. The reaction was then concentrated under reduced pressure and the crude oil that was purified by column chromatography on silica gel, eluting with a gradient of 0-85% of a 3:1 EtOAc:EtOH (with 2% triethylamine) in heptane, to yield (26S)-18-chloro-32-fluoro-4-(((2R,7aS)-2-fluorotetrahydro-1H-pyrrolo[1,2-a]pyrrol-7a(5H)-yl)methoxy)-N-(tetrahydro-2H-pyran-2-yl)-23,25,28-trioxa-1,3,5,9,14,15-hexaazahexacyclo[24.4.1.1˜6,10˜.0˜2,7˜.0˜11,19˜.0˜12,16˜]-dotriaconta-2,4,6,8,10(32),11,13,16,18-nonaen-24-one (0.32 g, 0.43 mmol, 72% yield). m/z (ESI): 740.8 (M+H)⁺.

Step 4. (26S)-18-chloro-32-fluoro-4-(((2R,7aS)-2-fluorotetrahydro-1H-pyrrolo[1,2-a]pyrrol-7a(5H)-yl)methoxy)-23,25,28-trioxa-1,3,5,9,14,15-hexaazahexacyclo[24.4.1.1˜6,10˜.0˜2,7˜.0˜11,19˜.0˜12,16˜]dotriaconta-2,4,6,8,10(32),11,13,16,18-nonaen-24-one. (26S)-18-Chloro-32-fluoro-4-(((2R,7aS)-2-fluorotetrahydro-1H-pyrrolo[1,2-a]pyrrol-7a(5H)-yl)methoxy)-N-(tetrahydro-2H-pyran-2-yl)-23,25,28-trioxa-1,3,5,9,14,15-hexaazahexacyclo[24.4.1.1˜6, 10˜.0˜2,7˜.0˜11, 19˜.0˜12,16˜]-dotriaconta-2,4,6,8,10(32),11,13,16,18-nonaen-24-one (0.32 g, 0.43 mmol) dissolved in DCM (2.1 mL) was treated with trifluoroacetic acid (2.1 mL) and the reaction mixture was stirred at rt for 1 h. The volatiles were removed under reduced pressure and the crude product was purified by reverse phase chromatography to yield (26S)-18-chloro-32-fluoro-4-(((2R,7aS)-2-fluorotetrahydro-1H-pyrrolo[1,2-a]pyrrol-7a(5H)-yl)methoxy)-23,25,28-trioxa-1,3,5,9,14,15-hexaazahexacyclo[24.4.1.1˜6,10˜.0˜2,7˜.0˜11,19˜.0˜12,16˜]dotriaconta-2,4,6,8,10 (32),11,13,16,18-nonaen-24-one bis(2,2,2-trifluoroacetate) (Compound A) (0.14 g, 0.16 mmol, 36% yield). m/z (ESI): 655.9 (M+H)⁺. ¹H NMR (400 MHz, METHANOL-d₄) δ ppm 9.37-9.54 (m, 1H), 7.82-7.89 (m, 1H), 7.78 (s, 1H), 5.47-5.70 (m, 2H), 4.91-4.98 (m, 1H), 4.80-4.85 (m, 1H), 4.72 (s, 2H), 4.22-4.34 (m, 1H), 3.84-4.16 (m, 7H), 3.74-3.83 (m, 1H), 3.55-3.67 (m, 1H), 3.41-3.55 (m, 2H), 2.96-3.07 (m, 1H), 2.55-2.84 (m, 3H), 2.29-2.49 (m, 3H), 2.12-2.27 (m, 1H), 1.80-1.93 (m, 2H). ¹⁹F NMR (376 MHz, METHANOL-d₄) δ ppm −77.32 (TFA), −141.67 (s), −174.08 (s).

### Crystallization Conditions

The following Illustrative conditions were used to obtain Compound A Form 1, which was then used to obtain the other disclosed forms, as described herein, except for Form 1B, which was obtained from Form A1-C.

Form 1 was prepared by crystallizing Compound A from a solution of dichloromethane and trifluoroacetic acid at a temperature of 20° C. over a period of 2 hours.

Form 1A was prepared by slurrying Form 1 and maleic acid in a mixture of dichloromethane and methanol at a temperature of 25° C. for 24 hours.

Form 1E was prepared by slurrying Form 1 and malonic acid in acetonitrile at a temperature of 25° C. for 24 hours. Form 1E was also prepared by adding enough Compound A free base and malonic acid as a counterion solid in acetonitrile to form a mixture having excess solids. The mixture was then agitated in a sealed vial at ambient temperature (˜23-25° C.) for 24 h, after which period the solids were isolated by centrifuge filtration.

Form A1 was prepared by slurrying Form 1 and oxalic acid in a mixture of acetonitrile and methanol at a temperature of 60° C. for 48 hours.

Form C1 was prepared by slurrying Form 1 and glycine hydrochloride in a mixture of isopropanol:water (90:10) at a temperature of 25° C. for 3 days.

Form A1-A was prepared by slurrying Form 1 and toluene sulfonic acid in a mixture of acetonitrile and methanol at a temperature of 25° C. for 24 hours.

Form A1-B was prepared by slurrying Form 1 and saccharin in a mixture of dichloromethane and methanol at a temperature of 25° C. for 24 hours.

Form A2 was prepared from spray drying a solution of Form 1 (10 mg/ml in 50:50 dichloromethane:methanol).

Form 2 was prepared by slurrying Form 1 in water at a temperature of 25° C. for 24 hours.

Form S-L was prepared by slurrying Form 1 and hydrobromic acid in a mixture of acetonitrile:methanol (50:50% v/v) at a temperature of 60° C. for 48 hours.

Form A1-C was prepared by slurrying Form 1 and citric acid in acetonitrile at a temperature of 25° C. for 24 hours.

Form S-T was prepared by slurrying Form 1 and hydrochloric acid in methanol at a temperature of 60° C. for 3 days.

Form C-P was prepared by slurrying Form 1 and quercetin in acetonitrile at a temperature of 25° C. for 24 hours.

Form 1F was prepared by slurrying Form 1 and 1,2-ethanedisulfonic acid in methanol at a temperature of 60° C. for 2 hours.

Form C-E was prepared by slurrying Form 1 and acetazolamide in ethanol at a temperature of 25° C. for 24 hours.

Form A1-E was prepared by slurrying a 1:1.2 mixture of Form 1 and ethane sulfonic acid in acetonitrile at a temperature of 60° C. for 24 hours.

Form A1-D was prepared by slurrying a mixture of Form 1 and benzene sulfonic acid in acetonitrile at a temperature of 60° C. for 2 hours.

Form 1G was prepared by slurrying a 1:0.5 mixture of Form 1 and ethane sulfonic acid in methanol at a temperature of 60° C. for 48 hours.

Form C-D was prepared by slurrying a mixture of Form 1 and tartaric acid in an acetonitrile slurry at a temperature of 25° C. for 24 hours.

Form 1B was prepared by slurrying Form A1-C in acetone at a temperature of 25° C. for 24 hours.

Form C-C was prepared by slurrying a mixture of Form 1 and glutaric acid in an ethanol slurry at a temperature of 25° C. for 24 hours.

### Single Crystal Studies

The crystal structure of Compound A was determined by single crystal X-ray diffraction at room temperature and identified this material as an anhydrous form. Experimental PXRD confirmed structure solution was accurate within reason and representative of the bulk material. There was significant void volume (˜11%) to accommodate additional solvents. A stereochemistry was assigned to the molecule (Z′=1) in the asymmetric unit based on the Flack parameter. The chiral atoms in this structure are: C11(S), C25(S), C27(R).

TABLE 2

| Crystal Description | Clear colorless to light yellow needles | |
|---|---|---|
| Crystal Size (mm) | 0.26 × 0.05 × 0.03 | |
| Space Group | I41 | (80) |
| Cell Parameters (Å) | 25.15130(17), 25.15130(17), 10.01388(12) | |
| Cell Angles (°) | 90, 90, 90 | |
| Volume (Å³) | 6334.66 | (11) |
| Z′/Z | 1/8 | |
| Calc. Density (g/cm³) | 1.376 | |
| R-factor (%) | 6.01 | |
| Temp (K) | 100 | (1) |
| Flack | 0.025 | (13) |

### Compound A Malonate Salt Anhydrous (“Form 1E”)

The DSC thermograph of Form 1E is shown in FIG. 3 showing the absence of a thermal event below 150° C. indicating an anhydrous form. Form 1E did not exhibit a melting point. Both the counter ion and Compound A Form 1E start to degrade above about 170° C. as observed in the DSC (endothermic event) (FIG. 4).

The TGA thermograph of Form 1E is shown in FIG. 4 showing no weight loss below 150° C. Form 1E showed degradation starting at 170° C. with a 13.4% weight loss in TGA (13.7% wt. theoretical). corresponding to 1 equivalent of malonic acid. The additional step weight loss in the TGA corresponds to the degradation of Compound A.

The XRPD of Form 1E is shown in FIG. 2 having the peaks listed in Table 3.

TABLE 3
XRPD peaks of Form 1E

| Pos. [°2θ] | d-spacing [Å] | Rel. Int. [%] |
|---|---|---|
| 7.0 | 12.69 | 20.94 |
| 9.3 | 9.51 | 19.44 |
| 9.8 | 8.99 | 7.37 |
| 11.0 | 8.06 | 20.16 |
| 11.6 | 7.62 | 24.32 |
| 14.7 | 6.01 | 14.55 |
| 15.2 | 5.83 | 38.05 |
| 15.6 | 5.70 | 19.79 |
| 16.7 | 5.30 | 100 |
| 17.7 | 5.00 | 46.33 |
| 18.6 | 4.77 | 59.58 |
| 19.4 | 4.57 | 59.18 |
| 20.6 | 4.31 | 27.77 |
| 22.0 | 4.04 | 22.52 |
| 22.9 | 3.89 | 65.84 |
| 23.3 | 3.82 | 35.89 |
| 23.9 | 3.73 | 10.14 |
| 24.6 | 3.61 | 41.43 |
| 25.9 | 3.44 | 7.97 |
| 26.2 | 3.40 | 15.39 |
| 26.8 | 3.33 | 12.35 |
| 27.1 | 3.29 | 14.25 |
| 28.0 | 3.18 | 31.72 |
| 28.8 | 3.10 | 5.89 |
| 29.5 | 3.03 | 28.99 |
| 30.2 | 2.96 | 9.33 |
| 30.6 | 2.92 | 5.28 |
| 31.7 | 2.83 | 22.81 |
| 33.3 | 2.69 | 7.89 |
| 33.7 | 2.66 | 7.77 |
| 34.2 | 2.62 | 6.06 |
| 35.9 | 2.50 | 6.83 |
| 36.6 | 2.45 | 4.55 |
| 38.0 | 2.37 | 5.23 |
| 39.1 | 2.30 | 2.50 |
| 40.4 | 2.23 | 3.91 |
| 41.2 | 2.19 | 5.06 |
| 43.1 | 2.10 | 2.28 |

The ¹H NMR spectrum of Form 1E is shown in FIG. 5 and shows that the Compound A:malonic acid stoichiometry was 1:1.

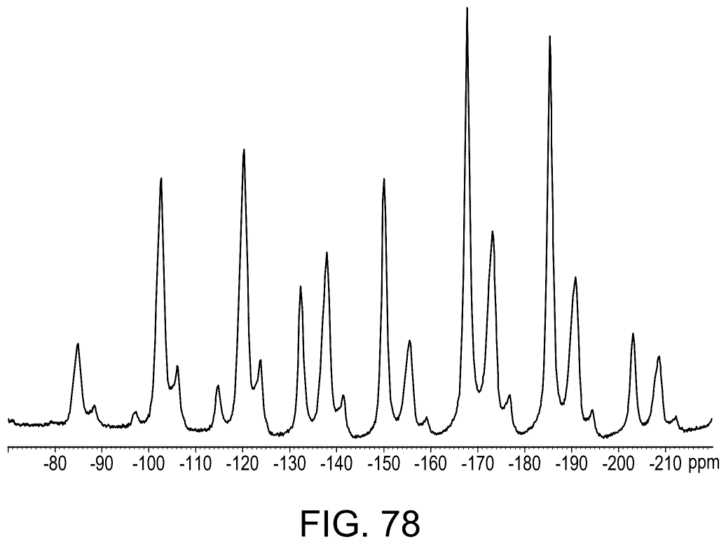

The ¹⁹F ssNMR spectrum of Form 1E is shown in FIG. 78.

The ¹³C ssNMR of Form 1E is shown in FIG. 5A with the peaks listed in Table 4. A comparison of the ¹³C ssNMR of Form 1E and Compound A free base hydrate is shown in FIG. 5B. The ¹³C ssNMR was acquired using the CPTOSS sequence and peaks were picked using TopSpin 3.6.4.

TABLE 4
¹³C ssNMR peaks of Form 1E

| Peak | v(F1) [ppm] | Intensity [abs] |
|---|---|---|
| 1 | 176.4 | 4802417.6 |
| 2 | 175.9 | 3290163.2 |
| 3 | 163.2 | 1748491.3 |
| 4 | 161.6 | 1816937.2 |
| 5 | 154.5 | 2911313.6 |
| 6 | 152.3 | 248237.0 |
| 7 | 150.2 | 489186.3 |
| 8 | 149.4 | 781849.3 |
| 9 | 147.9 | 2975975.1 |
| 10 | 143.0 | 1488580.8 |
| 11 | 142.4 | 1364903.4 |
| 12 | 134.0 | 790007.4 |
| 13 | 131.0 | 1682164.4 |
| 14 | 125.3 | 1212967.2 |
| 15 | 121.8 | 2260367.7 |
| 16 | 118.4 | 2310883.5 |
| 17 | 110.6 | 2188948.7 |
| 18 | 94.2 | 876584.7 |
| 19 | 92.7 | 442832.2 |
| 20 | 80.4 | 3457865.6 |
| 21 | 78.7 | 1877489.7 |
| 22 | 70.1 | 1498341.6 |
| 23 | 61.7 | 903498.4 |
| 24 | 56.3 | 919530.8 |
| 25 | 54.6 | 1116079.3 |
| 26 | 41.9 | 569652.9 |
| 27 | 39.4 | 1011405.2 |
| 28 | 35.9 | 173920.3 |
| 29 | 31.9 | 374409.4 |
| 30 | 27.1 | 382388.7 |
| 31 | 22.6 | 360466.2 |

The comparative solubilities of Form 1 and Form 1E are shown in Tables 5 and 6.

TABLE 5
Form 1 Solubility

| Sample | Conc (mg/mL) | pH | Equilibrium crystal form |
|---|---|---|---|
| FaSSGF | 4.42 | 2.06 | Form 1 |
| FaSSIF | 0.035 | 7.15 | Form 1 |
| FeSSIF | 0.56 | 5.51 | Form 1 |
| PBS | 0.01 | 7.04 | Form 1 |
| H₂O | 0.0003 | 8.73 | Form 1 |

TABLE 6
Form 1E Solubility

| Sample | Conc (mg/mL) | pH | Equilibrium crystal form |
|---|---|---|---|
| FaSSGF | 5.99 | 1.99 | Form 1E |
| FaSSIF | 1.73 | 4.86 | Form 1E |
| FeSSIF | 2.50 | 4.58 | Form 1E |
| PBS | 1.30 | 4.55 | Form 1E |
| H₂O | 7.76 | 3.93 | Form 1E |

As shown in Tables 7 and 8, Form 1E exhibited higher solubility in several media relative to the Form 1.

The solubility of Compound A malonate salt anhydrous (Form 1E) was further evaluated. Form 1E exhibited a 5-10 times increase in solubility relative to Form 1. In addition, Form 1E was shown to be physically stable under accelerated conditions and was shown to be chemically stable at 5° C. for more than 2 years.

The pharmacokinetic (PK) properties of Form 1E was evaluated in dogs. As shown in FIG. 5C, Form 1E exhibited approximately a 2× improvement in plasma concentration based on single dose dog PK compared to Compound A free base.

The PK properties of Form 1E was evaluated in male dogs under fasted and fed conditions. The data is shown in Table 7.

TABLE 7

| | Dose | | | AUC_{∞} | AUCₜ | CL/F | Cmax | MRT | Tₘₐₓ | t_{1/2, z} |
|---|---|---|---|---|---|---|---|---|---|---|
| Study | (mg/kg) | Vehicle | Treatment | (uM*h) | (uM*h) | (L/h/kg) | (uM) | (h) | (h) | (h) |
| A | 30.0 | 1 | Fasted | 14.2 | 14.1 | 4.41 | 5.82 | 3.16 | 0.312 | 2.67 |
| | 30.0 | 1 | Fed | 17 | 17 | 3.35 | 4.29 | 4.14 | 1.62 | 2.96 |
| B | 50.0 | 2 | Fasted | 3.54 | 3.53 | 47 | 1.35 | 3.79 | 0.9 | 3.47 |
| | 50.0 | 2 | Fed | 3.91 | 3.89 | 69.5 | 1.05 | 4.26 | 1.5 | 4.83 |
| | | | (high fat) | | | | | | | |

Vehicle 1: 0.1 Tween80, 2% HPMC, 97.9% water/malonic acid (pH 2.0)
Vehicle 2: 0.1 Tween80, 2% HPMC, 97.9% water/MSA (pH7.0)

Form 1E was administered (p.o.) at 30 mg/kg under fasted or fed conditions, wherein the animals were fed with kibble instead of high fat diet to reduce emesis and administered Form 1E after 2.5 h after food offering. Animals in prior studies using 50 mg/kg of Form 1E and a high fat diet suffered from emesis. As shown in FIGS. 5D and 5E, the plasma exposure did not show a difference between fed and fasted conditions regarding to Cₘₐₓ and AUC.

The DVS isotherm plot of Form 1E is shown in FIG. 6 and shows a 0.6 wt. % moisture uptake at 90% relative humidity indicating that Form 1E is non-hygroscopic.

### Compound A Maleate Salt Hydrate (“Form 1A”)

The DSC thermograph of Form 1A is shown in FIG. 8 and shows a first endothermic event was observed below 100° C. corresponding to loss of solvent. Form 1A did not have a melting point and exhibited degradation at 170° C. with an exothermic event corresponding to weight loss in TGA.

The TGA thermograph of Form 1A is shown in FIG. 9. The TGA shows a 1.782% wt. loss was observed below 150° C. corresponding to the broad endothermic event observed in DSC analysis and assigned as solvent loss. Form 1A started degrading at 170° C. with weight loss in TGA, which corresponds to exothermic event observed during DSC analysis. A 7.21 wt. % loss was observed between 170° C. and 200° C. which is believed to correspond to a hemimaleate salt (theoretical 8.13 wt. % loss).

The XRPD of Form 1A is shown in FIG. 7 having the peaks listed in Table 8.

TABLE 8
XRPD peaks of Form 1A

| Pos. [°2θ] | d-spacing [Å] | Rel. Int. [%] |
|---|---|---|
| 7.5 | 11.83 | 14.82 |
| 8.1 | 10.93 | 76.94 |
| 8.5 | 10.37 | 100 |
| 10.2 | 8.64 | 4.40 |
| 12.7 | 7.00 | 20.58 |
| 13.3 | 6.64 | 2.79 |
| 14.5 | 6.12 | 76.44 |
| 15.0 | 5.91 | 20.75 |
| 15.3 | 5.80 | 28.7 |
| 15.7 | 5.65 | 60.63 |
| 16.2 | 5.48 | 10.24 |
| 16.7 | 5.30 | 34.7 |
| 17.3 | 5.11 | 12.69 |
| 18.2 | 4.88 | 19.97 |
| 18.7 | 4.75 | 21.59 |
| 19.2 | 4.63 | 10.02 |
| 19.8 | 4.48 | 28.38 |
| 20.5 | 4.32 | 53.87 |
| 22.0 | 4.05 | 77.68 |
| 22.6 | 3.93 | 23.58 |
| 23.5 | 3.79 | 24.37 |
| 23.9 | 3.73 | 13.52 |
| 24.3 | 3.66 | 16.10 |
| 24.6 | 3.62 | 22.8 |
| 25.4 | 3.51 | 48.33 |
| 26.7 | 3.34 | 57.02 |
| 27.6 | 3.23 | 6.69 |
| 28.5 | 3.14 | 16.71 |
| 29.3 | 3.05 | 22.93 |
| 29.8 | 3.00 | 11.52 |
| 30.5 | 2.94 | 8.14 |
| 30.9 | 2.89 | 3.69 |
| 31.7 | 2.82 | 1.19 |
| 32.6 | 2.75 | 5.45 |
| 32.9 | 2.72 | 8.36 |
| 33.7 | 2.66 | 6.38 |
| 34.1 | 2.63 | 4.09 |
| 34.8 | 2.58 | 5.86 |
| 35.9 | 2.50 | 5.17 |
| 36.3 | 2.47 | 4.29 |
| 37.3 | 2.41 | 1.87 |
| 38.0 | 2.37 | 1.24 |
| 39.3 | 2.29 | 3.77 |
| 40.1 | 2.25 | 1.73 |
| 41.4 | 2.18 | 4.85 |
| 42.6 | 2.12 | 3.55 |

The ¹H NMR spectrum of Form 1A is shown in FIG. 10 and shows the Compound A:maleic acid stoichiometry to be 1:1.

The DVS isotherm of Form 1A is shown in FIG. 11 and shows a 5.4 wt. % moisture uptake at 95% relative humidity indicating Form 1A to be hygroscopic.

### Compound A Quercetin Co-Crystal (“Form C-P”)

The DSC thermograph of Form C-P is shown in FIG. 32 indicating a first endotherm below 100° C. corresponding to the loss of solvent (DCM:MeOH) and a second endotherm at 212.4° C. indicating decomposition of Form C-P as per observed weight loss in TGA.

The TGA thermograph of Form C-P is shown in FIG. 14 indicating a 1.72% weight loss up to 160° C. corresponding to solvent loss (DCM:MeOH). A second weight loss above 160° C. was observed indicating decomposition of Form C-P.

The XRPD of Form C-P is shown in FIG. 31 having the peaks listed in Table 9.

TABLE 9
XRPD peaks of Form C-P

| Pos. [°2θ] | d-spacing [Å] | Rel. Int. [%] |
|---|---|---|
| 4.5 | 19.57 | 28.03 |
| 4.9 | 17.97 | 47.32 |
| 7.0 | 12.69 | 100 |
| 8.9 | 9.91 | 22.29 |
| 9.5 | 9.31 | 67.98 |
| 9.8 | 9.02 | 38.54 |
| 11.8 | 7.50 | 45.17 |
| 13.1 | 6.77 | 33.3 |
| 13.6 | 6.49 | 3.99 |
| 14.8 | 5.98 | 6.32 |
| 15.4 | 5.75 | 25.67 |
| 16.9 | 5.24 | 41.88 |
| 17.8 | 4.97 | 23.19 |
| 19.1 | 4.66 | 69.81 |
| 19.6 | 4.52 | 37.97 |
| 20.3 | 4.37 | 16.13 |
| 20.9 | 4.25 | 15.78 |
| 22.1 | 4.01 | 5.47 |
| 23.1 | 3.85 | 25.29 |
| 23.7 | 3.75 | 10.80 |
| 24.8 | 3.59 | 14.73 |
| 25.6 | 3.48 | 5.50 |
| 26.0 | 3.42 | 9.96 |
| 27.2 | 3.28 | 5.59 |
| 27.9 | 3.20 | 6.64 |
| 28.6 | 3.12 | 4.39 |
| 29.8 | 3.00 | 5.50 |
| 30.6 | 2.92 | 3.66 |
| 32.1 | 2.79 | 2.66 |
| 36.7 | 2.45 | 1.93 |

The DVS isotherm of Form C-P is shown in FIG. 34 indicating a 3.08 wt. % moisture uptake at 80% RH indicating that Form C-P is hygroscopic.

### Compound A Citrate Salt Hydrate (“Form 1B”)

The DSC thermograph of Form 1B is depicted in FIG. 13 showing a first endotherm below 100° C. corresponding to the loss of water. The endotherm(s) above 150° C. correspond to decomposition of the salt Form 1B.

The TGA thermograph of Form 1B is shown in FIG. 14 and shows a 2.87% wt. loss (between 1-2 molar equivalent of water) below 150° C. A 29.838 wt. % loss was observed between 15° and 250° C. (between 1-2 molar equivalent citric acid, theoretical 22.65 wt. % loss) maybe concurrent to the degradation of Form 1B. The XRPD of Form 1B is shown in FIG. 12 having the peaks listed in Table 10.

TABLE 10
XRPD peaks of Form 1B

| Pos. [°2θ] | d-spacing [Å] | Rel. Int. [%] |
|---|---|---|
| 4.5 | 19.53 | 4.94 |
| 7.8 | 11.33 | 43.53 |
| 9.3 | 9.55 | 3.91 |
| 10.6 | 8.37 | 3.24 |
| 12.8 | 6.89 | 57.75 |
| 14.8 | 5.97 | 13.23 |
| 15.5 | 5.70 | 12.47 |
| 17.4 | 5.09 | 27.67 |
| 18.5 | 4.78 | 49.13* |
| 19.9 | 4.47 | 100 |
| 21.3 | 4.16 | 15.36 |
| 23.2 | 3.84 | 5.69 |
| 25.3 | 3.52 | 23.85 |
| 26.6 | 3.36 | 11.27 |
| 27.1 | 3.29 | 9.83 |
| 29.2 | 3.06 | 10.55 |
| 33.7 | 2.66 | 2.43 |

The ¹H NMR spectrum of Form 1B is shown in FIG. 15 indicating a Compound A:citric acid stoichiometry of 1:1.

The DVS isotherm of Form 1B is shown in FIG. 16 and shows a 4.7 wt. % moisture uptake at 95% RH indicating Form 1B is hygroscopic.

### Compound A Hydrobromide (“Form S-L”)

The DSC thermograph of Form S-L is shown in FIG. 18 indicating a first endotherm below 100° C. corresponding to the loss of solvent (ACN:MeOH) and a second broad exothermic event associated with degradation.

The XRPD of Form S-L is shown in FIG. 17 having the peaks listed in Table 11.

TABLE 11
XRPD peaks of Form S-L

| Pos. [°2θ] | d-spacing [Å] | Rel. Int. [%] |
|---|---|---|
| 4.9 | 17.85 | 60.49 |
| 6.5 | 13.61 | 48.39 |
| 7.0 | 12.65 | 100* |
| 8.0 | 11.08 | 72.54 |
| 9.2 | 9.57 | 25.15 |
| 10.3 | 8.62 | 7.38 |
| 11.0 | 8.02 | 4.80 |
| 12.2 | 7.26 | 15.03 |
| 14.5 | 6.10 | 11.74 |
| 15.3 | 5.77 | 10.03 |
| 16.9 | 5.24 | 11.97 |
| 17.9 | 4.97 | 35.86 |
| 19.1 | 4.66 | 10.61 |
| 19.6 | 4.53 | 21.41 |
| 20.3 | 4.38 | 13.69 |
| 21.2 | 4.19 | 13.82 |
| 22.2 | 4.00 | 8.85 |
| 23.1 | 3.84 | 16.13 |
| 24.8 | 3.58 | 11.81 |
| 26.8 | 3.32 | 4.30 |
| 28.1 | 3.17 | 5.63 |

The ¹H NMR spectrum of Form S-L is shown in FIG. 19.

### Compound A Hydrochloride (“Form S-T”)

The DSC thermograph of Form S-T is shown in FIG. 21 indicating a first endotherm below 100° C. corresponding to the loss of solvent (MeOH) and a second endothermic event associated with decomposition of Form S-T as per observed weight loss in TGA.

The TGA thermograph of Form S-T is shown in FIG. 22 indicating a 5.241% wt loss (between 2-3 molar equivalent of water) below 150° C. A 6.806 wt. % loss was observed from 150 to 250° C. (between 1-2 molar equivalent hydrochloric acid). It is believed that the weight loss corresponds to the concomitant degradation of Form S-T.

The XRPD of Form S-T is shown in FIG. 20 having the peaks listed in Table 12.

TABLE 12
XRPD peaks of Form S-T

| Pos. [°2θ] | d-spacing [Å] | Rel. Int. [%] |
|---|---|---|
| 4.6 | 19.04 | 28.76 |
| 6.6 | 13.37 | 43.07 |
| 8.1 | 10.88 | 100 |
| 9.4 | 9.40 | 20.76 |
| 10.5 | 8.46 | 24.93 |
| 12.1 | 7.33 | 33.87 |
| 12.4 | 7.13 | 23.21 |
| 13.1 | 6.74 | 10.35 |
| 14.0 | 6.33 | 3.67 |
| 14.5 | 6.10 | 17.09 |
| 14.8 | 5.98 | 18.66 |
| 15.5 | 5.73 | 25.33 |
| 16.3 | 5.45 | 14.10 |
| 16.8 | 5.27 | 23.31 |
| 18.1 | 4.90 | 39.78 |
| 19.3 | 4.59 | 20.32 |
| 19.7 | 4.50 | 42.88 |
| 20.5 | 4.34 | 51.07 |
| 20.9 | 4.24 | 20.72 |
| 21.5 | 4.14 | 39.84 |
| 22.5 | 3.95 | 18.11 |
| 23.0 | 3.87 | 14.49 |
| 23.5 | 3.79 | 20.8 |
| 23.9 | 3.72 | 22.55 |
| 24.3 | 3.66 | 18.53 |
| 24.8 | 3.58 | 13.96 |
| 25.5 | 3.49 | 11.75 |
| 26.2 | 3.40 | 21.15 |
| 27.0 | 3.30 | 29.73 |
| 27.8 | 3.21 | 31.57 |
| 28.6 | 3.12 | 19.75 |
| 30.6 | 2.92 | 10.35 |
| 31.6 | 2.83 | 5.11 |
| 33.1 | 2.71 | 6.51 |
| 34.4 | 2.61 | 3.31 |

### Compound A Edisylate Salt (“Form 1F”)

The DSC thermograph of Form 1F is shown in FIG. 25 indicating a first endotherm below 100° C. corresponding to loss of solvent (MeOH).

The TGA thermograph of Form 1F is shown in FIG. 26 indicating a 4.226% wt. loss (between 1-2 molar equivalent of water) below 150° C. and a 2.124 wt. % loss between 15° and 250° C. (less than 0.5 molar equivalent ethanedisulfonic acid).

The XRPD of Form 1F is shown in FIG. 24 having the peaks listed in Table 13.

TABLE 13
XRPD peaks of Form 1F

| Pos. [°2θ] | d-spacing [Å] | Rel. Int. [%] |
|---|---|---|
| 5.4 | 16.47 | 22.44 |
| 6.3 | 14.07 | 100.00 |
| 7.8 | 11.27 | 5.64 |
| 9.8 | 8.99 | 13.12 |
| 10.7 | 8.29 | 30.21 |
| 11.3 | 7.85 | 33.12 |
| 12.5 | 7.07 | 29.88 |
| 13.3 | 6.65 | 33.83 |
| 14.5 | 6.09 | 11.76 |
| 15.4 | 5.74 | 12.23 |
| 16.0 | 5.53 | 8.19 |
| 16.4 | 5.41 | 16.42 |
| 17.1 | 5.19 | 60.62 |
| 17.5 | 5.06 | 25.95 |
| 18.8 | 4.72 | 23.68 |
| 19.2 | 4.63 | 42.48 |
| 19.5 | 4.56 | 25.33 |
| 20.0 | 4.44 | 53.27 |
| 20.9 | 4.25 | 23.21 |
| 21.4 | 4.16 | 28.57 |
| 22.6 | 3.94 | 13.60 |
| 23.1 | 3.85 | 10.74 |
| 24.2 | 3.68 | 14.28 |
| 24.9 | 3.58 | 20.3* |
| 25.3 | 3.52 | 18.67 |
| 26.2 | 3.40 | 18.46 |
| 26.8 | 3.33 | 14.03 |
| 27.9 | 3.20 | 23.25 |
| 28.5 | 3.13 | 21.96 |
| 29.8 | 3.00 | 16.95 |
| 30.3 | 2.95 | 6.54 |
| 31.1 | 2.88 | 5.60 |
| 31.9 | 2.80 | 5.20 |
| 33.9 | 2.65 | 4.24 |
| 34.3 | 2.61 | 6.20 |
| 35.5 | 2.53 | 2.30 |
| 36.1 | 2.49 | 1.64 |
| 36.9 | 2.44 | 2.24 |
| 37.8 | 2.38 | 2.27 |
| 38.9 | 2.31 | 6.22 |
| 43.4 | 2.08 | 3.92 |
| 44.2 | 2.05 | 2.20 |

### Compound A Hemiedisylate Salt (“Form 1G”)

The DSC thermograph of Form 1G is shown in FIG. 28 indicating a first endotherm below 100° C. corresponding to the loss of solvent (MeOH) and a second endotherm having an onset of 250° C. corresponding to decomposition.

The TGA thermograph of Form 1G is shown in FIG. 29 indicating a 1.948% wt. loss (between 0.5-1 molar equivalent of water) below 150° C. and a 8.644 wt. % loss between 150 and 300° C. (less than 0.5 molar equivalent ethane disulfonic acid).

The XRPD of Form 1G is shown in FIG. 27 having the peaks listed in Table 14.

TABLE 14
XRPD Peaks of Form 1G

| Pos. [°2θ] | d-spacing [Å] | Rel. Int. [%] |
|---|---|---|
| 5.0 | 17.69 | 51.46 |
| 6.2 | 14.16 | 66.75 |
| 6.7 | 13.24 | 89.65 |
| 7.0 | 12.57 | 62.24 |
| 9.5 | 9.33 | 28.27 |
| 10.6 | 8.32 | 29.37 |
| 11.2 | 7.88 | 15.51 |
| 12.9 | 6.89 | 49.14 |
| 13.7 | 6.47 | 100 |
| 14.3 | 6.18 | 67.34 |
| 14.9 | 5.95 | 75.61 |
| 15.3 | 5.78 | 18.38 |
| 17.0 | 5.21 | 49.26 |
| 17.3 | 5.14 | 76.02 |
| 17.9 | 4.95 | 11.59 |
| 18.4 | 4.82 | 30.66 |
| 19.2 | 4.63 | 37.98 |
| 19.7 | 4.50 | 26.06 |
| 20.5 | 4.34 | 77.84 |
| 21.0 | 4.23 | 22.58 |
| 21.8 | 4.08 | 63.9 |
| 22.1 | 4.02 | 13.15 |
| 22.8 | 3.89 | 21.26 |
| 23.2 | 3.83 | 16.24 |
| 23.7 | 3.75 | 8.88 |
| 24.6 | 3.61 | 8.31 |
| 24.9 | 3.57 | 13.80 |
| 25.3 | 3.52 | 10.37 |
| 26.8 | 3.32 | 6.26 |
| 27.3 | 3.27 | 13.98 |
| 28.3 | 3.15 | 2.53 |
| 31.7 | 2.82 | 11.05 |
| 37.4 | 2.40 | 6.32 |
| 44.2 | 2.05 | 4.84 |

The ¹H NMR of Form 1G is shown in FIG. 30 indicating a Compound A:ethanedisulfonic acid stoichiometry of 1:0.5.

### Compound A Glycine Hydrochloride Co-Crystal Hydrate (“Form C1”)

The DSC thermograph of Form C1 is shown in FIG. 36 indicating no melting point and degradation at 200° C., wherein the exothermic event corresponds to the observed weight loss in the TGA.

The TGA thermograph of Form C1 is shown in FIG. 37 indicating a 4.294% wt. loss (between 1-2 molar equivalent of water) below 200° C. and a 24.041 wt. % loss between 20° and 300° C. (between 1-2 molar equivalent glycine HCl, theoretical 14.53 wt. % loss). Form C1 began degrading at 200° C. with weight loss in TGA, corresponding to the exothermic event observed during DSC analysis.

The XRPD data of Form C1 is shown in FIG. 35 having the peaks listed in Table 15.

TABLE 15
XRPD peaks of Form C1

| Pos. [°2θ] | d-spacing [Å] | Rel. Int. [%] |
|---|---|---|
| 4.7 | 18.72 | 25.68 |
| 6.6 | 13.37 | 31.01 |
| 8.1 | 10.92 | 100 |
| 9.4 | 9.43 | 17.26 |
| 10.5 | 8.44 | 25.05 |
| 11.5 | 7.73 | 6.14 |
| 12.0 | 7.35 | 37.72 |
| 12.4 | 7.13 | 24.32 |
| 13.1 | 6.75 | 15.66 |
| 14.1 | 6.29 | 3.93 |
| 14.5 | 6.10 | 18.18 |
| 14.8 | 5.99 | 17.99 |
| 15.4 | 5.76 | 20.42 |
| 16.3 | 5.45 | 14.29 |
| 16.6 | 5.34 | 13.02 |
| 16.8 | 5.27 | 17.23 |
| 18.0 | 4.94 | 76.47 |
| 19.0 | 4.66 | 7.05 |
| 19.3 | 4.60 | 13.41 |
| 19.7 | 4.51 | 31.5 |
| 20.5 | 4.34 | 36.82 |
| 20.9 | 4.24 | 13.40 |
| 21.5 | 4.13 | 28.63 |
| 21.8 | 4.07 | 9.57 |
| 22.5 | 3.95 | 8.92 |
| 22.9 | 3.88 | 14.51 |
| 23.5 | 3.79 | 14.25 |
| 23.8 | 3.74 | 43.81 |
| 24.4 | 3.65 | 7.30 |
| 24.9 | 3.58 | 7.37 |
| 25.5 | 3.49 | 6.94 |
| 26.2 | 3.40 | 12.80 |
| 27.0 | 3.30 | 20.14 |
| 27.8 | 3.21 | 21.69 |
| 28.5 | 3.13 | 36.22 |
| 30.3 | 2.95 | 5.81 |
| 31.1 | 2.87 | 10.34 |
| 31.6 | 2.83 | 4.98 |
| 33.1 | 2.70 | 5.88 |
| 33.9 | 2.64 | 7.98 |
| 34.5 | 2.60 | 9.14 |
| 35.4 | 2.53 | 3.45 |
| 36.3 | 2.47 | 10.26 |
| 36.7 | 2.45 | 19.35 |
| 39.2 | 2.30 | 7.14 |

The ¹H NMR of Form C1 is shown in FIG. 38 indicating a Compound A:glycine hydrochloride stoichiometry of 1:1.

The DVS isotherm of Form C1 is shown in FIG. 39 indicating a 4.0 wt. % moisture uptake at 85% relative humidity followed by deliquescence at 95% relative humidity indicating Form C1 is hygroscopic.

### Compound A Acetazolamide Co-Crystal (“Form C-E”)

The XRPD of Form C-E is shown in FIG. 40 having the peaks listed in Table 16.

TABLE 16
XRPD peaks of Form C-E

| Pos. [°2θ] | d-spacing [Å] | Rel. Int. [%] |
|---|---|---|
| 5.0 | 17.78 | 80.98 |
| 6.3 | 14.08 | 39.9 |
| 7.0 | 12.62 | 100 |
| 9.5 | 9.26 | 15.31 |
| 11.4 | 7.74 | 28.42 |
| 11.9 | 7.45 | 17.30 |
| 12.8 | 6.93 | 10.92 |
| 15.4 | 5.74 | 23.7 |
| 17.0 | 5.22 | 41.31 |
| 17.3 | 5.11 | 29.91 |
| 17.9 | 4.95 | 43.71 |
| 18.7 | 4.75 | 41.34 |
| 19.1 | 4.66 | 39.18 |
| 19.7 | 4.52 | 51.05 |
| 20.3 | 4.38 | 35.7 |
| 21.4 | 4.15 | 34.29 |
| 22.8 | 3.91 | 35.04 |
| 23.2 | 3.84 | 55.05 |
| 23.8 | 3.75 | 34.34 |
| 24.9 | 3.58 | 35.99 |
| 27.6 | 3.23 | 17.01 |
| 28.9 | 3.08 | 16.34 |
| 29.8 | 3.00 | 18.65 |
| 30.8 | 2.91 | 12.42 |
| 32.1 | 2.79 | 8.23 |

### Compound A L-Tartaric Acid Co-Crystal (“Form C-D”)

The XRPD of Form C-D is shown in FIG. 41 having the peaks listed in Table 17.

TABLE 17
XRPD peaks of Form C-D

| Pos. [°2θ] | d-spacing [Å] | Rel. Int. [%] |
|---|---|---|
| 8.1 | 10.94 | 8.24 |
| 8.3 | 10.59 | 14.42 |
| 10.3 | 8.62 | 2.02 |
| 10.7 | 8.25 | 5.55 |
| 12.5 | 7.11 | 4.83 |
| 14.1 | 6.27 | 100 |
| 15.3 | 5.79 | 15.52 |
| 16.2 | 5.48 | 21.62 |
| 16.8 | 5.27 | 29.17 |
| 17.1 | 5.18 | 65.07 |
| 17.5 | 5.07 | 22.64 |
| 17.7 | 5.02 | 38.0 |
| 17.8 | 4.98 | 27.54 |
| 19.0 | 4.67 | 21.43 |
| 19.3 | 4.60 | 31.7 |
| 20.5 | 4.34 | 3.36 |
| 21.5 | 4.13 | 7.94 |
| 22.2 | 4.00 | 81.62 |
| 23.4 | 3.81 | 9.34 |
| 24.3 | 3.66 | 16.95 |
| 24.6 | 3.61 | 49.99 |
| 25.0 | 3.56 | 23.43 |
| 25.5 | 3.49 | 7.51 |
| 27.5 | 3.24 | 6.38 |
| 27.9 | 3.20 | 6.14 |
| 28.7 | 3.11 | 1.67 |
| 29.3 | 3.05 | 8.44 |
| 31.7 | 2.82 | 60.89 |
| 35.5 | 2.53 | 38.93 |
| 37.2 | 2.42 | 5.49 |
| 41.6 | 2.17 | 1.15 |
| 44.0 | 2.06 | 4.03 |
| 44.4 | 2.04 | 11.16 |

### Compound A Glutaric Acid Co-Crystal (“Form C-C”)

The XRPD of Form C-C is shown in FIG. 42 having the peaks listed in Table 18.

TABLE 18
XRPD peaks of Form C-C

| Pos. [°2θ] | d-spacing [Å] | Rel. Int. [%] |
|---|---|---|
| 6.3 | 14.04 | 100 |
| 8.2 | 10.79 | 12.21 |
| 8.4 | 10.57 | 17.66 |
| 8.7 | 10.11 | 9.90 |
| 9.0 | 9.82 | 11.89 |
| 10.7 | 8.29 | 1.89 |
| 11.2 | 7.89 | 15.58 |
| 12.2 | 7.23 | 2.82 |
| 13.9 | 6.39 | 50.46 |
| 14.2 | 6.25 | 35.6 |
| 15.0 | 5.90 | 10.00 |
| 15.9 | 5.58 | 16.66 |
| 16.8 | 5.27 | 65.23 |
| 17.2 | 5.17 | 29.97 |
| 17.5 | 5.08 | 31.07 |
| 17.8 | 4.98 | 13.53 |
| 19.0 | 4.68 | 47.7 |
| 19.4 | 4.58 | 12.68 |
| 20.2 | 4.40 | 8.77 |
| 21.2 | 4.20 | 10.20 |
| 22.3 | 3.98 | 7.11 |
| 23.1 | 3.84 | 6.49 |
| 23.9 | 3.72 | 6.78 |
| 24.3 | 3.66 | 27.25 |
| 24.6 | 3.62 | 43.27 |
| 25.1 | 3.55 | 11.33 |
| 25.4 | 3.51 | 8.68 |
| 26.0 | 3.43 | 5.71 |
| 27.1 | 3.29 | 3.94 |
| 27.4 | 3.25 | 8.47 |
| 28.6 | 3.12 | 4.47 |
| 29.4 | 3.04 | 1.28 |
| 30.4 | 2.94 | 1.82 |
| 33.2 | 2.70 | 3.27 |
| 33.6 | 2.67 | 2.51 |
| 34.4 | 2.61 | 2.08 |
| 35.2 | 2.55 | 1.99 |
| 44.2 | 2.05 | 4.49 |
| 44.5 | 2.04 | 14.81 |

### Compound A Free Base Hydrate (“Form 1”)

The DSC thermograph of Form 1 is shown in FIG. 44 and shows an endothermic event during the first heating cycle corresponding to solvent/water loss with weight loss observed in TGA below 150° C. The absence of an endothermic event in the second heating cycle below 150° C. and the exothermic event at 262.58° C. corresponds to degradation with concurrent melting as supported by significant observed weight loss in TGA.

The TGA thermograph of Form 1 is shown in FIG. 45 and shows a 2.67% weight loss (1 molar equivalent of water) below 150° C. and degradation upon melting above 263° C.

The XRPD of Form 1 is shown in FIG. 43 having the peaks in Table 19.

TABLE 19
XRPD peaks of Form 1

| Pos. [°2θ] | d-spacing [Å] | Rel. Int. [%] |
|---|---|---|
| 5.0 | 17.70 | 49.61 |
| 7.0 | 12.57 | 100 |
| 9.6 | 9.25 | 14.15 |
| 11.1 | 7.98 | 1.42 |
| 11.9 | 7.46 | 19.85 |
| 14.0 | 6.31 | 2.93 |
| 14.9 | 5.95 | 7.69 |
| 15.5 | 5.73 | 16.66 |
| 15.7 | 5.65 | 3.91 |
| 17.0 | 5.22 | 37.45 |
| 17.9 | 4.95 | 38.13 |
| 18.5 | 4.80 | 0.97 |
| 19.1 | 4.64 | 23.86 |
| 19.7 | 4.51 | 35.83 |
| 20.4 | 4.35 | 7.05 |
| 21.0 | 4.23 | 7.73 |
| 22.2 | 4.00 | 9.49 |
| 22.7 | 3.91 | 1.75 |
| 23.2 | 3.84 | 23.38 |
| 23.8 | 3.74 | 6.45 |
| 24.2 | 3.67 | 4.03 |
| 24.9 | 3.58 | 23.61 |
| 25.3 | 3.53 | 4.73 |
| 26.2 | 3.40 | 1.04 |
| 26.8 | 3.32 | 4.41 |
| 27.2 | 3.28 | 2.94 |
| 28.0 | 3.19 | 1.86 |
| 28.2 | 3.16 | 6.60 |
| 28.6 | 3.12 | 2.07 |
| 28.8 | 3.10 | 1.76 |
| 29.1 | 3.07 | 5.25 |
| 29.8 | 2.99 | 8.54 |
| 30.6 | 2.93 | 1.72 |
| 30.8 | 2.91 | 4.21 |
| 31.2 | 2.87 | 1.12 |
| 31.5 | 2.84 | 2.17 |
| 32.0 | 2.79 | 7.09 |
| 32.4 | 2.77 | 2.46 |
| 33.0 | 2.71 | 0.42 |
| 33.9 | 2.65 | 1.60 |
| 34.3 | 2.61 | 1.87 |
| 34.6 | 2.59 | 1.13 |
| 35.3 | 2.55 | 0.92 |
| 35.5 | 2.53 | 1.70 |
| 36.0 | 2.49 | 0.46 |
| 36.6 | 2.46 | 1.17 |
| 36.8 | 2.44 | 1.39 |
| 37.4 | 2.40 | 0.48 |
| 38.2 | 2.35 | 0.63 |
| 39.5 | 2.28 | 0.58 |

The ¹H NMR of Form 1 is shown in FIG. 46.

¹⁹F ssNMR of Compound A Free Base Hydrate (“Form 1”)

The ¹⁹F spectrum of Compound A was acquired by using the CPTOSS pulse sequence. Peaks were picked using TopSpin 3.6.4. The ¹³C spectrum is shown in FIG. 76. The peaks are listed in Table 22.

TABLE 22
¹⁹F peaks

| Peak | v(F1) [ppm] | Intensity [abs] |
|---|---|---|
| 1 | −137 | 211437.43 |
| 2 | −170 | 508099.94 |

¹³C ssNMR of Compound A Free Base Hydrate (“Form 1”)

The ¹³C spectrum of Compound A was acquired by using the CPTOSS pulse sequence. Peaks were picked using TopSpin 3.6.4. The ¹³C spectrum is shown in FIG. 76. The peaks are listed in Table 21.

TABLE 21
¹³C peaks

| Peak | v(F1) [ppm] | Intensity [abs] |
|---|---|---|
| 1 | 163.8 | 4282614.5 |
| 2 | 161.4 | 3457920.2 |
| 3 | 155.5 | 4732578.4 |
| 4 | 152.0 | 479947.7 |
| 5 | 149.9 | 911342.3 |
| 6 | 149.3 | 1633703.5 |
| 7 | 142.1 | 6149153.1 |
| 8 | 139.5 | 5379085.4 |
| 9 | 135.8 | 3448900.6 |
| 10 | 131.9 | 1946662.6 |
| 11 | 128.9 | 6897035.1 |
| 12 | 122.9 | 4967685.7 |
| 13 | 113.2 | 4927154.6 |
| 14 | 110.2 | 4970301.8 |
| 15 | 97.6 | 1889539.7 |
| 16 | 96.1 | 1424696.6 |
| 17 | 77.8 | 3905761.2 |
| 18 | 75.2 | 2583327.2 |
| 19 | 73.2 | 9357810.9 |
| 20 | 69.8 | 1652581.9 |
| 21 | 66.3 | 1937960.7 |
| 22 | 59.4 | 2439431.4 |
| 23 | 55.2 | 3545256.8 |
| 24 | 53.4 | 1411137.5 |
| 25 | 41.5 | 1775977.2 |
| 26 | 38.0 | 2341432.7 |
| 27 | 28.4 | 644822.0 |
| 28 | 25.3 | 3797861.3 |

### Compound A Free Base Hydrate (“Form 2”)

The TGA thermograph of Form 2 is shown in FIG. 48 and shows a 5.554% wt. loss (2-3 molar equivalent of water) below 150° C. and shows degradation upon melting above 250° C.

The XRPD of Form 2 is shown in FIG. 47 having the peaks listed in Table 20.

TABLE 20
XRPD peaks of Form 2

| Pos. [°2θ] | d-spacing [Å] | Rel. Int. [%] |
|---|---|---|
| 4.9 | 17.95 | 27.66 |
| 5.2 | 17.12 | 23.87 |
| 7.0 | 12.66 | 86.53 |
| 7.2 | 12.28 | 40.55 |
| 9.7 | 9.08 | 14.80 |
| 12.0 | 7.36 | 21.3 |
| 14.0 | 6.33 | 2.92 |
| 15.1 | 5.88 | 14.24 |
| 15.6 | 5.68 | 34.99 |
| 17.2 | 5.16 | 62.09 |
| 17.9 | 4.97 | 21.98 |
| 18.1 | 4.91 | 100 |
| 18.7 | 4.76 | 3.76 |
| 19.3 | 4.60 | 71.84 |
| 19.9 | 4.47 | 98.74 |
| 20.5 | 4.33 | 21.06 |
| 21.1 | 4.20 | 23.29 |
| 22.4 | 3.97 | 25.48 |
| 22.9 | 3.89 | 8.06 |
| 23.4 | 3.81 | 86.11 |
| 23.9 | 3.72 | 25.47 |
| 24.0 | 3.71 | 24.68 |
| 24.4 | 3.64 | 15.74 |
| 24.8 | 3.58 | 29.77 |
| 25.1 | 3.55 | 60.95 |
| 25.4 | 3.51 | 24.01 |
| 25.5 | 3.50 | 23.11 |
| 26.4 | 3.37 | 7.35 |
| 27.0 | 3.30 | 22.96 |
| 27.3 | 3.26 | 21.35 |
| 28.2 | 3.17 | 25.34 |
| 28.4 | 3.14 | 20.01 |
| 28.8 | 3.10 | 14.68 |
| 29.0 | 3.07 | 22.8 |
| 29.3 | 3.05 | 21.4 |
| 30.0 | 2.97 | 42.59 |
| 30.9 | 2.89 | 31.09 |
| 31.6 | 2.83 | 17.14 |
| 32.1 | 2.78 | 26.67 |
| 32.5 | 2.75 | 19.22 |
| 33.1 | 2.70 | 7.69 |
| 34.0 | 2.63 | 9.85 |
| 34.5 | 2.60 | 12.36 |
| 34.8 | 2.58 | 9.41 |
| 35.4 | 2.53 | 11.85 |
| 35.6 | 2.52 | 10.29 |
| 36.2 | 2.48 | 6.76 |
| 36.9 | 2.43 | 13.99 |
| 37.5 | 2.39 | 5.56 |
| 38.4 | 2.34 | 7.22 |
| 39.6 | 2.27 | 6.91 |

### Amorphous Compound A Oxalate (“Form A1”)

The DSC thermograph of Form A1 is shown in FIG. 49 indicating a first endotherm below 100° C. corresponding to the loss of solvent (ACN:MeOH) and a second endotherm(s) above 150° C. corresponding to decomposition of the salt.

The TGA thermograph of Form A1 is shown in FIG. 50 and indicates a 4.698% wt. loss (between 1-2 molar equivalent of water) below 150° C. and a 16.708 wt. % loss between 15° and 250° C. (between 1-2 molar equivalent oxalic acid). Without wishing to be bound to any particular theory, the results indicate concomitant degradation of oxalate and Compound A.

The ¹H NMR of Form A1 is shown in FIG. 51.

The XRPD of Form A1 is shown in FIG. 52.

### Amorphous Compound A Tosylate (“Form A1-A”)

The DSC thermograph of Form A1-A is shown in FIG. 53 indicating a first endotherm below 100° C. corresponding to the loss of solvent (ACN:MeOH). Form A1-A is hygroscopic and amorphous form as per TGA and XRPD analysis.

The TGA thermograph of Form A1-A is shown in FIG. 54 indicating a 4.265% wt. loss (between 1-2 molar equivalent of water) below 150° C. and a 3.373 wt. % loss between 15° and 250° C. (less than 0.5 molar equivalent p-toluenesulfonic acid).

The ¹H NMR of Form A1-A is shown in FIG. 55 and shows a Compound A:toluenesulfonic acid stoichiometry of 1:1.

The XRPD of Form A1-A is shown in FIG. 56.

### Amorphous Compound A Saccharinate (“Form A1-B”)

The DSC thermograph of Form A1-B is shown in FIG. 57 and indicates a first endotherm below 100° C. corresponding to the loss of solvent (DCM:MeOH) and a second exothermic peak observed at 175.14° C. and a third exothermic peak at 184.32° C., which corresponds to Tg and degradation as seen in TGA.

The TGA thermograph of Form A1-B is shown in FIG. 58 and shows a 4.592% wt. loss (between 1-2 molar equivalent of water) below 150° C. and a 2.049 wt. % loss from 150 to 250° C. (less than 0.5 molar equivalent saccharine).

The ¹H NMR of Form A1-B is shown in FIG. 59 and indicates a Compound A:saccharin stoichiometry of 1:1.

The XRPD of Form A1-B is shown in FIG. 60.

### Amorphous Compound A Citrate (“Form A1-C”)

The DSC thermograph of Form A1-C is shown in FIG. 61 and indicates a first endotherm below 100° C. corresponding to the loss of solvent (ACN) and a second broad endothermic event corresponding to degradation as seen in the TGA.

The TGA thermograph of Form A1-C is shown in FIG. 62 and shows a 5.79% wt loss (between 2-3 molar equivalent of water) below 150° C. and a 25.841 wt. % loss from 150 to 250° C. (between 1-2 molar equivalent citric acid) indicating degradation.

The ¹H NMR of Form A1-C is shown in FIG. 63 and indicates a Compound A:citric acid stoichiometry of 1:1.

The XRPD of Form A1-C is shown in FIG. 64.

### Amorphous Compound A Free Base (“Form A2”)

The DSC thermograph of Form A2 is shown in FIG. 65 and indicates a first endothermic event below 100° C. corresponding to the weight loss of solvent (ACN) and a second endothermic peak corresponding to decomposition as seen in the TGA weight loss.

The TGA of Form A2 is shown in FIG. 66 and indicates a 3.009% wt. loss (between 1-2 molar equivalent of water) below 150° C.

The XRPD of Form A2 is shown in FIG. 67.

### Amorphous Compound A Besylate (“Form A1-D”)

The DSC of Form A1-D is shown in FIG. 68. The DSC data shows a first endotherm below 100° C. corresponding to the loss of solvent (ACN) and a second endotherm above 150° C. indicating decomposition as TGA weight loss was observed.

The TGA of Form A1-D is shown in FIG. 69. The TGA data shows 3.429% wt. loss (between 1-2 molar equivalent of H₂O) below 150° C. A 2.326 wt % loss was observed between 15° and 250° C. The weight loss does not correspond to benzenesulfonic acid (theoretical 19.43 wt %).

The ¹H NMR of Form A1-D is shown in FIG. 70 and indicates a Compound A:benzenesulfonic acid stoichiometry of 1:1.

The XRPD of Form A1-D is shown in FIG. 71.

### Amorphous Compound A Esylate (“Form A1-E”)

The DSC thermograph of Form A1-E is shown in FIG. 72 and indicates a first endotherm below 100° C. corresponding to the loss of solvent (ACN) and a second endotherm onset of 150° C. corresponding to decomposition.

The TGA thermograph of Form A1-E is shown in FIG. 73 and shows a 5.506% wt loss (between 2-3 molar equivalent of water) below 150° C. A 3.499 wt % loss is observed between 15° and 250° C. (less than 0.5 molar equivalent ethanesulfonic acid). The weight loss can't correspond to ethanesulfonic acid (theoretical 14.37 wt %)

The ¹H NMR of Form A1-E is shown in FIG. 74 showing a Compound A:ethanesulfonic acid stoichiometry of 1:1.

The XRPD of Form A1-E is shown in FIG. 75. Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

### REFERENCES

Simanshu, D. K.; Nissley, D. V.; McCormick, F. RAS Proteins and Their Regulators in Human Disease. Cell 2017, 170, 17-33.
Cox, A. D.; Fesik, S. W.; Kimmelman, A. C.; Luo, J.; Der, C. J. Drugging the undruggable RAS: Mission possible? Nat Rev Drug Discov 2014, 13, 828-851.
O'Bryan, J. P. Pharmacological targeting of RAS: Recent success with direct inhibitors. Pharmacol Res 2019, 139, 503-511.
Ostrem, J. M.; Peters, U.; Sos, M. L.; Wells, J. A.; Shokat, K. M. K-Ras (G12C) inhibitors allosterically control GTP affinity and effector interactions. Nature 2013, 503, 548-551.

1. A salt of Compound A 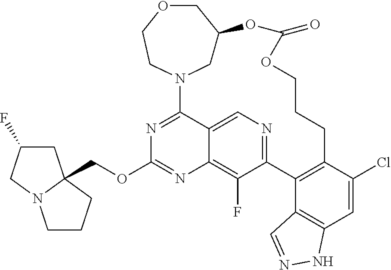 [SMILES: O=C1OCCCc2c(Cl)cc3[nH]ncc3c2-c2ncc3c(nc(OC[C@@]45CCCN4C[C@H](F)C5)nc3c2F)N2CCOC[C@H](C2)O1]
(Compound A), wherein the salt is a malonate salt.

2-5. (canceled)

6. The salt of claim 1, wherein the salt is an anhydrous malonate salt.

7. The salt of claim 1, characterized by one or more of the following data selected from the group consisting of:
(a) one or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα;
(b) one or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm;
(c) a differential scanning calorimetry (DSC) thermogram comprising an endotherm with an onset of about 170° C.±1° C.; and
(d) a thermogravimetric analysis (TGA) thermogram comprising a weight loss of about 13.4% when heated from about 170° C. to about 190° C.

8. The salt of claim 7, characterized by one or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

9. The salt of claim 8, characterized by two or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

10. The salt of claim 8, characterized by three or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

11. The salt of claim 8, characterized by four or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

12. The salt of claim 8, characterized by five or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

13. The salt of claim 8, characterized by seven or more X-ray powder diffraction (XRPD) peaks selected from: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

14. The salt of claim 8, characterized by X-ray powder diffraction (XRPD) peaks: 15.2, 16.7, 17.7, 18.6, 19.4, 20.6, 22.9, 23.3, 24.6, 28.0, and 29.5, ±0.2° 2θ using X-ray wavelength of 1.54 Å, wherein the source of radiation is CuKα.

15. The salt of claim 1, wherein the salt is a malonate salt of Compound A, characterized by an X-ray powder diffraction (XRPD) pattern comprising a peak at 16.7±0.2° 2θ using CuKα radiation.

16. The salt of claim 1, wherein the salt is a malonate salt of Compound A, characterized by an X-ray powder diffraction (XRPD) pattern comprising a peak at 16.7 and 22.9±0.2° 2θ using CuKα radiation.

17. The salt of claim 1, wherein the salt is a malonate salt of Compound A, characterized by an X-ray powder diffraction (XRPD) pattern comprising a peak at 16.7, 22.9, and 18.6±0.2° 2θ using CuKα radiation.

18. The salt of claim 17, further characterized by a peak at 19.4±0.2° 2θ using CuKα radiation.

19. The salt of claim 18, further characterized by a peak at 17.7±0.2° 2θ using CuKα radiation.

20. The salt of claim 19, further characterized by peaks at 24.6, 15.2, 23.3, and 28.0±0.2° 2θ using CuKα radiation.

21. The salt of claim 5, characterized by an X-ray powder diffraction (XRPD) pattern substantially as shown in FIG. 2.

22. The salt of claim 5, characterized by a differential scanning calorimetry (DSC) thermogram comprising an endotherm with an onset of about 170° C.±1° C.

23. The salt of claim 5, characterized by a differential scanning calorimetry (DSC) thermograph substantially as shown in FIG. 3.

24. The salt of claim 5, characterized by a thermogravimetric analysis (TGA) thermogram comprising a weight loss of about 13.4% when heated from about 170° C. to about 190° C.

25. The salt of claim 5, characterized by a thermogravimetric analysis (TGA) thermograph substantially as shown in FIG. 4.

26. The salt of claim 7, characterized by one or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm.

27. The salt of claim 26, characterized by two or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm.

28. The salt of claim 27, characterized by three or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm.

29. The salt of claim 28, characterized by four or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm.

30. The salt of claim 29, characterized by five or more ¹³C ssNMR spectrum peaks selected from: 78.7, 80.4, 92.7, 110.6, 118.4, 121.8, 131.0, 147.9, 154.5, 161.6, 163.2, 175.9, and 176.4, ±0.2 ppm.

31. The salt of claim 7, characterized by a ¹³C ssNMR spectrum comprising a peak at 176.4±0.2 ppm.

32. The salt of claim 7, characterized by a ¹³C ssNMR spectrum comprising a peak at 92.7 and 176.4±0.2 ppm.

33. The salt of claim 7, characterized by a ¹³C ssNMR spectrum comprising a peak at 92.7, 176.4, and 80.4±0.2 ppm.

34. The salt of claim 33, further characterized by a peak at 175.9±0.2 ppm.

35. The salt of claim 34, further characterized by a peak at 147.9±0.2 ppm.

36. The salt of claim 35, further characterized by peaks at 118.4, 121.8, and 154.5±0.2 ppm.

37. The salt of claim 36, further characterized by peaks at 78.7, 110.6, 131.0, 161.6 and 163.2±0.2 ppm.

38. The salt of claim 7, characterized by a ¹³C ssNMR spectrum substantially as shown in FIG. 5A.

39. The salt of claim 7, characterized by a dynamic vapor sorption (DVS) graph substantially as shown in FIG. 6.

40. The salt of claim 7, characterized by a ¹⁹F ssNMR spectrum substantially as shown in FIG. 78.

41-57. (canceled)

58. A pharmaceutical composition comprising the salt of claim and at least one pharmaceutically acceptable excipient.

59. A method of treating cancer in a human subject in need thereof comprising administering to the subject a therapeutically effective amount of the salt of claim or the pharmaceutical composition of claim 58.

60. The method of claim 59, wherein the cancer is non-small cell lung cancer, small bowel cancer, appendiceal cancer, colorectal cancer, cancer of unknown primary, endometrial cancer, mixed cancer types, pancreatic cancer, hepatobiliary cancer, small cell lung cancer, cervical cancer, germ cell cancer, ovarian cancer, gastrointestinal neuroendocrine cancer, bladder cancer, myelodysplastic/myeloproliferative neoplasms, head and neck cancer, esophagogastric cancer, soft tissue sarcoma, mesothelioma, thyroid cancer, leukemia, or melanoma.

61. The method according to claim 60, wherein the cancer is non-small cell lung cancer, colorectal cancer, pancreatic cancer, appendiceal cancer, endometrial cancer, esophageal cancer, cancer of unknown primary, ampullary cancer, gastric cancer, small bowel cancer, sinonasal cancer, bile duct cancer, or melanoma.

62. The method according to claim 61, wherein the cancer is non-small cell lung cancer.

63. The method according to claim 61, wherein the cancer is colorectal cancer.

64. The method according to claim 61, wherein the cancer is pancreatic cancer.

65. The method of claim 64, wherein the cancer is pancreatic ductal adenocarcinoma (PDAC).

66. (canceled)
